(19) United States
(12) Patent Application Publication
(10) Pub. No.: US 2024/0050589 A1
(43) Pub. Date: Feb. 15, 2024

(54) TREATMENT OF NEUROLOGICAL DISEASES

(71) Applicant: SHANGHAI GENEMAGIC BIOSCIENCES CO., LTD., Shanghai (CN)

(72) Inventors: Haibo ZHOU, Shanghai (CN); Xinde HU, Shanghai (CN); Jinlin SU, Shanghai (CN)

(21) Appl. No.: 18/259,090

(22) PCT Filed: Dec. 25, 2020
(86) PCT No.: PCT/CN2020/139705

§ 371 Date: Jun. 23, 2023

(51) Int. Cl. A61K 48/00; C12N 9/22; C12N 15/11; C12N 15/86; A61P 25/16

(52) U.S. Cl. CPC A61K 48/005; C12N 9/22; C12N 15/11; C12N 15/86; A61K 48/0075; A61P 25/16

(57) ABSTRACT

Provided are methods and composition for treating certain neurodegenerative diseases, such as RGC loss-related degenerative disease and Parkinson's Disease, using in vivo conversion of glial cells to neurons by PTB and optionally nPTB knock down via CRISPR/Cas delivered by AAV vectors.

### FIELD OF THE INVENTION

The present invention relates to the fields of regenerative medicine, neurology and biopharmaceuticals. In particular the invention relates to methods and compositions for differentiating non-neuronal cells to neuronal cells for treating neurodegenerative diseases and disorders.

### BACKGROUND ART

Neurodegenerative diseases are devastating diseases associated with the progressive loss of neurons in various parts of the nervous system. On the other hand, regenerative medicine has great promise for treating neurodegenerative diseases that lead to cell (e.g., neuron) loss. One approach employs cell replacement, while another utilizes cellular trans-differentiation.

Trans-differentiation takes advantage of the existing cellular plasticity of endogenous cells to generate new cell types. One challenge for this approach, however, is to identify efficient strategies to convert certain target cells to a desired cell type (e.g., neurons), not only in culture but more importantly in their in vivo native contexts, particularly at a desired location (e.g., a tissue or organ type).

Recent studies showed that down regulation of a single gene, polypyrimidine tract-binding protein 1 (Ptbp1), in the striatum could directly convert astrocytes into dopaminergic neurons in mice and alleviated the symptoms in a mouse model of Parkinson's disease (Zhou et al., 2020, Cell 181: 590-603). However, it is unknown whether this strategy could be applied in nonhuman primates and human. Many experimental therapies showing the effectiveness in rodent models may be inefficient or impractical in nonhuman primates and human; these strategies may fail for many reasons, including intrinsic biology differences and lack of scalability. Researches in nonhuman primates is a key step and can be of great value in addressing these issues before clinical trials.

It is therefore an object of the instant invention to provide for methods and compositions for differentiating non-neuronal cells to neuronal cells for treating neurodegenerative diseases and disorders in primates, i.e. in both human and non-human primates.

### SUMMARY OF THE INVENTION

In a first aspect the invention pertains to a methods of treating a neurological condition associated with degeneration of functional neurons in a region in the nervous system of a non-human primate or a human.

In one embodiment, the method comprises the administration to a non-neuronal cell in the region in the nervous system of a subject in need thereof, an effective amount of a composition comprising a nucleic acid molecule that targets a target sequence or its complement in a primate PTB gene, transcript or mRNA, to suppress the expression or activity of a primate PTB protein in the non-neuronal cell, thereby allowing the non-neural cell to reprogram into a functional neuron, wherein the target sequence is comprised in a sequence that is at least 95% identical to SEQ ID NO: 87, and wherein when the target sequence is targeted with a guide RNA (gRNA), the guide sequence of which has at least 10 nucleotides overlap with at least one of SEQ ID NO.'s: 1-86, that is co-expressed with CasRx in at least one of Cos7 and 293T cells, a relative expression of PTB mRNA of less than 0.5 is observed, as compared to a corresponding control cell expressing only CasRx.

| SEQ ID NO: 87: | |
|---|---|
| atggacggca ttgtcccaga tatagccgtt ggtacaaagc ggggatctga cgagcttttc | 60 |
| tctacttgtg tcactaacgg accgtttatc atgagcagca actcggcttc tgcagcaaac | 120 |
| ggaaatgaca gcaagaagtt caaaggtgac agccgaagtg caggcgtccc ctctagagtg | 180 |
| atccacatcc ggaagctccc catcgacgtc acggaggggg aagtcatctc cctggggctg | 240 |
| ccctttggga aggtcaccaa cctcctgatg ctgaagggga aaaaccaggc cttcatcgag | 300 |
| atgaacacgg aggaggctgc caacaccatg gtgaactact acacctcggt gacccctgtg | 360 |
| ctgcgcggcc agcccatcta catccagttc tccaaccaca aggagctgaa gaccgacagc | 420 |
| tctcccaacc aggcgcgggc ccaggcggcc ctgcaggcgg tgaactcggt ccagtcgggg | 480 |
| aacctggcct tggctgcctc ggcggcggcc gtggacgcag ggatggcgat ggccgggcag | 540 |
| agccccgtgc tcaggatcat cgtggagaac ctcttctacc ctgtgaccct ggatgtgctg | 600 |
| caccagattt tctccaagtt cggcacagtg ttgaagatca tcaccttcac caagaacaac | 660 |
| cagttccagg ccctgctgca gtatgcggac cccgtgagcg cccagcacgc caagctgtcg | 720 |
| ctggacgggc agaacatcta caacgcctgc tgcacgctgc gcatcgactt ttccaagctc | 780 |
| accagcctca acgtcaagta caacaatgac aagagccgtg actacacacg cccagacctg | 840 |
| ccttccgggg acagccagcc ctcgctggac cagaccatgg ccgcggcctt cggtgcacct | 900 |
| ggtataatct cagcctctcc gtatgcagga gctggtttcc ctcccacctt tgccattcct | 960 |
| caagctgcag gcctttccgt tccgaacgtc cacggcgccc tggcccccct ggccatcccc | 1020 |
| tcggcggcgg cggcagctgc ggcggcaggt cggatcgcca tcccgggcct ggcgggggca | 1080 |
| ggaaattctg tattgctggt cagcaacctc aacccagaga gagtcacacc ccaaagcctc | 1140 |
| tttattcttt tcggcgtcta cggtgacgtg cagcgcgtga agatcctgtt caataagaag | 1200 |
| gagaacgccc tagtgcagat ggcggacggc aaccaggccc agctggccat gagccacctg | 1260 |
| aacgggcaca agctgcacgg gaagcccatc cgcatcacgc tctcgaagca ccagaacgtg | 1320 |
| cagctgcccc gcgagggcca ggaggaccag ggcctgacca aggactacgg caactcaccc | 1380 |
| ctgcaccgct tcaagaagcc gggctccaag aacttccaga acatattccc gccctcggcc | 1440 |
| acgctgcacc tctccaacat cccgccctca gtctccgagg aggatctcaa ggtcctgttt | 1500 |
| tccagcaatg ggggcgtcgt caaaggattc aagttcttcc agaaggaccg caagatggca | 1560 |
| ctgatccaga tgggctccgt ggaggaggcg gtccaggccc tcattgacct gcacaaccac | 1620 |
| gacctcgggg agaaccacca cctgcgggtc tccttctcca agtccaccat ctag | 1674 |

In one embodiment, the method comprising the administration to a non-neuronal cell in the region in the nervous system of a subject in need thereof, an effective amount of a composition comprising a nucleic acid molecule that targets a target sequence or its complement in a primate PTB gene, transcript or mRNA, to suppress the expression or activity of PTB in the non-neuronal cell, thereby allowing the non-neural cell to reprogram into a functional neuron, wherein the target sequence is comprised in a sequence that is at least 95% identical to positions 951-1487 of SEQ ID NO: 87. In some embodiments of the method, when the target sequence is targeted with a guide RNA (gRNA), the guide sequence of which has at least 10 nucleotides overlap with at least one of SEQ ID NO.'s: 38-68, that is co-expressed with CasRx in at least one of Cos7 and 293T cells, a relative expression of PTB mRNA of less than 0.4 is observed, as compared to a corresponding control cell expressing only CasRx.

In one embodiment, the method is a method wherein the nucleic acid molecule is or encodes at least one of an antisense nucleic acid, an RNAi molecule or a guide RNA (gRNA). In some embodiments, In one embodiment, the method is a method wherein composition comprises: i) a Cas effector protein and at least one gRNA or ii) at least one expression vector encoding a Cas effector protein and encoding the at least one gRNA, wherein optionally, the Cas effector protein and the at least one gRNA or the at least one expression vector are comprised in a nanoparticle, preferably a liposome.

In one embodiment, the method is a method wherein: a) the Cas effector protein is an RNA-targeting Cas effector protein; and, b) the gRNA comprises a guide sequence that is complementary to a contiguous stretch of 17-60 nucleotides in a sequence that is at least 95% identical to SEQ ID NO: 87. In some embodiments, the method is a method wherein the RNA-targeting Cas effector protein selected from the group consisting of: Cas13d, CasRx, Cas13e, Cas13a, Cas13b, Cas13c, Cas13f and a functional domain thereof, of which CasRx is preferred. In some embodiments, the method is a method wherein the guide sequence in the gRNA comprises or consists of at least 10, 11, 12, 13, 14, 15, 16, 17 contiguous nucleotides, or of all nucleotides of at least one of SEQ ID NO.'s: 1-86, and wherein, preferably, when the gRNA is co-expressed with CasRx in at least one of Cos7 and 293T cells, a relative expression of PTB mRNA of less than 0.5, 0.45, 0.4, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx. In some embodiments, the method is a method wherein the guide sequence in the gRNA comprises or consists of at least one sequence selected from SEQ ID NO.'s: 56, 60, 47, 48, 43, 45, 40, 61, 50, 55, 41, 44, 42, 66, 49, 51 and 46.

In one embodiment, the method is a method wherein the composition comprises only a single type of gRNA or two, three, four five or six different gRNAs that target a primate PTB mRNA sequence or the at least one expression vector encodes only a single type of gRNA or two, three, four five or six different gRNAs that target a primate PTB mRNA sequence.

In one embodiment, the method is a method wherein the at least one expression vector comprises: i) a nucleotide sequence encoding the Cas effector protein that is operably linked to a promoter that causes expression of the Cas effector protein in a primate non-neuronal cell, wherein preferably the promoter is a glial cell-specific promoter or a Müller glia (MG) cell-specific promoter, wherein more preferably a glial cell-specific promoter selected from the GFAP promoter, the ALDH1L1 promoter, the EAAT1/GLAST promoter, the glutamine synthetase promoter, S100 beta promoter and the EAAT2/GLT-1 promoter, or an MG cell-specific promoter selected from the GFAP promoter, the ALDH1L1 promoter, Glast (also known as Slc1a3) promoter and the Rlbp1 promoter; and, ii) at least one nucleotide sequence encoding a gRNA that targets a primate PTB mRNA sequence, which nucleotide sequence is operably linked to a promoter that causes expression of the gRNA in the non-neuronal cell, such as a U6 promoter.

In one embodiment, the method is a method wherein the expression vector is comprised in a nanoparticle or wherein the expression vector is a gene therapy vector, preferably a viral gene therapy vector, more preferably a viral vector selected from the group consisting of an adeno-associated virus (AAV) vector, an adenovirus vector, a lentivirus vector, a retrovirus vector, a herpes virus, a SV40 vector, a poxvirus vector, and a combination thereof, of which AAV is most preferred.

In one embodiment, the method is a method wherein the composition is administered locally to at least one of: i) a non-neuronal cell in a mature retina; ii) a non-neuronal cell in the striatum, preferably putamen; iii) a non-neuronal cell in the substantia nigra, iv) a non-neuronal cell in an inner ear; v) a non-neuronal cell in the spinal cord; vi) a non-neuronal cell in the prefrontal cortex; vii) a non-neuronal cell in the motor cortex; and, viii) a non-neuronal cell in the ventral tegmental area (VTA). In some embodiments, the method is a method wherein the composition is administered to a non-neuronal cell in the striatum for generating a functional dopaminergic neuron, whereby, preferably the non-neuronal cell is a glial cell, wherein preferably the composition is administered to at least one of the putamen and substantia nigra. In a preferred embodiment, the glial cell is an astrocyte. In some embodiments, the method is a method wherein the neurological condition is a condition associated with degeneration of functional neurons is selected from the group consisting of: Parkinson's disease; Alzheimer's disease; Huntington's disease; Schizophrenia; depression; drug addiction; stroke; movement disorder such as chorea; spinal cord injury; choreoathetosis, and dyskinesias; bipolar disorder; Autism spectrum disorder (ASD); and dysfunction. In some embodiments, the method is a method wherein the composition further comprises i) one or more dopamine neuron-associated factors, or ii) at least one expression vector for expression of one or more dopamine neuron-associated factors in the non-neuronal cell. In a preferred embodiment, the one or more dopamine neuron-associated factors are selected from the group consisting of: Lmx1a, Lmx1b, FoxA2, Nurr1, Pitx3, Gata2, Gata3, FGF8, BMP, En1, En2, PET1, a Pax family protein, SHH, a Wnt family protein and a TGF-β family protein.

In one embodiment, the method is a method wherein the composition is administered to a glial cell or Müller glia (MG) cell in the mature retina for generating a functional retinal ganglion cell (RGC) neuron. In one embodiment, the method is a method wherein the composition is administered to a glial cell or Müller glia (MG) cell in the mature retina for generating a functional retinal photoreceptor. In some embodiments, the method is a method wherein the neurological condition is a condition associated with degeneration of functional neurons in the mature retina is selected from the group consisting of: glaucoma, age-related RGC loss, optic nerve injury, retinal ischemia and Leber's hereditary optic neuropathy. In one embodiment, the method is a method wherein the composition further comprises i) one or more factors selected from β-catenin, Oct4, Sox2, Klf4, Crx, Brn3a, Brn3b, Math5, Nr2e3 and Nrl, and/or ii) at least one expression vector for expression in a non-neuronal cell of one or more factors selected from β-catenin, Oct4, Sox2, Klf4, Crx, Brn3a, Brn3b, Math5, Nr2e3 and Nrl.

In one embodiment, the method is a method wherein the method further comprises administering an immunosuppressant, prior to, simultaneously with or after the administration of the cell-programming agent, wherein more preferably, the immunosuppressant is selected from the group consisting of: a corticosteroid, a calcineurin inhibitor, an mTOR inhibitor, an IMDH inhibitor, an immuno suppressive antibody, an interferon, a Janus kinase inhibitor and a biologic such as anakinra.

In a second aspect the invention pertains to a composition comprising a nucleic acid molecule that targets a target sequence or its complement in a primate PTB gene, transcript or mRNA, wherein the target sequence is as herein defined. In some embodiments, the composition comprises: i) a Cas effector protein and at least one gRNA or ii) at least one expression vector encoding a Cas effector protein and encoding the at least one gRNA, wherein optionally, the Cas effector protein and the at least one gRNA or the at least one expression vector are comprised in a nanoparticle, preferably a liposome. In preferred embodiments, the Cas effector protein and the at least one gRNA are as herein defined. In a preferred embodiment, the expression vector is an expression vector as herein defined.

In one embodiment, a composition for generating a functional dopaminergic neuron further comprises i) one or more dopamine neuron-associated factors, or ii) at least one expression vector for expression of one or more dopamine neuron-associated factors in the non-neuronal cell, wherein preferably, the one or more dopamine neuron-associated factors are selected from the group consisting of: Lmx1a, Lmx1b, FoxA2, Nurr1, Pitx3, Gata2, Gata3, FGF8, BMP, En1, En2, PET1, a Pax family protein, SHH, a Wnt family protein and a TGF-β family protein.

In one embodiment, a composition for generating a functional RGC neuron further comprises i) one or more factors selected from β-catenin, Oct4, Sox2, Klf4, Crx, Brn3a, Brn3b, Math5, Nr2e3 and Nrl, and/or ii) at least one expression vector for expression in a non-neuronal cell of one or more factors selected from β-catenin, Oct4, Sox2, Klf4, Crx, Brn3a, Brn3b, Math5, Nr2e3 and Nrl.

In one embodiment, the composition is formulated for injection, inhalation, parenteral administration, intravenous administration, subcutaneous administration, intramuscular administration, intradermal administration, topical administration, or oral administration.

In a third aspect, the invention pertains to an AAV vector, comprising: (a) a coding sequence of an RNA-targeting Cas effector protein; and (b) at least one nucleotide sequence encoding a gRNA as herein defined. In some embodiments, the AAV vector is a vector wherein the RNA-targeting Cas effector protein selected from the group consisting of: Cas13d, CasRx, Cas13e, Cas13a, Cas13b, Cas13c, Cas13f and a functional domain thereof, of which CasRx is preferred. In some embodiments, the AAV vector is a vector wherein: i) the nucleotide sequence encoding the Cas effector protein that is operably linked to a promoter that causes expression of the Cas effector protein in a primate non-neuronal cell, wherein preferably the promoter is a glial cell-specific promoter or a Müller glia (MG) cell-specific promoter, wherein more preferably a glial cell-specific promoter selected from the GFAP promoter, the ALDH1L1 promoter, the EAAT1/GLAST promoter, the glutamine synthetase promoter, S100 beta promoter and the EAAT2/GLT-1 promoter, or an MG cell-specific promoter selected from the GFAP promoter, the ALDH1L1 promoter, Glast (also known as Slc1a3) promoter and the Rlbp1 promoter; and, ii) the at least one nucleotide sequence encoding a gRNA is operably linked to a promoter that causes expression of the gRNA in the non-neuronal cell, such as a U6 promoter.

### DESCRIPTION OF DRAWINGS

FIG. 1 Screen of efficient gRNAs. (A) Downregulation of Ptbp1 mRNA with 86 gRNAs in human 293T cells, two days after transient transfection of plasmids encoding CasRx and gRNAs. Note that the gRNA 60 was used in the following experiments. (B) Targeting sites of 86 gRNAs in human Ptbp1 gene, as well the information of knockdown efficiency (dark, <0.1; lighter, >0.1). Key region indicates the targeting region with high knockdown efficiency. The key region corresponds to positions 951-1487 of SEQ ID NO: 87, which are position 1-536 of SEQ ID NO:88. All values are presented as mean±SEM.

FIG. 2 The gRNA 60 showed high knockdown efficiency in human 293T cells. All values are presented as mean±SEM.; unpaired t test; *p<0.05, **p<0.01, ***p<0.001.

FIG. 3 Knockdown of Ptbp1 expression in monkey and mouse cells using gRNA 60. (A, B) The gRNA 60 showed high knockdown efficiency in monkey Cos7 cells and Mouse N2a cells. All values are presented as mean±SEM.; unpaired t test; *p<0.05, **p<0.01, ***p<0.001.

FIG. 4 Expression levels in log 2 (fragments per kilo base per million mapped reads [FPKM]+1) values of all detected genes in RNA sequencing (RNA-seq) libraries of CasRx-Ptbp1 (y axis) compared to CasRx control (x axis), showing that Ptbp1 was specifically downregulated. Cos7 cells, n=3 independent replicates for both groups. The gRNA 60 was used.

FIG. 5 Conversion of astrocytes into dopamine neurons in vivo.
(A) Schematic illustration of the AAV vectors and injection strategy. Vector 1 (AAV-GFAP-mCherry) encodes mCherry driven by the Astrocyte-specific promoter GFAP and Vector 2 (AAV-GFAP-CasRx) encodes CasRx, and Vector 3 (AAV-GFAP-CasRx-Ptbp1) encodes CasRx and gRNAs. The striatum were either injected with AAV-GFAP-CasRx-Ptbp1 or control vector AAV-GFAP-CasRx together with AAV-GFAP-mCherry. Astrocyte-to-dopamine neuron conversion is evaluated around 2-3 weeks post-injection. ST, striatum.
(B) Representative Images showing that mCherry (Vector 1) and CasRx (Fused with Flag, vector 3) were specifically expressed in the mouse astrocytes. Note that GFAP is the astrocyte-specific marker. (C) and (D) Confocal images showing that converted mCherry⁺NeuN⁺ (white arrowheads) and mCherry⁺TH⁺ (white arrowheads) cells were observed in mice 2 weeks after vector 1+3 injection, but not in the striatum injected with control AAVs. Note that NeuN and TH are neuron- and dopamine neuron-specific markers, respectively. (E) Confocal images showing that converted mCherry⁺NeuN⁺ (white arrowheads) and mCherry⁺TH⁺ (white arrowheads) cells were observed in mice one month after vector 1+3 injection, but not in the control striatum. Note that DAT is the mature dopamine neuron-specific marker. (F) Representative images showing a mCherry⁺TH⁺ cell in the Macaca fascicularis putamen injected with vector 1 and 3 (yellow arrowhead). The gRNA 60 was used.

FIG. 6 Increase of astrocyte-to-dopamine neurons conversion efficiency by co-injection of dopamine neuron-associated transcription factors.

Confocal images showing that converted mCherry⁺NeuN⁺TH⁺(yellow arrowheads) cells were observed in mice 2-3 weeks after injection of AAV-GFAP-mCherry+AAV-GFAP-CasRx-Ptbp1, AAV-GFAP-mCherry+AAV-GFAP-CasRx-Ptbp1+AAV-GFAP-FoxA2, AAV-GFAP-mCherry+AAV-GFAP-CasRx-Ptbp1+AAV-GFAP-Lmx1a, AAV-GFAP-mCherry+AAV-GFAP-CasRx-Ptbp1+AAV-GFAP-FoxA2+AAV-GFAP-Lmx1a and AAV-GFAP-mCherry+AAV-GFAP-CasRx-Ptbp1+AAV-GFAP-FoxA2+AAV-GFAP-Lmx1a+AAV-GFAP-Nurr1, but not in the control striatum injected with AAV-GFAP-mCherry+AAV-GFAP-CasRx. Note that NeuN and TH are the neuron-specific and dopamine neuron-specific markers, respectively. White arrowhead indicates a mCherry⁺NeuN⁻TH⁻ Cell. The gRNA 60 was used.

FIG. 7

Increase of MG-to-RGC conversion efficiency by co-injecting β-catenin in middle-aged mice.
(A) Schematic showing MG-to-RGC conversion. Vector I (AAV-GFAP-GFP-Cre) expresses Cre recombinase and GFP, which were driven by the MG-specific promoter GFAP. Vector II (AAV-GFAP-CasRx-Ptbp1) expresses CasRx and gRNAs. To induce MG-to-RGC conversion, retinas (Ai9 mice, 4-5 months old) were injected with AAV-GFAPCasRx-Ptbp1 or control AAV-GFAP-CasRx together with AAV-GFAP-GFP-Cre. Induction of RGC conversion was examined 2-3 weeks post-injection. ONL, outer nuclear layer; OPL, outer plexiform layer; INL, inner nuclear layer; IPL, inner plexiform layer; GCL, ganglion cell layer.
(B-E) Representative images indicate colocalization of RBPMS+tdTomato+ cells in the GCL, and tdTomato+ optic nerve for AAV-GFAP-GFP-Cre+AAV-GFAP-CasRx, AAV-GFAP-GFP-Cre+AAV-GFAP-β-catenin, AAV-GFAP-GFP-Cre+AAV-GFAP-CasRx-Ptbp1 and AAV-GFAP-GFP-Cre+AAV-GFAP-CasRx-Ptbp1+AAV-GFAP-β-catenin groups. Note that AAV-GFAP-GFP-Cre+AAV-GFAP-CasRx and AAV-GFAP-GFP-Cre+AAV-GFAP-β-catenin are control groups. Yellow arrowhead showing the colocalization of tdTomato and RBPMS in the retinas injected with AAV-GFAP-GFP-Cre, AAV-GFAP-CasRx-Ptbp1 and AAV-GFAP-β-catenin. RBPMS is a specific marker for RGCs. The gRNA 60 was used

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

“Astrocyte” generally refer to characteristic star-shaped glial cells in the brain and spinal cord, that is characterized by one or more of: star shape; expression of markers like glial fibrillary acidic protein (GFAP), aldehyde dehydrogenase 1 family member LI (ALDH1L1), excitatory amino acid transporter 1/glutamate aspartate transporter (EAAT1/GLAST), glutamine synthetase, S100 beta, or excitatory amino acid transporter 1/glutamate transporter 1 (EAAT2/GLT-1); participation of blood-brain barrier together with endothelial cells; transmitter uptake and release; regulation of ionic concentration in extracellular space; reaction to neuronal injury and participation in nervous system repair; and metabolic support of surrounding neurons.

In certain embodiments, an astrocyte refers to a non-neuronal cell in a nervous system that expresses glial fibrillary acidic protein (GFAP), Aldehyde Dehydrogenase 1 Family Member L1 (ALDH1L1), or both.

In certain embodiments, an astrocyte refers to a non-neuronal cell in a nervous system that expresses a glial fibrillary acidic protein (GFAP) promoter-driven transgene (e.g., red fluorescent protein (RFP), Cre recombinase).

In certain embodiments, a Müller glia (MG) refers to a non-neuronal glial cell found in the retina and that expresses a MG-specific promoter-driven transgene (e.g., red fluorescent protein (RFP). MG-specific promoters include promoters from GFAP, Glast (also known as Slc1a3) and Rlbp1.

A “BRN2 transcription factor” or “Brain-2 transcription factor,” also called “POU domain, class 3, transcription factor 2” (“POU3F2”) or “Oct-7,” can refer to a class III POU domain transcription factor, having a DNA-binding POU domain that consists of an N-terminal POU-specific domain of about 75 amino acids and a C-terminal POU-homeo domain of about 60 amino acids, which are linked via a linker comprising a short a-helical fold, and which can be predominantly expressed in the central nervous system.

The term “cell-programming agent” generally refers to an agent that reprograms a differentiated non-neuronal cell to a neuronal cell, through inhibiting the expression and/or function of PTB and/or nPTB. In a specific embodiment, the cell-programming agent refers to a CRISPR/Cas effector protein (which may or may not include any variants, derivatives, functional equivalents or fragments thereof) with a guide RNA (gRNA) complementary to a PTB mRNA or to a nPTB mRNA, and can knock down the expression and/or activity of PTB or nPTB, to an extent sufficient to convert a non-neuronal cell to a neuronal cell, preferably in vivo at a local microenvironment where the converted neuron is expected to be functional. The cell-programming agent may also refer to a polynucleotide encoding such CRISPR/Cas effector protein as defined above and/or the guide RNA (gRNA). The polynucleotide may include an mRNA for the Cas effector as defined above. The polynucleotide may also include a DNA encoding the Cas effector as defined above and/or the gRNA complementary to PTB/nPTB mRNA. The DNA encoding the Cas effector as defined above and/or the gRNA may be part of a vector, including a viral vector (e.g., an AAV vector or a lentiviral vector, or any of the other viral vectors described herein below). In the case of AAV, any AAV with tropism for glial cell or non-neuronal cell in the CNS and/or PNS can be used, including AAV1, AAV2, AAV3, AAV4, AAV5, AAV6, AAV7, AAV8, AAV9, AAV10, AAV11, AAV12, AAV13, AAV14, AAV15, AAV16, etc. Also in the case of AAV, any Cas effector as defined above can be used, so long as the coding sequence for the Cas effector is smaller than the packaging capacity of AAV, such as 4.7 kb, 4.5 kb, 4.0 kb, 3.5 kb, 3.0 kb, 2.5 kb, 2.0 kb, 1.5 kb or less. Exemplary Cas effector that may be used with the invention includes Cas13a, Cas13b, Cas13c, Cas13d, CasRx, Cas13e, Cas13f, Cpf1, Cas9, and functional equivalent or fragment thereof. In certain narrowest sense, the term “cell-programming agent” may be used interchangeably with the Cas effector with the gRNA, or polynucleotide (e.g., DNA or vector) encoding the same.

The Cas effector protein that can be used with the invention described herein include CRISPR-Cas Class 2 systems utilizing a single large Cas protein to degrade target nucleic acids (e.g., mRNA). Suitable Class 2 Cas effectors may include Type II Cas effectors such as Cas9 (e.g., Streptococcus pyogenes SpCas9 and S. thermophilus Cas9). The suitable Cas effector may also be Class 2, type V Cas proteins, including Cas12a (formerly known as Cpf1, such as Francisella novicida Cpf1 and Prevotella Cpf1), C2c1 and C2c3, which lack HNH nuclease, but have RuvC nuclease activity. Particularly suitable Cas effector proteins may include Class 2, type VI Cas proteins, including Cas13 (also known as C2c2), Cas13a, Cas13b, Cas13c, Cas13d/CasRx, Cas13e and Cas13f, each is an RNA-guided RNase (i.e., these Cas proteins use their crRNA to recognize target RNA sequences, rather than target DNA sequences in Cas9 and Cas12a). Overall, the CRISPR/Cas13 systems can achieve higher RNA digestion efficiency compared to the traditional RNAi and CRISPRi technologies, while simultaneously exhibiting much less off-target cleavage compared to RNAi.

Thus in a specific embodiment, the cell-programming agent of the invention is or encodes a Cas effector protein that, together with its canonical gRNA, targets PTB or nPTB mRNA. In other embodiments, the Cas effector targets PTB or nPTB DNA.

The term “contacting” cells with a composition of the disclosure refers to placing the composition (e.g., compound, nucleic acid, viral vector etc.) in a location that will allow it to touch the cell in order to produce “contacted” cells. The contacting may be accomplished using any suitable method. For example, in one embodiment, contacting is by adding the compound to a culture of cells. Contacting may also be accomplished by injecting it or delivering the composition to a location within a body such that the composition “contacts” the cell type targeted.

The term “differentiation,” “differentiate,” or “converting,” or “inducing differentiation” are used interchangeably to refer to changing the default cell type (genotype and/or phenotype) to a non-default cell type (genotype and/or phenotype). Thus “inducing differentiation in an astrocyte cell” refers to inducing the cell to change its morphology from that of an astrocyte to that of a neuronal cell type (i.e., change in gene expression as determined by genetic analysis such as a microarray) and/or phenotype (i.e. change in expression of a protein).

As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which may be determined as genome copies per kilogram (GC/kg). Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of an mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in the art per se. Expression control sequences can be of such nature as to modulate the nucleotide sequence to which it is operably linked such that lower expression levels or higher expression levels are achieved.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “glial cell” can generally refer to a type of supportive cell in the central nervous system (e.g., brain and spinal cord) and the peripheral nervous system.

In some embodiments, glial cells do not conduct electrical impulses or exhibit action potential. In some embodiments, glial cells do not transmit information with each other, or with neurons via synaptic connection or electrical signals. In a nervous system or in an in vitro culture system, glial cells can surround neurons and provide support for and insulation between neurons. Non-limiting examples of glial cells include oligodendrocytes, astrocytes, Muller Glia, ependymal cells, Schwann cells, microglia, spiral ganglion glial cell and satellite cells.

A “guide sequence” is to be understood herein as a sequence that directs an RNA or DNA guided endonuclease to a specific site in an RNA or DNA molecule. In the context of a guide RNA (gRNA)-CAS complex, “guide sequence” is further to be understood herein as the section of the gRNA (or crRNA), which is required for targeting a gRNA-CAS complex to a specific site in the target RNA or DNA molecule. The “guide sequence” in a gRNA is complementary to that specific site in the target RNA or DNA molecule, which site is the “target sequence” (see also below).

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

A “microRNA” or “miRNA” refers to a non-coding nucleic acid (RNA) sequence that binds to at least partially complementary nucleic acid sequence (mRNAs) and negatively regulates the expression of the target mRNA at the post-transcriptional level. A microRNA is typically processed from a “precursor” miRNA having a double-stranded, hairpin loop structure to a “mature” form. Typically, a mature microRNA sequence is about 19-25 nucleotides in length.

“miR-9” is a short non-coding RNA gene involved in gene regulation and highly conserved from Drosophila and mouse to human. The mature ˜21nt miRNAs are processed from hairpin precursor sequences by the Dicer enzyme. miR-9 can be one of the most highly expressed microRNAs in developing and adult vertebrate brain. Key transcriptional regulators such as FoxGl, Hesl or Tlx, can be direct targets of miR-9, placing it at the core of the gene network controlling the neuronal progenitor state.

The term “neuron” or “neuronal cell” as used herein can have the ordinary meaning one skilled in the art would appreciate. In some embodiments, neuron can refer to an electrically excitable cell that can receive, process, and transmit information through electrical signals (e.g., membrane potential discharges) and chemical signals (e.g., synaptic transmission of neurotransmitters). As one skilled in the art would appreciate, the chemical signals (e.g., based on release and recognition of neurotransmitters) transduced between neurons can occur via specialized connections called synapses.

The term “mature neuron” can refer to a differentiated neuron. In some embodiments, a neuron is the to be a mature neuron if it expresses one or more markers of mature neurons, e.g. microtubule-associated protein 2 (MAP2) and Neuronal Nuclei (NeuN), neuron specific enolase (NSE), 160 kDa neurofilament medium, 200 kDa neurofilament heavy, postsynaptic density protein 95 (PDS-95), Synapsin I, Synaptophysin, glutamate decarboxylase 67 (GAD67), glutamate decarboxylase 67 (GAD65), parvalbumin, dopamine- and cAMP-regulated neuronal phosphoprotein 32 (DARPP32), vesicular glutamate transporter 1 (vGLUT1), vesicular glutamate transporter 2 (vGLUT2), acetylcholine, and tyrosine hydroxylase (TH), etc. . . . .

The term “functional neuron” can refer to a neuron that is able to send or receive information through chemical or electrical signals. In some embodiments, a functional neuron exhibits one or more functional properties of a mature neuron that exists in a normal nervous system, including, but not limited to: excitability (e.g., ability to exhibit action potential, e.g., a rapid rise and subsequent fall in voltage or membrane potential across a cellular membrane), forming synaptic connections with other neurons, pre-synaptic neurotransmitter release, and post-synaptic response (e.g., excitatory postsynaptic current or inhibitory postsynaptic current).

In some embodiments, a functional neuron is characterized in its expression of one or more

markers of functional neurons, including, but not limited to, synapsin, synaptophysin, glutamate decarboxylase 67 (GAD67), glutamate decarboxylase 67 (GAD65), parvalbumin, dopamine- and cAMP-regulated neuronal phosphoprotein 32 (DARPP32), vesicular glutamate transporter 1 (vGLUT1), vesicular glutamate transporter 2 (vGLUT2), acetylcholine, tyrosine hydroxylase (TH), dopamine, vesicular GABA transporter (VGAT), and gamma-aminobutyric acid (GABA).

The term “non-neuronal cell” can refer to any type of cell that is not a neuron. An exemplary non-neuronal cell is a cell that is of a cellular lineage other than a neuronal lineage (e.g. a hematopoietic lineage). In some embodiments, a non-neuronal cell is a cell of neuronal lineage but not a neuron, for example, a glial cell. In some embodiments, a non-neuronal cell is somatic cell that is not neuron, such as, but not limited to, glial cell, adult primary fibroblast, embryonic fibroblast, epithelial cell, melanocyte, keratinocyte, adipocyte, blood cell, bone marrow stromal cell, Langerhans cell, muscle cell, rectal cell, or chondrocyte. In some embodiments, a non-neuronal cell is from a non-neuronal cell line, such as, but not limited to, glioblastoma cell line, Hela cell line, NT2 cell line, ARPE19 cell line, or N2A cell line.

“Cell lineage” or “lineage” can denote the developmental history of a tissue or organ from the fertilized embryo.

“Neuronal lineage” can refer to the developmental history from a neural stem cell to a mature neuron, including the various stages along this process (as known as neurogenesis), such as, but not limited to, neural stem cells (neuroepithelial cells, radial glial cells), neural progenitors (e.g., intermediate neuronal precursors), neurons, astrocytes, oligodendrocytes, and microglia.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

The terms “nucleic acid” and “polynucleotide” as used interchangeably herein can refer to deoxyribonucleotides or ribonucleotides and polymers thereof in either single- or doublestranded form. The term can encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, locked nucleic acids (LNAs), and peptide-nucleic acids (PNAs).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer a exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant AAV proteins and/or nucleic acids.

“Oligodendrocyte” can refer to a type of glial call that can create myelin sheath that surrounds a neuronal axon to provide support and insulation to axons in the central nervous system. Oligodendrocyte can also be characterized in their expression of PDGF receptor alpha (PDGFR-α), SOXIO, neural/glial antigen 2 (NG2), Olig 1, 2, and 3, oligodendrocyte specific protein (OSP), Myelin basic protein (MBP), or myelin oligodendrocyte glycoprotein (MOG).

“Polypyrimidine tract binding protein” or “PTB” and its homolog neural PTB (nPTB) are both ubiquitous RNA-binding proteins. PTB can also be called polypyrimidine tract-binding protein 1, and in humans is encoded by the PTBP1 gene. PTBP1 gene belongs to the subfamily of ubiquitously expressed heterogeneous nuclear ribonucleoproteins (hnRNPs).

The hnRNPs are RNA-binding proteins and they complex with heterogeneous nuclear RNA (hnRNA). These proteins are associated with pre-mRNAs in the nucleus and appear to influence pre-mRNA processing and other aspects of mRNA metabolism and transport. PTB can have four repeats of quasi-RNA recognition motif (RRM) domains that bind RNAs. Consistent with its widespread expression, PTB can contribute to the repression of a large number of alternative splicing events. PTB can recognize short RNA motifs, such as UCUU and UCUCU, located within a pyrimidine-rich context and often associated with the polypyrimidine tract upstream of the 3′ splice site of both constitutive and alternative exons.

In some cases, binding site for PTB can also include exonic sequences and sequences in introns downstream of regulated exons.

In most alternative splicing systems regulated by PTB, repression can be achieved through the interaction of PTB with multiple PTB binding sites surrounding the alternative exon.

In some cases, repression can involve a single PTB binding site. Splicing repression by PTB can occur by a direct competition between PTB and U2AF65, which in turn can preclude the assembly of the U2 snRNP on the branch point. In some cases, splicing repression by PTB can involve PTB binding sites located on both sides of alternative exons, and can result from cooperative interactions between PTB molecules that would loop out the RNA, thereby making the splice sites inaccessible to the splicing machinery. Splicing repression by PTB can also involve multimerization of PTB from a high-affinity binding site that can create a repressive wave that covers the alternative exon and prevents its recognition.

PTB can be widely expressed in non-neuronal cells, while nPTB can be restricted to neurons. PTB and nPTB can undergo a programmed switch during neuronal differentiation. For example, during neuronal differentiation, PTB is gradually downregulated at the neuronal induction stage, coincidentally or consequentially, nPTB level is gradually up-regulated to a peak level. Later, when the neuronal differentiation enters into neuronal maturation stage, nPTB level experiences reduction after its initial rise and then returns to a relatively low level as compared to the its peak level during neuronal differentiation, when the cell develops into a mature neuron.

The sequences of PTB and nPTB are known (see, e.g., Romanelli et al. (2005) Gene 356:11-8; Robinson et al., PLoS One. (2008) 3(3):e1801. doi:10.1371/journal.pone.0001801; Makeyev et al., Mol. Cell (2007) 27(3):435-448); thus, one of skill in the art can design and construct gRNA molecules and the like to modulate, e.g., to decrease or inhibit, the expression of PTB/nPTB; to practice the methods of the invention.

The terms “protein,” “peptide,” and “polypeptide” are used interchangeably, and can refer to an amino acid polymer or a set of two or more interacting or bound amino acid polymers, without reference to a specific mode of action, size, 3-dimensional structure or origin.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, enhancers and any other sequences of nucleotides known to one of skill in the art to act directly or indirectly to regulate the amount of transcription from the promoter. A “constitutive” promoter is a promoter that is active in most tissues under most environmental and developmental conditions. An “inducible” promoter is a promoter that is environmentally or developmentally regulated, e.g. by the application of a chemical inducer or biological entity.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame. An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “reprogramming” or “trans-differentiation” can refer to the generation of a cell of a certain lineage (e.g., a neuronal cell) from a different type of cell (e.g., a fibroblast cell) without an intermediate process of de-differentiating the cell into a cell exhibiting pluripotent stem cell characteristics.

“Pluripotent” can refer to the ability of a cell to form all lineages of the body or soma (i.e., the embryo proper). Exemplary “pluripotent stem cells” can include embryonic stem cells and induced pluripotent stem cells.

The terms “sequence identity”, “homology” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The terms “subject” and “patient” as used interchangeably can refer to, except where indicated, mammals such as humans and non-human primates, as well as rabbits, rats, mice, goats, pigs, and other mammalian species. The term does not necessarily indicate that the subject has been diagnosed with a particular disease, but instead can refer to an individual under medical supervision.

A “target sequence” is to denote an order of nucleotides within a nucleic acid that is to be targeted (e.g. wherein an alteration is to be introduced or to be detected). In the context of a guide RNA (gRNA)-CAS complex, a “target sequence” is further to be understood herein as the section within the RNA or DNA molecule that is to be targeted by the gRNA-CAS complex by its complementarity to the “guide sequence” in the gRNA (see also above). Likewise an antisense oligonucleotide or miRNA is targeted by its complementarity to the “target sequence” within the RNA or DNA molecule that is to be targeted.

For example, the target sequence is an order of nucleotides comprised by a first strand of a DNA duplex.

For example, mammalian species that benefit from the disclosed methods and composition include, but are not limited to, primates, such as apes, chimpanzees, orangutans and humans.

A “vector” is a nucleic acid that can be capable of transporting another nucleic acid into a cell. A vector can be capable of directing expression of a protein or proteins encoded by one or more genes, or a microRNA encoded by a polynucleotide, carried by the vector when it is present in the appropriate environment.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

A “viral vector” is a viral-derived nucleic acid that can be capable of transporting another nucleic acid into a cell. A viral vector can be capable of directing expression of a protein or proteins encoded by one or more genes, or a microRNA encoded by a polynucleotide, carried by the vector when it is present in the appropriate environment. Examples of viral vectors include, but are not limited to, retroviral, adenoviral, lentiviral and adeno-associated viral vectors.

### DETAILED DESCRIPTION OF THE INVENTION

### General Methods and Compositions

In one aspect, the invention relates to a method of treating a neurological condition associated with degeneration of functional neurons in a region in the nervous system of a non-human primate or a human. The method preferably comprising the administration to a subject in need thereof an effective amount of a composition comprising a cell-programming agent. In one embodiment, the composition comprising a cell-programming agent at least comprises a nucleic acid molecule that targets a target sequence or its complement in a primate PTB gene, transcript or mRNA, to suppress the expression or activity of PTB in the non-neuronal cell, thereby allowing the non-neural cell to reprogram into a functional neuron. The nucleic acid molecule that targets a target sequence or its complement in a primate PTB gene, transcript or mRNA, can thus be or can encode at least one of an antisense nucleic acid (e.g. for inducing exon-skipping), an miRNA or guide RNA (gRNA, for guiding the nuclease activity of a CRISPR/Cas effector protein).

In one embodiment, the nucleic acid molecule in the composition comprising the cell-programming agent is a nucleic acid molecule that targets a target sequence in a primate PTB gene, transcript or mRNA or in case of a double-stranded PTB DNA or RNA sequence, the nucleic acid molecule can target either one of the two complementary strands. Targeting of the target sequence (or its complement) in a primate PTB gene, transcript or mRNA by the nucleic acid molecule is understood to mean that at least a part of the nucleic acid molecule is substantially complementary to the target sequence in the primate PTB nucleic acid, such that can base pair with the primate PTB nucleic acid, preferably under physiological conditions, so as to exerts its biological effect, i.e. to suppress the expression or activity of PTB in the non-neuronal cell.

The inventors have identified target sequences within a primate or human PTB gene, transcript or mRNA effeciciency target sequence, the targeting of which by the nucleic acid molecules of the invention ensures high effciciency suppression of the expression or activity of PTB in the non-neuronal cell, as will be further detailed herein below.

In one embodiment, the composition comprising a cell-programming agent comprises: i) a CRISPR/Cas effector protein and a gRNA complementary to a Polypyrimidine Tract Binding protein (PTB) mRNA or ii) at least one expression vector encoding a CRISPR/Cas effector protein and a guide RNA (gRNA) complementary to a PTB mRNA.

Specifically, one aspect of the invention provides methods of reprogramming a non-neuronal cell to a mature neuron. An exemplary method comprises: providing a non-neuronal cell, and contacting the non-neuronal cell with a composition comprising a cell-programming agent (such as CRISPR/Cas effector with guide RNA (gRNA) or polynucleotide encoding the same) that suppresses expression and/or activity of PTB and/or nPTB in the non-neuronal cell, thereby reprogramming the non-neuronal cell to a mature neuron. The methods and compositions not only convert cells in vitro but also directly in vivo in nervous system (such as in striatum, retina, inner ear and spinal cord).

In some embodiment, the invention pertains to a method of treating a neurological condition associated with degeneration of functional neurons in a region in the nervous system of a non-human primate or a human, the method comprising the administration to a non-neuronal cell in the region in the nervous system of a subject in need thereof, an effective amount of a composition comprising i) an RNA-targeting Cas effector protein and a guide RNA (gRNA) that targets a primate PTB mRNA sequence or ii) at least one expression vector encoding an RNA-targeting Cas effector protein and encoding a gRNA that targets a primate PTB mRNA sequence, to suppress the expression or activity of PTB in the non-neuronal cell, thereby allowing the non-neural cell to reprogram into a functional neuron.

According to some embodiments of the disclosure, a single cell-programming agent (e.g., Cas/gRNA) that suppresses the expression and/or activity of PTB/nPTB in a non-human primate or human non-neuronal cell (e.g., MG cell in mature retina or astrocyte in striatum) can directly convert the non-neuronal cell into a mature neuron (e.g., a retinal ganglion cell (RGC) neuron, a retinal photoreceptor, or dopamine neuron, respectively). In some embodiments, the direct conversion of a non-neuronal cell into a neuron by a single cell-programming agent (e.g., Cas/gRNA) can mean that the conversion of the non-neuronal cell into the neuron requires no other intervention than contacting with the single cell-programming agent.

In another embodiment, the disclosure provides a method of reprogramming an astrocyte to a mature neuron. An exemplary method comprises: providing the astrocyte to be reprogrammed; and contacting the astrocyte with a composition comprising a cell-programming agent (e.g., Cas with PTB/nPTB-targeting gRNA or polynucleotide encoding the same) that suppresses the expression or activity of PTB in the astrocyte for at least 1 day, thereby reprogramming the astrocyte to a mature neuron such as a dopamine neuron. In some embodiments, a single cell-programming agent (e.g., Cas with PTB-targeting gRNA) that suppresses the expression or activity of PTB in an astrocyte can directly convert the astrocyte into a neuron such as a dopamine neuron. In some embodiments, the astrocyte is in striatum.

In another embodiment, the invention provides a method of reprogramming an MG cell (e.g., one in mature retina) to an RGC neuron. An exemplary method comprises: providing the MG cell to be reprogrammed; and contacting the MG cell with a composition comprising a cell-programming agent (e.g., Cas with PTB/nPTB-targeting gRNA or polynucleotide encoding the same) that suppresses the expression or activity of PTB and/or nPTB in the MG cell for at least 1 day, thereby reprogramming the MG cell to an RGC neuron. In some embodiments, a single cell-programming agent (e.g., Cas with PTB-targeting gRNA) that suppresses the expression or activity of PTB in an MG cell can directly convert the MG cell into an RGC neuron. In some embodiments, the MG cell is in mature retina.

According to the disclosure, in some cases, PTB reduction can induce a number of key neuronal differentiation factors. For example, without wishing to be bound to a certain theory, PTB and nPTB can be involved in two separate but intertwined loops, separately, that can be important in neuronal differentiation. PTB can suppress a neuronal induction loop in which the microRNA miR-124 can inhibit the transcriptional repressor RE1-Silencing Transcription factor (REST), which in turn can block the induction of miR-124 and many neuronal-specific genes (loop I). During a normal neuronal differentiation process, PTB can be gradually downregulated, and the PTB down-regulation can thus induce the expression of nPTB, which is part of a second loop for neuronal maturation that includes the transcription activator Brn2 and miR-9 (loop II). In loop II, nPTB can inhibit Brn2 and consequentially can inhibit miR-9, and miR-9 in turn can inhibit nPTB.

According to some embodiments, the expression level of miR-9 or Brn2 in a non-neuronal cell can affect the conversion of the non-neuronal cell into a mature neuron by a cell-programming agent that suppresses the expression or activity of PTB in the non-neuronal cell. For example, a human adult fibroblast cell can have a low expression level of miR-9 and Brn2. In some embodiments, a single agent that suppresses the expression or activity of PTB in a human adult fibroblast cell can induce the human adult fibroblast cell to differentiate into a neuron-like cell, e.g., expression of Tuj1 protein, but not into a mature neuron, e.g., expression of NeuN protein or other markers of a mature neuron.

Without wishing to be bound by a particular theory, the subject method and composition in some embodiments are particularly effective in creating a reinforcing feedback loop in molecular changes that direct the conversion of a non-neuronal cell into a neuron. Without wishing to be bound by a particular theory, when PTB expression or activity is initially downregulated by an exogenous anti-PTB agent, REST level can be downregulated, which can in turn lead to upregulation of miR-124 level.

Without wishing to be bound by a particular theory, in some cases, because miR-124 can target and inhibit the expression of PTB, the upregulated miR-124 can thus reinforce the inhibition of PTB in the cell; such a positive reinforcing effect can be long-lasting, even though in some cases, the anti-PTB agent, e.g., an antisense oligonucleotide against PTB, may be present and active merely temporarily in the cell.

According to some embodiments of the disclosure, a single cell-programming agent (e.g., Cas with PTB/nPTB-targeting gRNA or polynucleotide encoding the same) that suppresses the expression or activity of PTB/nPTB in a human non-neuronal cell can directly convert the non-neuronal cell into a mature neuron, optionally when the human non-neuronal cell expresses miR-9 or Brn2 at a level that is higher than that expressed in a human adult fibroblast.

An exemplary human non-neuronal cell that can be used in the method provided herein expresses miR-9 or Brn2 at a level that is at least two times higher than that expressed in a human adult fibroblast. In some embodiments, the human non-neuronal cell expresses miR-9 or Brn2 at a level that is at least about 1.2, 1.5, 2, 5, 10, 15, 20 or 50 times higher than that expressed in a human adult fibroblast.

In some embodiments, a single cell-programming agent that suppresses the expression or activity of PTB/nPTB (e.g., Cas with PTB/nPTB-targeting gRNA) in a human non-neuronal cell can directly convert the non-neuronal cell into a mature neuron, when the human non-neuronal cell expresses both miR-9 and Brn2 at a level that is higher than that expressed in a human adult fibroblast.

An exemplary human non-neuronal cell that can be used in the method as provided herein express both miR-9 and Brn2 at a level that is at least two times higher than that expressed in a human adult fibroblast. In some embodiments, the human non-neuronal cell expresses both miR-9 and Brn2 at a level that is at least about 1.2, 1.5, 2, 5, 10, 15, 20 or 50 times higher than that expressed in a human adult fibroblast.

In some embodiments, a single cell-programming agent (e.g., Cas with PTB/nPTB-targeting gRNA) that suppresses the expression or activity of PTB/nPTB in a human non-neuronal cell can directly convert the non-neuronal cell into a mature neuron, when the human non-neuronal cell expresses endogenous miR-9 or endogenous Brn2 at a level that is higher than that expressed in a human adult fibroblast. In some embodiments, no exogenous miR-9 is introduced into the human non-neuronal cell. In some embodiments, no exogenous Brn2 is introduced into the human non-neuronal cell.

In some embodiments, the expression level of miR-9 or Brn 2 in a non-neuronal cell can be assessed by any technique one skilled in the art would appreciate. For example, the expression level of miR-9 in a cell can be measured by reverse transcription (RT)-polymerase chain reaction (PCR), miRNA array, RNA sequencing (RNA-seq), and multiplex miRNA assays. Expression level of miR-9 can also be assayed by in situ methods like in situ hybridization. Expression level of Brn2 as a protein can be assayed by conventional techniques, like Western blot, enzyme-linked immunosorbent assay (ELISA), and immunostaining, or by other techniques, such as, but not limited to, protein microarray, and spectrometry methods (e.g., high performance liquid chromatography (HPLC) and liquid chromatography-mass spectrometry (LC/MS)). In some embodiments, information on the expression level of miR-9 in a cell or a certain type of tissue/cells can be obtained by referring to publicly available databases for microRNAs, such as, but not limited to, Human MiRNA Expression Database (HMED), miRGator 3.0, miRmine, and PhenomiR. In some embodiments, information on the expression level of miR-9 in a cell or a certain type of tissue/cells can be obtained by referring to publicly available databases for protein expression, including, but not limited to, The Human Protein Atlas, GeMDBJ Proteomics, Human Proteinpedia, and Kahn Dynamic Proteomics Database.

According to certain embodiments, an exemplary method comprises providing a human non-neuronal cell to be reprogrammed; and contacting the human non-neuronal cell with a composition comprising a single cell-programming agent (e.g., Cas with PTB/nPTB-targeting gRNA) that yields a decrease in expression or activity of PTB in the human non-neuronal cell, and a decrease of expression or activity of nPTB after the expression or activity of PTB is decreased. In some embodiments, the cell-programming agent can lead to a sequential event as to the expression or activity levels of PTB and nPTB in a certain type of non-neuronal cell, e.g., human non-neuronal cell, e.g., human glial cell. In some embodiments, the direct effect of contacting with the cell-programming agent (e.g., Cas with PTB-targeting gRNA) is a decrease of expression or activity of PTB in the non-neuronal cell. In some embodiments, in the non-neuronal cell, the decrease of expression or activity of PTB in the non-neuronal cell accompanies an initial increase of nPTB expression level in the non-neuronal cell. In some embodiments, an initial nPTB expression level increases to a high nPTB expression level as expression or activity of PTB is suppressed. In some embodiments, following the initial increase, nPTB expression decreases from the high nPTB expression level to a low nPTB expression level. In some embodiments, the low nPTB expression level is still higher than the initial nPTB expression level after expression or activity of PTB is suppressed. In some embodiments, the nPTB expression level decreases after the initial increase spontaneously without external intervention other than the cell-programming agent that suppresses the expression or activity of PTB. Without being bound to a certain theory, the subsequent decrease of nPTB expression level in the non-neuronal cell after PTB expression or activity is decreased by the cell-programming agent can be correlated with the direct conversion of the non-neuronal cell to a mature neuron by the cell-programming agent. According to some embodiments, a single cell-programming agent (e.g., Cas with PTB-targeting gRNA) that suppresses the expression or activity of PTB does not induce the sequential event as described above in a human adult fibroblast cell, e.g., nPTB can experience the initial rise in expression level, but no subsequent decrease to a certain low level.

In some embodiments, in a human astrocyte, a single cell-programming agent (e.g., Cas with PTB-targeting gRNA) that suppresses the expression or activity of PTB in the human astrocyte leads to immediate decrease in expression or activity of PTB, an initial increase in expression level of nPTB, and a subsequent decrease in expression level of nPTB. In some embodiments, a single cell-programming agent (e.g., Cas with PTB-targeting gRNA) that suppresses the expression or activity of PTB directly converts a human astrocyte to a mature neuron. In some embodiments, the expression level of miR-9 or Brn2 in the non-neuronal cell can be correlated with whether or not nPTB expression level in the non-neuronal decreases after the initial increases following PTB expression or activity is suppressed by a cell-programming agent. For instance, in human astrocyte, where miR-9 or Brn2 is expressed at a higher level than a human adult fibroblast, nPTB expression level in the non-neuronal decreases after the initial increases following PTB expression or activity is suppressed by a cell-programming agent, while in human adult fibroblast, as described above, in some cases, the subsequent decrease in nPTB expression level may not happen.

According to some embodiments, an exemplary non-neuronal cell that can be reprogrammed into a mature neuron in the method provided herein can include a glial cell, such as, but not limited, astrocyte, oligodendrocyte, ependymal cell, Microglia, Muller glia, spiral ganglion glial cell, Schwan cell, NG2 cells, and satellite cell. In some embodiments, a glial cell can be a primate glial cell, for instance, human glial cell or a non-human primate glial cell. Preferably the glial cell is a primate astrocyte, for instance, human astrocyte or a non-human primate astrocyte.

In some embodiments, a glial cell that can be used in the method as provided herein is a glial cell isolated from a brain. In some embodiments, a glial cell is a glial cell in a cell culture, for instance, divided from a parental glial cell. In some embodiments, a glial cell as provided herein is a glial cell differentiated from a different type of cell under external induction, for instance, differentiated in vitro from a neuronal stem cell in a culture medium containing differentiation factors, or differentiated from an induced pluripotent stem cell. In some other embodiments, a glial cell is a glial cell in a nervous system, for example, a MG cell in the mature retina, or an astrocyte residing in a brain region, such as in the striatum.

In some embodiments, an astrocyte that can be used in the method as provided herein is a glial cell that is of a star-shape in brain or spinal cord. In some embodiments, an astrocyte expresses one or more of well-recognized astrocyte markers, including, but not limited to, glial fibrillary acidic protein (GFAP) and aldehyde dehydrogenase 1 family member LI (ALDH1L1), excitatory amino acid transporter 1/glutamate aspartate transporter (EAAT1/GLAST), glutamine synthetase, S100 beta, or excitatory amino acid transporter 1/glutamate transporter 1 (EAAT2/GLT-1). In some embodiments, an astrocyte expresses glial fibrillary acidic protein (GFAP), Aldehyde Dehydrogenase 1 Family Member LI (ALDH1L1), or both. In certain embodiments, an astrocyte is a non-neuronal cell in a nervous system that expresses a glial fibrillary acidic protein (GFAP) promoter-driven transgene (e.g., red fluorescent protein (RFP), Cre recombinase). In some embodiments, an astrocyte as described herein is not immunopositive for neuronal markers, e.g., Tuj1, NSE, NeuN, GAD67, VGluTI, or TH. In some embodiments, an astrocyte as described herein is not immunopositive for oligodendrocyte markers, e.g., Oligodendrocyte Transcription Factor 2, OLIG2. In some embodiments, an astrocyte as described herein is not immunopositive for microglia markers, e.g., transmembrane protein 119 (TMEM119), CD45, ionized calcium binding adapter molecule 1 (Ibal), CD68, CD40, F4/80, or CD11 Antigen-Like Family Member B (CDllb). In some embodiments, an astrocyte as described herein is not immunopositive for NG2 cell markers (e.g., Neural/glial antigen 2, NG2). In some embodiments, an astrocyte as described herein is not immunopositive for neural progenitor markers, e.g., Nestin, CXCR4, Musashi, Notch-1, SRY-Box 1 (SOX1), SRY-Box 2 (SOX2), stage-specific embryonic antigen 1 (SSEA-1, also called CD15), or Vimentin. In some embodiments, an astrocyte as described herein is not immunopositive for pluripotency markers, e.g., NANOG, octamer-binding transcription factor 4 (Oct-4), SOX2, Kruppel Like Factor 4 (KLF4), SSEA-1, or stage-specific embryonic antigen 4 (SSEA-4). In some embodiments, an astrocyte as described herein is not immunopositive for fibroblast markers (e.g. Fibronectin).

Astrocytes can include different types or classifications. The methods of the invention are applicable to different types of astrocytes. Non-limiting example of different types of astrocytes include type 1 astrocyte, which can be Ran2⁺, GFAP+, fibroblast growth factor receptor 3 positive (FGFR3⁺), and A2B5. Type 1 astrocytes can arise from the tripotential glial restricted precursor cells (GRP). Type 1 astrocytes may not arise from the bipotential 02A/0PC (oligodendrocyte, type 2 astrocyte precursor) cells. Another non limiting example includes type 2 astrocyte, which can be A2B5⁺, GFAP⁺, FGFR3⁻, and Ran2. Type 2 astrocytes can develop in vitro from either tripotential GRP or from bipotential 02A cells or in vivo when these progenitor cells are transplanted into lesion sites. Astrocytes that can be used in the method provided herein can be further classified based their anatomic phenotypes, for instance, protoplasmic astrocytes that can be found in grey matter and have many branching processes whose end-feet envelop synapses; fibrous astrocyte that can be found in white matter and can have long thin unbranched processes whose end-feet envelop nodes of Ranvier. Astrocytes that can be used in the methods provided herein can also include GluT type and GluR type. GluT type astrocytes can express glutamate transporters (EAAT1/SLC1A3 and EAAT2/SLC1A2) and respond to synaptic release of glutamate by transporter currents, while GluR type astrocytes can express glutamate receptors (mostly mGluR and AMPA type) and respond to synaptic release of glutamate by channel mediated currents and IP3-dependent Ca²⁺ transients.

### A Cell-Programming Agent, Components Thereof and Compositions Comprising the Same

As provided herein, a composition comprising a cell-programming agent comprises nucleic acid molecule that targets a target sequence or its complement in a primate PTB gene, transcript or mRNA, to suppress the expression or activity of PTB in the non-neuronal cell, thereby allowing the non-neural cell to reprogram into a functional neuron. In one embodiment, the nucleic acid molecule is or encodes at least one antisense nucleic acid, RNAi or guide RNA (gRNA) that that targets a target sequence or its complement in a primate PTB gene, transcript or mRNA.

In certain embodiments, the nucleic acid molecule (e.g. gRNA) targets a target sequence in a primate PTB gene sequence, such as a non-human primate PTB gene sequence or preferably a human PTB gene sequence. Examples of primate PTB gene sequences to be targeted include e.g. the human PTBP1 gene sequence (e.g., GenBank ID 5725).

The sequences of primate and human PTB and nPTB are known (see e.g., Romanelli et al. (2005) Gene, August 15:356:11-8; Robinson et al., PLoS One. 2008 Mar. 12; 3(3):e1801. doi: 10.1371/journal.pone.0001801; Makeyev et al., Mol. Cell (2007) Aug. 3; 27(3):435-48); thus, one of skill in the art can design and construct antisense, miRNA, siRNA guide RNA molecules and the like to modulate, e.g., to decrease or inhibit, the expression of a primate PTB and/or nPTB; to practice the methods of this invention.

In some embodiments, the nucleic acid molecule (e.g. gRNA) targets a target sequence in the primate PTB gene, transcript or mRNA sequence that is conserved between humans and non-human primates.

In certain embodiments, the nucleic acid molecule (e.g. gRNA) targets a target sequence in a primate PTB mRNA sequence, such as a non-human primate PTB mRNA sequence or, preferably, a human PTB mRNA sequence. In some embodiment, the nucleic acid molecule (e.g. gRNA) targets a target sequence in the protein coding sequence in the primate mRNA sequence.

In certain embodiments, the target sequence in the PTB gene, transcript or mRNA is comprised in a PTB sequence that is at least 95, 96, 97, 98, 99 or 100% identical to SEQ ID NO: 87. A preferred target sequence is a target sequence, the targeting of which causes an efficient suppress the expression or activity of a primate PTB protein in a non-neuronal cell. In one embodiment therefore, the target sequence in the PTB gene, transcript or mRNA is comprised in a PTB sequence that is at least 95, 96, 97, 98, 99 or 100% identical to SEQ ID NO: 87, wherein when the target sequence is targeted with a guide RNA (gRNA), the guide sequence of which has at least 10 nucleotides overlap with at least one of SEQ ID NO.'s: 1-86, that is co-expressed with CasRx in at least one of Cos7 and 293T cells, a relative expression of PTB mRNA of less than 0.5, 0.5, 0.45, 0.4, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx. More preferably, the target sequence in the PTB gene, transcript or mRNA is comprised in a PTB sequence that is at least 95, 96, 97, 98, 99 or 100% identical to SEQ ID NO: 87, wherein when the target sequence is targeted with a guide RNA (gRNA), the guide sequence of which comprises or consists of at least one of SEQ ID NO.'s: 1-86, that is co-expressed with CasRx in at least one of Cos7 and 293T cells, a relative expression of PTB mRNA of less than 0.5, 0.5, 0.45, 0.4, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx.

In one embodiment, the target sequence in the PTB gene, transcript or mRNA comprises or consists of a contiguous sequence of at least 10, 11, 12, 13, 14, 15, 16, 17, 18, 19, 20, 21, 22, 23, 24 or 25 nucleotides and/or not more than 60, 55, 50, 45, 40, 35, 34, 33, 32, 31, 30, 29, 28, 27 or 26 nucleotides in a PTB sequence that is at least 95, 96, 97, 98, 99 or 100% identical to SEQ ID NO: 87.

In one embodiment, the target sequence in the PTB gene, transcript or mRNA comprises or consists of a contiguous sequence of at least 10, 11, 12, 13, 14, 15, 16, 17, 18, 19, 20, 21, 22, 23, 24 or 25 nucleotides and/or not more than 60, 55, 50, 45, 40, 35, 34, 33, 32, 31, 30, 29, 28, 27 or 26 nucleotides in a PTB sequence that is at least 95, 96, 97, 98, 99 or 100% identical to positions 951-1487 of SEQ ID NO: 87, and wherein preferably, when the target sequence is targeted with a guide RNA (gRNA), the guide sequence of which comprises or consists of at least one of SEQ ID NO.'s: 38-68, that is co-expressed with CasRx in at least one of Cos7 and 293T cells, a relative expression of PTB mRNA of less than 0.50, 0.45, 0.40, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx.

In one embodiment, the target sequence in the PTB gene, transcript or mRNA comprises or consists of a contiguous sequence of at least 10, 11, 12, 13, 14, 15, 16, 17, 18, 19, 20, 21, 22, 23, 24 or 25 nucleotides and/or not more than 60, 55, 50, 45, 40, 35, 34, 33, 32, 31, 30, 29, 28, 27 or 26 nucleotides in a PTB sequence that is at least 95, 96, 97, 98, 99 or 100% identical to at least one of: positions 1000-1487 of SEQ ID NO: 87; positions 1050-1487 of SEQ ID NO: 87; positions 1100-1487 of SEQ ID NO: 87; positions 1150-1487 of SEQ ID NO: 87; positions 1200-1487 of SEQ ID NO: 87; positions 1250-1487 of SEQ ID NO: 87; positions 1300-1487 of SEQ ID NO: 87; positions 1350-1487 of SEQ ID NO: 87; positions 1400-1487 of SEQ ID NO: 87; positions 951-1400 of SEQ ID NO: 87; positions 951-1350 of SEQ ID NO: 87; positions 951-1300 of SEQ ID NO: 87; positions 951-1250 of SEQ ID NO: 87; positions 951-1200 of SEQ ID NO: 87; positions 951-1150 of SEQ ID NO: 87; positions 951-1100 of SEQ ID NO: 87; positions 951-1050 of SEQ ID NO: 87; and positions 951-1000 of SEQ ID NO: 87; and wherein preferably, when the target sequence is targeted with a guide RNA (gRNA), the guide sequence of which comprises or consists of at least one of SEQ ID NO.'s: 38-68, that is co-expressed with CasRx in at least one of Cos7 and 293T cells, a relative expression of PTB mRNA of less than 0.50, 0.45, 0.40, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx.

Nucleic acid molecules or nucleic acid sequences used for targeting a primate PTB target sequence in accordance with this invention can be an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. Nucleic acid molecules use to practice this invention include “nucleic acids” or “nucleic acid sequences” including oligonucleotide, nucleotide, polynucleotide, or any fragment of any of these; and include DNA or RNA (e.g., mRNA, rRNA, tRNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs). Nucleic acid molecules use to practice this invention include nucleic acids, i.e., oligonucleotides, containing known analogues of natural nucleotides. Nucleic acid molecules use to practice this invention include nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. Nucleic acid molecules use to practice this invention include “oligonucleotides” including a single stranded polydeoxynucleotide or two complementary polydeoxynucleotide strands that may be chemically synthesized. Nucleic acid molecules use to practice this invention include synthetic oligonucleotides having no 5′ phosphate, and thus will not ligate to another oligonucleotide without adding a phosphate with an ATP in the presence of a kinase. A synthetic oligonucleotide can ligate to a fragment that has not been dephosphorylated.

In one embodiment, a nucleic acid molecule used for targeting a primate PTB target sequence in accordance with this invention is an antisense inhibitory nucleic acid molecules. In some embodiments, the invention thus provides antisense or otherwise inhibitory nucleic acid molecules capable of decreasing or inhibiting expression of a PTB and/or a nPTB gene or protein. In one embodiment, an antisense inhibitory nucleic acid molecule is capable of decreasing or inhibiting expression of a PTB and/or a nPTB gene or protein by inducing exon-skipping of a PTB pre-MRNA. In some embodiments, methods of the invention comprise use of molecules that can generate a PTB and a nPTB knockdown, or abrogation or significant decrease in PTB and nPTB expression. In some embodiments, methods of the invention comprise use of these molecules to sequentially knockout first PTB, then nPTB, thus efficiently converting a primate non-neural cell to a functional neuronal cell with mature neuronal marks.

Naturally occurring or synthetic nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids.

In some embodiments, the invention uses RNAi inhibitory nucleic acid molecules capable of decreasing or inhibiting expression of a primate PTB or nPTB gene, message or protein.

In one embodiment, the RNAi molecule comprises a double-stranded RNA (dsRNA) molecule. The RNAi molecule can comprise a double-stranded RNA (dsRNA) molecule, e.g., siRNA, miRNA (microRNA) and/or short hairpin RNA (shRNA) molecules. For example, in one embodiment, the invention uses inhibitory, e.g., siRNA, miRNA or shRNA, nucleic acids that inhibit or suppress the expression and/or activity of a primate PTB and/or nPTB.

In some embodiments, the RNAi is about 11, 12, 13, 14, 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi, e.g., siRNA for inhibiting transcription and/or miRNA to inhibit translation, is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, e.g., to silence one or a set of transcription factors responsible for maintaining the differentiated phenotype of the differentiated cell; see, e.g., Shuey (2002) Drug Discov. Today 7:1040-1046. In one aspect, the invention provides methods to selectively degrade an RNA using the RNAi's of the invention. In one aspect, the RNAi molecules of the invention can be used to generate a loss-of-function mutation in a cell. These processes may be practiced in vitro, ex vivo or in vivo.

In one embodiment, intracellular introduction of the RNAi (e.g., miRNA or siRNA) is by internalization of a target cell specific ligand bonded to an RNA binding protein comprising an RNAi (e.g., microRNA) is adsorbed. The ligand can be specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, e.g., U.S. Patent App. Pub. Nos. 20060030003; 20060025361; 20060019286; 20060019258. In one aspect, the invention provides lipid-based formulations for delivering, e.g., introducing nucleic acids of the invention as nucleic acid-lipid particles comprising an RNAi molecule to a cell, see .g., U.S. Patent App. Pub. No. 20060008910.

Methods for making and using RNAi molecules, e.g., siRNA and/or miRNA, for selectively degrade RNA are well known in the art, see, e.g., U.S. Pat. Nos. 6,506,559; 6,511,824; 6,515,109; 6,489,127. Methods for making expression constructs, e.g., vectors or plasmids, from which an inhibitory polynucleotide (e.g., a duplex siRNA of the invention) is transcribed are well known and routine. A regulatory region (e.g., promoter, enhancer, silencer, splice donor, acceptor, etc.) can be used to transcribe an RNA strand or RNA strands of an inhibitory polynucleotide from an expression construct. When making a duplex siRNA inhibitory molecule, the sense and antisense strands of the targeted portion of the targeted IRES can be transcribed as two separate RNA strands that will anneal together, or as a single RNA strand that will form a hairpin loop and anneal with itself. For example, a construct targeting a portion of a gene is inserted between two promoters such that transcription occurs bidirectionally and will result in complementary RNA strands that may subsequently anneal to form an inhibitory siRNA of the invention.

Alternatively, a targeted portion of a gene, coding sequence, promoter or transcript can be designed as a first and second antisense binding region together on a single expression vector; for example, comprising a first coding region of a targeted gene in sense orientation relative to its controlling promoter, and wherein the second coding region of the gene is in antisense orientation relative to its controlling promoter. If transcription of the sense and antisense coding regions of the targeted portion of the targeted gene occurs from two separate promoters, the result may be two separate RNA strands that may subsequently anneal to form a gene-inhibitory siRNA used to practice this invention.

In another embodiment, transcription of the sense and antisense targeted portion of the targeted gene is controlled by a single promoter, and the resulting transcript will be a single hairpin RNA strand that is self-complementary, i.e., forms a duplex by folding back on itself to create a gene-inhibitory siRNA molecule. In this configuration, a spacer, e.g., of nucleotides, between the sense and antisense coding regions of the targeted portion of the targeted gene can improve the ability of the single strand RNA to form a hairpin loop, wherein the hairpin loop comprises the spacer. In one embodiment, the spacer comprises a length of nucleotides of between about 5 to 50 nucleotides. In one aspect, the sense and antisense coding regions of the siRNA can each be on a separate expression vector and under the control of its own promoter.

In one embodiment, the nucleic acid molecule is an antisense oligonucleotide that targets a PTB pre-mRNA in order to induce exon skipping, and thereby to cause suppression of the expression or activity of a primate PTB protein in the non-neuronal cell. Exon skipping herein refers to the induction in a cell of a mature mRNA that does not contain a particular exon that is normally present therein. Exon skipping is achieved by providing a cell expressing the pre-mRNA of said mRNA with a molecule capable of interfering with sequences such as, for example, the splice donor or spliceacceptor sequence that are both required for allowing the enzymatic process of splicing, or a molecule that is capable of interfering with an exon inclusion signal required for recognition of a stretch of nucleotides as an exon to be included in the mRNA. The term pre-mRNA refers to a non-processed or partly processed precursor mRNA that is synthesized from a DNA template in the cell nucleus by transcription. Within the context of the invention inducing and/or promoting skipping of an exon as indicated herein means that at least 1%, 10%, 20%, 30%, 40%, 50%, 60%, 70%, 80%, 90% or more of the PTB mRNA in a non-neuronal cell will not contain said exon. This is preferably assessed by PCR as described in the examples.

In a preferred embodiment, a nucleic acid molecule that targets a target sequence or its complement in a primate PTB gene, transcript or mRNA is a guide RNA (gRNA) for a CRISPR/Cas family effector protein (Cas effector protein). In one embodiment therefore, the composition comprises a cell-programming agent comprising: i) a Cas effector protein and the at least one gRNA that targets a target sequence or its complement in a primate PTB gene, transcript or mRNA, or ii) at least one expression vector encoding a Cas effector protein and encoding the at least one gRNA. Likewise the invention provides for a composition comprising such cell-programming agents.

In some embodiment, the cell-programming agent (e.g. comprising the Cas effector protein and the at least one gRNA, or the at least one expression vector) is comprised in a nanoparticle, such as a liposome.

In one embodiment, a CRISPR/Cas family effector protein (Cas effector protein) and a guide RNA (gRNA) that targets a PTB gene sequence, the cell-programming agent comprises at least one expression vector encoding a Cas effector protein and encoding a gRNA that targets a PTB gene sequence.

In some embodiments, the Cas effector protein in the cell-programming agent is selected from the group consisting of: Cas13d, CasRx, Cas13e, CRISPR/Cas9, Cpf1, Cas9, Cas13a, Cas13b, Cas13c, Cas13f and a functional domain thereof. In certain embodiments, the Cas effector protein is encoded by an ORF (from start codon to stop codon) of 4.5 kb or less, 4 kb or less, 3.5 kb or less, 3 kb or less, 2.5 kb or less, or 2.1 kb or less, or 1.5 kb or less. In some embodiment, the Cas effector protein is modified to comprise a nuclear location signal. In some embodiment, the Cas effector protein is an DNA-targeting Cas effector protein. In some embodiment, the Cas effector protein is an DNA-targeting Cas effector protein selected from the group consisting of: spCas9 or its variant, SaCas9 or its variant, Cpf1 or its variant, and a combination thereof.

In certain embodiments, the Cas effector protein in the cell-programming agent is an RNA-targeting Cas effector protein. An RNA-targeting Cas effector protein is herein understood as a Cas effector protein that uses its crRNA/gRNA to recognize and degrade target RNA sequences, rather than target DNA sequences. In some embodiments, the Cas effector protein is the effector protein of a Type VI CRISPR-CAS system, preferably of a Type VI-D CRISPR-CAS system. In some embodiment, the Cas effector protein contains 2 HEPN ribonuclease motifs, containing the RXXXXH-motif (see Anantharaman et al., 2013, Biol Direct. 2013; 8: 15). In some embodiment, the RNA-targeting Cas effector protein is selected from the group consisting of: Cas13a, Cas13b, Cas13c, Cas13d/CasRx, CRISPR/Cas9, Cpf1, Cas13e and Cas13f and a functional domain thereof. In certain embodiments, the RNA-targeting Cas effector protein is encoded by an ORF (from start codon to stop codon) of 4.5 kb or less, 4 kb or less, 3.5 kb or less, 3 kb or less, 2.5 kb or less, or 2.1 kb or less, or 1.5 kb or less. In some embodiment, the RNA-targeting Cas effector protein is modified to comprise a nuclear location signal.

In a preferred embodiment, the RNA-targeting Cas effector protein is a Cas13d or an ortholog thereof, of which CasRx (described by Konermann et al., 2018, Cell 173, 665-676) is most preferred. CasRx is an ortholog of CRISPR-Cas13d, which has the smallest size and exhibits high targeting specificity and efficiency, making it a preferred option for in vivo therapeutic application.

A gRNA that targets a primate PTB mRNA sequence is preferably used in a cell-programming agent of the invention in combination with an RNA-targeting CRISPR/Cas family effector protein. Thus, in some embodiment, the gRNA comprises a sequence that is complementary to a target sequence in a primate PTB mRNA, such as e.g. the human PTBP1 coding sequence (NM_002819; SEQ ID NO: 87), preferably, the gRNA comprises a sequence that is complementary to a target sequence as herein defined above.

In one embodiment, the gRNA comprises a guide sequence that is complementary to a contiguous stretch of 14-60 nucleotides in a primate PTB mRNA sequence, such as SEQ ID NO: 87. Preferably, the gRNA comprises a guide sequence that is complementary to a contiguous stretch of at least 17 and no more than 60 nucleotides in a primate PTB mRNA sequence, such as SEQ ID NO: 87. In some certain embodiments, the gRNA comprises a guide sequence that is complementary to a contiguous stretch of at least 17, 18, 19, 20, 21, 22, 23, 24, 25, 26, 27, 28, 29 or 30 nucleotides in a primate PTB mRNA sequence, such as SEQ ID NO: 87, and/or no more than 60, 55, 50, 49, 48, 47, 46, 45, 44, 43, 42, 41 or 40 nucleotides in a primate PTB mRNA sequence, such as SEQ ID NO: 87. In some embodiments, the gRNA comprises a guide sequence that is complementary to a contiguous stretch of 25-45 nucleotides in a primate PTB mRNA sequence, such as SEQ ID NO: 87.

In one embodiment, the guide sequence in the gRNA comprises or consists of at least 10, 11, 12, 13, 14, 15, 16, 17 contiguous nucleotides, or of all nucleotides of at least one of SEQ ID NO.'s: 1-86.

In one embodiment, the guide sequence in the gRNA comprises or consists of at least 10, 11, 12, 13, 14, 15, 16, 17 contiguous nucleotides, or of all nucleotides of at least one of SEQ ID NO.'s: 1-86, wherein when the gRNA is co-expressed with CasRx in at least one of Cos7 and 293T cells, a relative expression of PTB mRNA of less than 0.5, 0.45, 0.4, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx.

In one embodiment, the guide sequence in the gRNA comprises or consists of at least one of SEQ ID NO.'s: 1-86. In one embodiment, the guide sequence in the gRNA comprises or consists of at least one of SEQ ID NO.'s: 1-86 and preferably produces a relative expression as determined in Table 1, that is no more than 0.5, 0.45, 0.4, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015.

In one embodiment, the guide sequence in the gRNA comprises or consists of at least one sequence selected from SEQ ID NO.'s: 56, 60, 47, 48, 43, 45, 40, 61, 50, 55, 41, 44, 42, 66, 49, 51, 46, 79, 07, 58, 53, 38, 64, 63, 59, 54, 62, 81, 83, 57, 05, 08, 70, 73, 76, 10, 04, 36, 52, 26, 67, 25, 39, 34, 80, 06, 23, 85, 24, 30, 02, 13, 03, 77, 31, 21, 69, 16, 75, 12, 78, 20, 74, 71 and 37. In one embodiment, the guide sequence in the gRNA comprises or consists of at least one sequence selected from SEQ ID NO.'s: 56, 60, 47, 48, 43, 45, 40, 61, 50, 55, 41, 44, 42, 66, 49, 51, 46, 79, 07, 58, 53, 38, 64, 63, 59, 54, 62, 81, 83, 57, 05, 08, 70, 73, 76, 10, 04, 36, 52, 26, 67, 25, 39, 34, 80, 06, 23, 85, 24, 30 and 02. In one embodiment, the guide sequence in the gRNA comprises or consists of at least one sequence selected from SEQ ID NO.'s: 56, 60, 47, 48, 43, 45, 40, 61, 50, 55, 41, 44, 42, 66, 49, 51, 46, 79, 07, 58, 53, 38, 64, 63, 59, 54, 62, 81, 83, 57, 05, 08, 70, 73, 76, 10, 04, 36, 52, 26, 67 and 25. In one embodiment, the guide sequence in the gRNA comprises or consists of at least one sequence selected from SEQ ID NO.'s: 56, 60, 47, 48, 43, 45, 40, 61, 50, 55, 41, 44, 42, 66, 49, 51, 46, 79, 07, 58, 53, 38, 64, 63, 59, 54, 62, 81, 83, 57, 05, 08, 70, 73 and 76. In one embodiment, the guide sequence in the gRNA comprises or consists of at least one sequence selected from SEQ ID NO.'s: 56, 60, 47, 48, 43, 45, 40, 61, 50, 55, 41, 44, 42, 66, 49, 51, 46, 79, 07, 58, 53, 38, 64, 63 and 59. In one embodiment, the guide sequence in the gRNA comprises or consists of at least one sequence selected from SEQ ID NO.'s: 56, 60, 47, 48, 43, 45, 40, 61, 50, 55, 41, 44, 42, 66, 49, 51 and 46. In one embodiment, the guide sequence in the gRNA comprises or consists of at least one sequence selected from SEQ ID NO.'s: 56, 60, 47, 48, 43, 45, 40, 61, 50, 55, 41, 44, 42, 66 and 49. In one embodiment, the guide sequence in the gRNA comprises or consists of at least one sequence selected from SEQ ID NO.'s: 56, 60, 47, 48, 43, 45, 40 and 61. In one embodiment, the guide sequence in the gRNA comprises or consists of at least one sequence selected from SEQ ID NO.'s: 56, 60, 47, 48, 43, 45 and 40. In one embodiment, the guide sequence in the gRNA comprises or consists of at least one sequence selected from SEQ ID NO.'s: 56, 60, 47, 48, 43 and 45. In one embodiment, the guide sequence in the gRNA comprises or consists of at least one sequence selected from SEQ ID NO.'s: 56, 60, 47, 48 and 43. In one embodiment, the guide sequence in the gRNA comprises or consists of at least one sequence selected from SEQ ID NO.'s: 56, 60, 47 and 48. In one embodiment, the guide sequence in the gRNA comprises or consists of at least one sequence selected from SEQ ID NO.'s: 56, 60 and 47. In one embodiment, the guide sequence in the gRNA comprises or consists of at least one sequence selected from SEQ ID NO.'s: 56 and 60.

In a preferred embodiment, the guide sequence in the gRNA comprises or consists of SEQ ID NO.: 60. In another preferred embodiment, the guide sequence in the gRNA comprises or consists of SEQ ID NO.: 56.

In certain embodiments, the gRNA targets a sequence in a primate PTB mRNA that corresponds to a sequence selected from the group consisting of: a) positions 59-91 of SEQ ID NO: 87; b) positions 303-349 of SEQ ID NO: 87; c) positions 422-451 of SEQ ID NO: 87; d) positions 460-489 of SEQ ID NO: 87; e) positions 542-576 of SEQ ID NO: 87; f) positions 646-681 of SEQ ID NO: 87; g) positions 706-769 of SEQ ID NO: 87; h) positions 773-806 of SEQ ID NO: 87; i) positions 1079-1139 of SEQ ID NO: 87; j) positions 1152-1184 of SEQ ID NO: 87; k) positions 1191-1254 of SEQ ID NO: 87; l) positions 1374-1434 of SEQ ID NO: 87; m) positions 951-1487 of SEQ ID NO: 87; n) positions 1502-1575 of SEQ ID NO: 87; and, o) positions 1626-1669 of SEQ ID NO: 87. It is herein understood that a sequence in a primate PTB mRNA (other than SEQ ID NO: 87) that corresponds to a sequence in positions with respect to SEQ ID NO: 87, is a sequence that corresponds to those positions in SEQ ID NO: 87 in a nucleotide sequence alignment, preferably using a global Needleman Wunsch algorithm using the default settings (default Gap opening penalty is 10.0 and the default gap extension penalty is 0.5; default scoring matrix DNAFull).

In some certain embodiments, the gRNA comprises or consists of a guide sequence that is complementary to a contiguous stretch of at least 17, 18, 19, 20, 21, 22, 23, 24, 25, 26, 27, 28, 29 or 30 nucleotides, and/or no more than 60, 55, 50, 49, 48, 47, 46, 45, 44, 43, 42, 41 or 40 nucleotides in a primate PTB mRNA sequence that is at least 95, 96, 97, 98, 99 or 100% identical to positions 951-1487 of SEQ ID NO: 87, and wherein preferably, when the target sequence is targeted with a guide RNA (gRNA), the guide sequence of which comprises or consists of at least one of SEQ ID NO.'s: 38-68, that is co-expressed with CasRx in at least one of Cos7 and 293T cells, a relative expression of PTB mRNA of less than 0.50, 0.45, 0.40, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx.

In one embodiment, the gRNA comprises or consists of a guide sequence that is complementary to a contiguous stretch of at least 17, 18, 19, 20, 21, 22, 23, 24, 25, 26, 27, 28, 29 or 30 nucleotides, and/or no more than 60, 55, 50, 49, 48, 47, 46, 45, 44, 43, 42, 41 or 40 nucleotides in a primate PTB mRNA sequence that is at least 95, 96, 97, 98, 99 or 100% identical to at least one of: positions 1000-1487 of SEQ ID NO: 87; positions 1050-1487 of SEQ ID NO: 87; positions 1100-1487 of SEQ ID NO: 87; positions 1150-1487 of SEQ ID NO: 87; positions 1200-1487 of SEQ ID NO: 87; positions 1250-1487 of SEQ ID NO: 87; positions 1300-1487 of SEQ ID NO: 87; positions 1350-1487 of SEQ ID NO: 87; positions 1400-1487 of SEQ ID NO: 87; positions 951-1400 of SEQ ID NO: 87; positions 951-1350 of SEQ ID NO: 87; positions 951-1300 of SEQ ID NO: 87; positions 951-1250 of SEQ ID NO: 87; positions 951-1200 of SEQ ID NO: 87; positions 951-1150 of SEQ ID NO: 87; positions 951-1100 of SEQ ID NO: 87; positions 951-1050 of SEQ ID NO: 87; and positions 951-1000 of SEQ ID NO: 87; and wherein preferably, when the target sequence is targeted with a guide RNA (gRNA), the guide sequence of which comprises or consists of at least one of SEQ ID NO.'s: 38-68, that is co-expressed with CasRx in at least one of Cos7 and 293T cells, a relative expression of PTB mRNA of less than 0.50, 0.45, 0.40, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx.

In some embodiments, co-expression of a gRNA of the invention together with CasRx in a human or non-human primate cell (e.g. in at least one of Cos7 and 293T cells) causes a relative expression of PTB mRNA of no more than 0.9, 0.85, 0.8, 0.75, 0.7, 0.65, 0.6, 0.55, 0.5, 0.45, 0.4, 0.35, 0.3, 0.25, 0.24, 0.23, 0.22, 0.21, 0.20, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02 or 0.01 as compared to a corresponding control cell expressing only CasRx. The relative expression of a gRNA of the invention can be assayed as described in the Examples herein. For example, transient co-transfection can be performed with 4 μg vectors expressing CAG-CasRx-P2A-GFP and 2 μg U6-gRNA-CMV-mCherry plasmid using Lipofectamine 3000 (or similar transfection reagent). Cell transfected with only CAG-CasRx-P2A-GFP plasmid can be used as a control. Two days after transient transfection, around 30K GFP and mCherry double-positive (GFP top 20%) cells are collected by fluorescence activated cell sorting (FACS) and lysed for qPCR analysis to determine the relative expression of PTB mRNA compared to a corresponding amount of controls cells (sorted for GFP top 20% only).

As provided herein, a cell-programming agent (e.g., Cas with PTB-targeting gRNA or polynucleotide encoding the same) suppresses expression or activity of PTB by at least about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the endogenous or native level. As provided herein, cell-programming agent (e.g., Cas with nPTB-targeting gRNA or polynucleotide encoding the same) suppresses expression or activity of nPTB by about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the endogenous or native level.

In some embodiments, a cell-programming agent as provided herein (e.g., Cas with PTB/nPTB-targeting gRNA or polynucleotide encoding the same) directly suppress the expression level of PTB/nPTB, e.g., suppressing the transcription, translation, or protein stability of PTB and/or nPTB.

In some embodiments, a cell-programming agent as provided herein (e.g., Cas with PTB/nPTB-targeting gRNA or polynucleotide encoding the same) directly effects on the expression or activity of PTB/nPTB, without affecting other cellular signaling pathway.

In some embodiments, the composition comprising the cell-programming agent comprises at least one gRNA that targets a primate PTB mRNA sequence or the at least one expression vector encodes at least one gRNA that targets a primate PTB mRNA sequence. In another embodiment, the composition comprising the cell-programming agent comprises no more than one (type of) gRNA that targets a primate PTB mRNA sequence or the at least one expression vector encodes no more than one (type of) gRNA that targets a primate PTB mRNA sequence. In yet another embodiment, the composition comprising the cell-programming agent comprises two, three, four five or six different (types of) gRNAs that target a primate PTB mRNA sequence or the at least one expression vector encodes two, three, four five or six different (types of) gRNAs that target a primate PTB mRNA sequence.

As provided herein, contacting the non-neuronal cell with a composition comprising a cell-programming agent as provided herein, can be performed in any appropriate manner, depending on the type of non-neuronal cell to be reprogrammed, the environment in which the non-neuronal cell resides, and the desired cell reprogramming outcome.

In some embodiments, the non-neuronal cell is contacted with a composition comprising a cell-programming agent as provided herein in the form of a polynucleotide encoding the encoding the Cas with PTB/nPTB-targeting gRNA. Thus, in one embodiment, the non-neuronal cell is contacted with a composition comprising a cell-programming agent as provided herein in the form of at least one expression vector encoding a Cas effector protein and encoding a gRNA that targets a primate PTB mRNA sequence.

In some embodiments, the at least one expression vector comprises a nucleotide sequence encoding the Cas effector protein that is operably linked to a promoter that causes expression of the Cas effector protein in non-neuronal cell. In the some embodiments, the promoter that causes expression in non-neuronal cell is a glial cell-specific promoter or a Müller glia (MG) cell-specific promoter. In some embodiments, the glial cell-specific promoter is selected from the group consisting of: the GFAP promoter, the ALDH1L1 promoter, the EAAT1/GLAST promoter, the glutamine synthetase promoter, S100 beta promoter and the EAAT2/GLT-1 promoter. In some embodiments, the MG cell-specific promoter is selected from the group consisting of: the GFAP promoter, the ALDH1L1 promoter, Glast (also known as Slc1a3) promoter and the Rlbp1 promoter. In some embodiments, the glial cell-specific promoter or Müller glia (MG) cell-specific promoter is a primate, human or non-human primate promoter.

In some embodiments, the at least one expression vector comprises at least one nucleotide sequence encoding a gRNA that targets a primate PTB mRNA sequence, which nucleotide sequence is operably linked to a promoter that causes expression of the gRNA in the non-neuronal cell. In some embodiments, the promoter that is operably linked to a gRNA coding sequence (and that causes expression of the gRNA in the non-neuronal cell) is a promoter from a U6 snRNA gene, preferably a primate, human or non-human primate U6 promoter.

In some embodiments, nucleotide sequence encoding the Cas effector protein is adapted to optimize their codon usage to that of the primate, human or non-human primate (non-neuronal) host cell. The adaptiveness of a nucleotide sequence encoding a polypeptide to the codon usage of a host cell may be expressed as codon adaptation index (CAI). The codon adaptation index is herein defined as a measurement of the relative adaptiveness of the codon usage of a gene towards the codon usage of highly expressed genes in a particular host cell or organism. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al., 2003, Nucleic Acids Res. 31(8):2242-51). An adapted nucleotide sequence encoding the Cas effector protein preferably has a CAI of at least 0.2, 0.3, 0.4, 0.5, 0.6, 0.7, 0.8 or 0.9. Codon optimization methods for optimum gene expression in heterologous organisms are known in the art and have been previously described (see e.g., Welch et al., 2009, PLoS One 4:e7002; Gustafsson et al., 2004, Trends Biotechnol. 22:346-353; Wu et al., 2007, Nucl. Acids Res. 35:D76-79; Villalobos et al., 2006, BMC Bioinformatics 7:285; U.S. Patent Publication 2011/0111413; and U.S. Patent Publication 2008/0292918).

In these configurations, non-viral transfection methods or viral transduction methods are utilized to introduce the cell-programming agent. Non-viral transfection can refer to all cell transfection methods that are not mediated through a virus. Non-limiting examples of non-viral transfection include electroporation, microinjection, calcium phosphate precipitation, transfection with cationic polymers, such as DEAE-dextran followed by polyethylene glycol, transfection with dendrimers, liposome mediated transfection (“lipofection”), microprojectile bombardment (“gene gun”), fugene, direct sonic loading, cell squeezing, optical transfection, protoplast fusion, impalefection, magnetofection, nucleofection, and any combination thereof.

In some embodiments, the methods provided herein utilize gene therapy vectors, such as viral vectors as appropriate medium for delivering the cell-programming agent to the non-neuronal cell. As provided herein, viral vector methods can include the use of either DNA or RNA viral vectors. Examples of appropriate viral vectors can include adenoviral, lentiviral, adeno-associated viral (AAV), poliovirus, herpes simplex virus (HSV), or murine Maloney-based viral vectors.

In some embodiments, the vector is an AAV vector. In some embodiments, a cell-programming agent is administered in the form of AAV vector. In some embodiments, a cell-programming agent is administered in the form of lentiviral vector. For example, a cell-programming agent can be delivered to a non-neuronal cell using a lentivirus or adenovirus associated virus (AAV) to express a Cas effector protein with gRNA against PTB/nPTB.

According to some embodiments of the disclosure, methods provided herein comprise suppressing the expression or activity of PTB/nPTB in a non-neuronal cell (e.g., a glia cell or astrocyte) via a cell-programming agent of a sufficient amount for reprogramming the non-neuronal cell to a mature neuron. The sufficient amount of cell-programming agent can be determined empirically as one skilled in the art would readily appreciate. In some embodiments, the amount of cell-programming agent can be determined by any type of assay that examines the activity of the cell-programming agent in the non-neuronal cell.

For example, when the cell-programming agent is configured to suppress the expression of PTB/nPTB in the non-neuronal cell, the sufficient amount of the cell-programming agent can be determined by assessing the expression level of PTB/nPTB in an exemplary non-neuronal cell after administration of the agent, e.g., by Western blot. In some embodiments, functional assays are utilized for assessing the activity of PTB/nPTB after delivery of the cell-programming agent to an exemplary non-neuronal cell. In some embodiments, other functional assays, such as, immunostaining for neuronal markers, electrical recording for neuronal functional properties, that examine downstream neuronal properties are used to determine a sufficient amount of cell-programming agent.

In some embodiments, the cell-programming agent is delivered in the form of a viral vector. A viral vector can comprises one or more copies of expression sequence coding for a cell-programming agent, e.g., a Cas effector protein with one or more copies of coding sequence for gRNA against PTB/nPTB, such as, 1, 2, 3, 4, 5, 6, 7, 8, 9, 10 copies.

A viral vector can be tittered to any appropriate amount for administration, as one skilled in the art will be able to determine. For example, the titer as determined by PCR, RT-PCR, or other methods can be at least about 10⁵ viral particles/mL, 10⁶ particles/mL, 10⁷ particles/mL, 10⁸ particles/mL, 10⁹ particles/mL, 10¹⁰ particles/mL, 10¹¹ particles/mL, 10¹² particles/mL, 10¹³ particles/mL, 10¹⁴ particles/mL, or 10¹⁵ particles/mL.

In some embodiments, the titer of viral vector to be administered is at least about 10¹⁰ particles/mL, 10¹¹ particles/mL, 10¹² particles/mL, 10¹³ particles/mL, or 10¹⁴ particles/mL.

In other embodiment of the invention, a composition comprising the PTB-targeting cell-programming agent as defined above, comprises additional components, such as one or more non-PTB-targeting cell-programming agents, to further increase neuron conversion efficiency. The combination of the PTB-targeting cell-programming agent with one or more non-PTB-targeting cell-programming agents can act synergistically in increasing neuron conversion efficiency.

Thus, in one embodiment, a composition comprising PTB-targeting cell-programming agent as defined above, further comprises i) one or more dopamine neuron-associated factors, and/or ii) at least one expression vector for expression of one or more dopamine neuron-associated factors, preferably in a non-neuronal cell. Preferably, such a composition comprising both the PTB-targeting cell-programming agent and the dopamine neuron-associated factor is for local administration to a non-neuronal cell in the striatum. More preferably, the composition is administered to a to a non-neuronal cell in the striatum for generating a functional dopaminergic neuron, whereby it is further preferred that the composition is administered to a glial cell in the striatum for generating a functional dopaminergic neuron.

In one embodiment, the one or more dopamine neuron-associated factors are selected from the group consisting of: Lmx1a, Lmx1b, FoxA2, Nurr1, Pitx3, Gata2, Gata3, FGF8, BMP, En1, En2, PET1, a Pax family protein, SHH, a Wnt family protein and a TGF-β family protein. In one embodiment, the one or more dopamine neuron-associated factors are selected from the group consisting of: FoxA2, Lmx1a and Nurr1. In a preferred embodiment, the one or more dopamine neuron-associated factors are FoxA2 alone, Lmx1a alone, Nurr1 alone, the combination of FoxA2 and Lmx1a, the combination of FoxA2 and Nurr1, the combination of Nurr1 and Lmx1a or the combination of FoxA2, Lmx1a and Nurr1. Suitable primate and human amino acid and/or nucleotide sequences of these dopamine neuron-associated factors/genes are known to the skilled person and can be accessed in publicly available databases. Construction and delivery of expression vectors for the expression of the one or more dopamine neuron-associated factors in the non-neuronal cell can be as described herein above for expression vectors of the PTB-targeting cell-programming agent.

In another embodiment, a composition comprising PTB-targeting cell-programming agent as defined above, further comprises i) one or more factors selected from β-catenin, OSK factors (also known as Oct4, Sox2, Klf4 and other factors involved in the cell re-cycling or epigenetic remodeling), Crx, Brn3a, Brn3b, Math5, Nr2e3 and Nrl, and/or ii) at least one expression vector for expression of one or more factors selected from β-catenin, OSK factors (also known as Oct4, Sox2, Klf4 and other factors involved in the cell re-cycling or epigenetic remodeling), Crx, Brn3a, Brn3b, Math5, Nr2e3 and Nrl, preferably in a non-neuronal cell. In one embodiment, the one or more factors are selected from the group consisting of: β-catenin and the OSK factors, Oct4, Sox2 and Klf4. In a preferred embodiment, the one or more factors are a combination of β-catenin and Oct4, a combination of β-catenin and Sox2, a combination of β-catenin and Klf4, a combination of β-catenin and Oct4 with Sox2, a combination of β-catenin and Oct4 with Klf4, a combination of β-catenin and Sox2 with Klf4, a combination of β-catenin and all three OSK factors, Oct4, Sox2 and Klf4, or a combination of all three OSK factors, Oct4, Sox2 and Klf4 (without β-catenin). Preferably, such a composition comprising both the PTB-targeting cell-programming agent and the factor is for local administration to a non-neuronal cell in a mature retina. More preferably, the composition is administered to a to a non-neuronal cell in the mature retina for generating a functional retinal ganglion cell (RGC) neuron and/or a functional retinal photoreceptor, whereby it is further preferred that the composition is administered to a glial cell or Müller glia (MG) cell in the mature retina for generating a functional retinal ganglion cell (RGC) neuron and/or a functional retinal photoreceptor. In a preferred embodiment, the factor is β-catenin. Suitable primate and human amino acid and/or nucleotide sequences of β-catenin, Oct4, Sox2, Klf4, Crx, Brn3a, Brn3b, Math5, Nr2e3 and Nrl or their genes are known to the skilled person and can be accessed in publicly available databases. Construction and delivery of expression vectors for the expression of the one or more factors selected from β-catenin, Oct4, Sox2, Klf4, Crx, Brn3a, Brn3b, Math5, Nr2e3 and Nrl in the non-neuronal cell can be as described herein above for expression vectors of the PTB-targeting cell-programming agent.

### Dosing and Treatment Regimens

Methods provided herein can comprise suppressing the expression or activity of PTB/nPTB in a non-neuronal cell for a certain period of time sufficient for reprogramming the non-neuronal cell to a mature neuron.

In some embodiments, exemplary methods comprise contacting the non-neuronal cell with a cell-programming agent that suppresses the expression or activity of PTB/nPTB in the non-neuronal cell for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 3 weeks, at least 4 weeks, at least 5 weeks, at least 2 months, at least 3 months, at least 4 months, at least 5 months, or at least 1 year, thereby reprogramming the non-neuronal cell to a mature neuron.

In certain embodiments, suppression of PTB and nPTB expression or activity is sequential. For example, the expression or activity of PTB is first suppressed for, e.g., any one of the above-mentioned time period, before the expression or activity of nPTB is suppressed.

In certain embodiments, suppression of PTB and nPTB expression or activity is concurrent.

In some embodiments, exemplary methods comprise contacting the non-neuronal cell with a cell-programming agent that suppresses the expression or activity of PTB in the non-neuronal cell for about 1 day, 2 days, 3 days, 4 days, 5 days, 6 days, 7 days, 8 days, 9 days, 10 days, 11 days, 12 days, 13 days, 14 days, 15 days, 3 weeks, 4 weeks, 5 weeks, 2 months, 3 months, 4 months, or 5 months, before contacting the non-neuronal cell with a cell-programming agent that suppresses the expression or activity of nPTB in the non-neuronal cell, thereby reprogramming the non-neuronal cell to a mature neuron.

In some configurations, the methods provided herein comprise administering the cell-programming agent for only once, e.g., adding the cell-programming agent to a cell culture comprising the non-neuronal cell, or delivering the cell-programming agent to a brain region comprising the non-neuronal cell (e.g., striatum), for only once, and the cell-programming agent can remain active as suppressing expression or activity of PTB/nPTB in the non-neuronal cell for a desirable amount of time, e.g., for at least 1 day, at least 2 days, at least 4 days, or at last 10 days. For instance, when the cell-programming agent comprises an AAV vector expressing a Cas effector and a coding sequence for an anti-PTB gRNA, the design of the AAV vector can enable it to remain transcriptionally active for an extended period of time.

In some embodiments, the methods provided herein comprise administering the cell-programming agent for more than once, e.g., for at least 2 times, at least 3 times, at least 4 times, at least 5 times, at least 6 times, at least 7 times, at least 8 times, at least 9 times, at least 10 times, at least 12 times, at least 15 times, at least 20 times or more.

In some embodiments, the methods provided herein further comprise administering at least one immunosuppressant. In one embodiment, the immunosuppressant is administered prior to, simultaneously with, and/or after the administration of the cell-programming agent, for example, when the cell-programming agent comprises an AAV vector expressing a Cas effector and a coding sequence for an anti-PTB gRNA. Suitable immunosuppressants include e.g. corticosteroids (e.g. prednisone, prednisolone, dexamethasone, etc), calcineurin inhibitors (e.g. cyclosporine, tacrolimus, etc), mTOR inhibitors (sirolimus, everolimus, etc), IMDH inhibitors (azathioprine, mycophenolate, etc), antibodies (e.g. basiliximab, rituximab, alemtuzumab, etc), interferons (e.g. IFN-γ, etc), Janus kinase inhibitors (e.g. tofacitinib, etc) and biologics (anakinra, etc). In one embodiment, the immunosuppresant is administered about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 15 days, 3 weeks, 4 weeks, 5 weeks, 2 months, 3 months, 4 months, or 5 months, before and/or after administration of the cell-programming agent. In one embodiment, the immunosuppresant is administered about simultaneously with the administration of the cell-programming agent.

### Markers

According to some embodiments of the present disclosure, the methods provided herein comprise reprogramming a plurality of non-neuronal cells into mature neurons at a high efficiency.

In some embodiments, the methods comprise reprogramming MG cells or astrocytes into mature neurons, and at least 60% of the MG cells/astrocytes are converted to mature neurons that are Map2 or NeuN positive.

In some embodiments, at least 40% of the astrocytes are converted to mature neurons that are Map2 positive. In some embodiments, at least about 20%, 25%, 30%, 35%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, 99%, or 100% of the MG cells/astrocytes are converted to mature neurons that are positive for NeuN or Map2.

In some embodiments, at least 10 MG cells are converted to RGCs which express Brn3a or Rbpms in retinal ganglion cell layer (GCL) per 10 mm×50 μm. In some embodiments, at least about 1, 2, 4, 8, 10, 12, 14, 16, 18, 20, 22, 24, 26, 28, 30, 32, 34, 36, 38, 40, 42, 44, 46, 48, 50, 52, 54, 56, 58, 60 MG cells are converted to RGCs which express Brn3a or Rbpms in retinal ganglion cell layer (GCL) per 10 mm×50 μm.

In some embodiments, the methods comprise reprogramming human astrocytes into mature neurons, and at least 40%, at least 60%, or at least 80% of the human astrocytes are converted to mature neurons that are Map2 or NeuN positive. In some embodiments, at least 20%, at least 40% or at least 60% of the human astrocytes are converted to mature neurons that are Map2 or NeuN positive. In some embodiments, at least about 20%, 25%, 30%, 35%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, 99%, or 100% of the human astrocytes are converted to mature neurons that are positive for Map2 or NeuN.

In some embodiments, the methods as provided herein comprise reprogramming a plurality of non-neuronal cells, e.g., human non-neuronal cells, e.g., human glial cells, or astrocytes, and at least about 20%, 25%, 30%, 35%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 99% of the non-neuronal cells, e.g., human non-neuronal cells, e.g., human glial cells, or astrocytes are reprogrammed to mature neurons. In some embodiments, the methods as provided herein reprogram about 20%, 25%, 30%, 35%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92% 94%, 96% 98% 99%, or 100% of the non-neuronal cells, e.g., human non-neuronal cells, e.g., human glial cells, MG cells, or astrocytes are reprogrammed to mature neurons.

In some embodiments, a mature neuron is characterized by its expression of one or more neuronal markers selected from the group consisting of NeuN (neuronal nuclei antigen), Map2 (microtubule-associated protein 2), NSE (neuron specific enolase), 160 kDa neurofilament medium, 200 kDa neurofilament heavy, PDS-95 (postsynaptic density protein 95), Synapsin I, Synaptophysin, GAD67 (glutamate decarboxylase 67), GAD65 (glutamate decarboxylase 67), parvalbumin, DARPP32 (dopamine- and cAMP-regulated neuronal phosphoprotein 32), vGLUT1 (vesicular glutamate transporter 1), vGLUT2 (vesicular glutamate transporter 2), acetylcholine, vesicular GABA transporter (VGAT), and gamma-aminobutyric acid (GABA), and TH (tyrosine hydroxylase). In some embodiments, at least 40% of the non-neuronal cells, e.g., human non-neuronal cells, e.g., human glial cells, or astrocytes are reprogrammed to mature neurons.

In some embodiments, MG cells are converted into retinal photoreceptors characterized by expression of one or more rod cell markers selected from rhodopsin and GNAT1, and/or characterized by expression of one or more cone cell markers selected from S-opsin, M-opsin and mCAR.

As one of ordinary skills in the art would readily appreciate, the expression of all those markers above can be assessed by any common techniques. For examples, immunostaining using antibodies against specific cell type markers as described herein can reveal whether or not the cell of interest expresses the corresponding cell type marker. Immunostaining under certain conditions can also uncover the subcellular distribution of the cell type marker, which can also be important for determining the developmental stage of the cell of interest. For instance, expression of Map2 can be found in various neurites (e.g., dendrites) in a post-mitotic mature neuron, but absent in axon of the neuron. Expression of voltage-gated sodium channels (e.g., a subunits Navi.1-1.9) and b subunits) can be another example, they can be clustered in a mature neuron at axon initial segment, where action potential can be initiated, and Node of Ranvier. In some embodiments, other techniques, such as, but not limited to, flow cytometry, mass spectrometry, in situ hybridization, RT-PCR, and microarray, can also be used for assessing expression of specific cell type markers as described herein.

### Conversion Efficiency

Certain aspects of the present disclosure provide methods that comprise reprogramming a plurality of non-neuronal cells, and at least about 20%, 25%, 30%, 35%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 99% of the non-neuronal cells, e.g., human non-neuronal cells, e.g., human glial cells, MG cells, or astrocytes are reprogrammed to functional neurons.

In some embodiments, the methods provided herein reprogram at least 20% of the non-neuronal cells, e.g., human non-neuronal cells, e.g., human glial cells, MG cells, or astrocytes are reprogrammed to functional neurons. In some embodiments, the methods provided herein reprogram about 20%, 25%, 30%, 35%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90% 92% 94%, 96%, 98%, 99%, or 100% of the non-neuronal cells, e.g., human non-neuronal cells, e.g., human glial cells, MG cells, or astrocytes are reprogrammed to functional neurons.

In some embodiments, wherein the methods provided herein comprise the use of a combination of i) a PTB-targeting cell-programming agent as defined herein, and ii) one or more non-PTB-targeting cell-programming agents as defined herein, the combination increases the neuron conversion efficiency by at least a factor 1.1, 1.2, 1.5, 2.0, 3.0, 4.0, 5.0, 8.0, 10, 15 or 20.

### Functional Assessment

In some embodiments, functional neurons are characterized in their ability to form neuronal network, to send and receive neuronal signals, or both. In some embodiments, functional neurons fire action potential. In some embodiments, functional neurons establish synaptic connections with other neurons. For instance, a functional neuron can be a postsynaptic neuron in a synapse, e.g., having its dendritic termini, e.g., dendritic spines, forming postsynaptic compartments in synapses with another neuron. For instance, a functional neuron can be a presynaptic neuron in a synapse, e.g., having axonal terminal forming presynaptic terminal in synapses with another neuron.

Synapses a functional neuron can form with another neuron can include, but not limited to, axoaxonic, axodendritic, and axosomatic. Synapses a functional neuron can form with another neuron can be excitatory (e.g., glutamatergic), inhibitory (e.g., GABAergic), modulatory, or any combination thereof. In some embodiments, synapses a functional neuron forms with another neuron is glutamatergic, GABAergic, cholinergic, adrenergic, dopaminergic, or any other appropriate type. As a presynaptic neuron, a function neuron can release neurotransmitter such as, but not limited to, glutamate, GABA, acetylcholine, aspartate, D-serine, glycine, nitric oxide (NO), carbon monoxide (CO), hydrogen sulfide (H2S), dopamine, norepinephrine (also known as noradrenaline), epinephrine (adrenaline), histamine, serotonin, phenethylamine, N-methylphenethylamine, tyramine, 3-iodothyronamine, octopamine, tryptamine, somatostatin, substance P, opioid peptides, adenosine triphosphate (ATP), adenosine, and anandamide. As a postsynaptic neuron, a functional neuron can elicit postsynaptic response to a neurotransmitter released by a presynaptic neuron into the synaptic cleft. The postsynaptic response a functional neuron as generated in the method provided herein can be either excitatory, inhibitory, or any combination thereof, depending on the type of neurotransmitter receptor the functional neuron expresses. In some embodiments, the functional neuron expresses ionic neurotransmitter receptors, e.g., ionic glutamate receptors and ionic GABA receptors. Ionic glutamate receptors can include, but not limited to, a-amino-3-hydroxy-5-methyl-4-isoxazolepropionic acid (AMPA)-type glutamate receptors (e.g., GluAl/GRIAI; GluA2/GRIA2; GluA3/GRIA3; GluA4/GRIA4), delta receptors (e.g., GluDI/GRIDI; GluD2/GRID2), kainate receptors (e.g., GluKI/GRIKI; GluK2/GRIK2; GluK3/GRIK3; GluK4/GRIK4; GluK5/GRIK5) and iV-methyl-D-aspartate (NMDA) receptors (e.g., GluNI/GRINI; GluN2A/GRIN2A; GluN2B/GRIN2B; GluN2C/GRIN2C; GluN2D/GRIN2D; GluN3A/GRIN3A; GluN3B/GRIN3B). Ionic GABA receptors can include, but not limited to, GABAA receptor. In some embodiments, the functional neuron expresses metabolic neurotransmitter receptors, e.g., metabolic glutamate receptors (e.g., mGluRi, mGluRs, mGluR, mGluR, mGluRi, mGluRe, mGluR, mGluRs), and metabolic GABA receptors (e.g., GABAB receptor). In some embodiments, the functional neuron expresses a type of dopamine receptor, either DI-like family dopamine receptor, e.g., D1 and D5 receptor (DIR and D5R), or D2-like family dopamine receptor, e.g., D2, D3, and D4 receptors (D2R, D3R, and D4R). In some embodiments, a functional neuron as provided herein forms electrical synapse with another neuron (e.g., gap junction). In some embodiments, a function neuron as provided herein forms either chemical or electrical synapse (s) with itself, as known as autapse.

The characteristics of a function neuron can be assessed by common techniques available to one skilled in the art. For example, the electrical properties of a functional neuron, such as, firing of action potential and postsynaptic response to neurotransmitter release can be examined by techniques such as patch clamp recording (e.g., current clamp and voltage clamp recordings), intracellular recording, and extracellular recordings (e.g., tetrode recording, single-wire recording, and filed potential recording). Specific properties of a functional neuron (e.g., expression of ion channels and resting membrane potential) can also be examined by patch clamp recording, where different variants of patch clamp recording can be applied for different purposes, such as cell-attached patch, inside-out patch, outside-out patch, whole-cell recording, perforated patch, loose patch. Assessment of postsynaptic response by electrical methods can be coupled with either electrical stimulation of presynaptic neurons, application of neurotransmitters or receptor agonists or antagonists. In some cases, AMPA-type glutamate receptor-mediated postsynaptic current can be assessed by AMPA receptor agonists, e.g., AMPA, or antagonists, e.g., 2, 3-dihydroxy-6-nitro-7-sulfamoyl-benzoquinoxaline (NBQX) or 6-cyano-7-nitroquinoxaline-2,3-dione (CNQX). In some cases, NMDA-type glutamate receptor-mediated postsynaptic current can be assessed by NMDA receptor agonists, e.g., NMDA and glycine, or antagonists, e.g., AP5 and ketamine.

In some embodiments, functional neurons are examined by techniques other than electrical approaches. For example, recent development of various fluorescent dyes or genetically encoded fluorescent proteins and imaging techniques can be utilized for monitoring electrical signals conveyed or transmitted by a functional neuron. In this context, calcium-dependent fluorescent dyes (e.g., calcium indicators), such as, but not limited to, fura-2, indo-1, fluo-3, fluo-4, and Calcium Green-1, and calcium-dependent fluorescent proteins, such as, but not limited to, Cameleons, FIP-CBSM, Pericams, GCaMP, TN-L15, TN-humTnC, TN-XL, TNXXL, and Twitch's, can be used to trace calcium influx and efflux as an indicator of neuronal membrane potential. Alternatively or additionally, voltage-sensitive dyes that can change their spectral properties in response to voltage changes can also be used for monitoring neuronal activities.

Neurotransmitter release can be an important aspect of a functional neuron. The methods provided herein can comprise reprogram a non-neuronal cell to a functional neuron that releases a certain type of neurotransmitter. In some embodiments, the functional neuron releases neurotransmitter such as, but not limited to, glutamate, GABA, acetylcholine, aspartate, D-serine, glycine, nitric oxide (NO), carbon monoxide (CO), hydrogen sulfide (H2S), dopamine, norepinephrine (also known as noradrenaline), epinephrine (adrenaline), histamine, serotonin, phenethylamine, N-methylphenethylamine, tyramine, 3-iodothyronamine, octopamine, tryptamine, somatostatin, substance P, opioid peptides, adenosine triphosphate (ATP), adenosine, and anandamide.

In some embodiments, the functional neuron releases dopamine as a major neurotransmitter. In some embodiments, the functional neuron releases more than one type of neurotransmitter. In some embodiments, the functional neuron releases neurotransmitter in response to an action potential. In some embodiments, the functional neuron releases neurotransmitter in response to graded electrical potential (e.g., membrane potential changes that do not exceed a threshold for eliciting an action potential). In some embodiments, the functional neuron exhibits neurotransmitter release at a basal level (e.g., spontaneous neurotransmitter release). Neurotransmitter release as described herein from a functional neuron can be assessed by various techniques that are available to one of ordinary skills in the art. In some embodiments, imaging approaches can be used for characterizing a functional neuron's neurotransmitter release, for instance, by imaging a genetically encoded fluorescent fusion molecule comprising a vesicular protein, one can monitor the process of synaptic vesicles being fused to presynaptic membrane.

Alternatively or additionally, other methods can be applied to directly monitor the level of a specific neurotransmitter. For example, HPLC probe can be used to measure the amount of dopamine in a culture dish or a brain region where a functional neuron projects its axon to. The level of dopamine as detected by HPLC can indicate the presynaptic activity of a functional neuron. In some embodiments, such assessment can be coupled with stimulation of the functional neuron, in order to change its membrane potential, e.g., to make it elicit action potential.

In an aspect, the present disclosure provides a method of generating a functional neuron in vivo. An exemplary method comprises administering to a region in the nervous system, e.g., mature retina or inner ear or a region in the brain or spinal cord (e.g., striatum), of a subject a composition comprising a cell-programming agent (e.g., a Cas effector protein and a gRNA targeting/complementing to PTB and/or nPTB, or polynucleotide encoding the same) in a non-neuronal cell (e.g., a glial cell or astrocyte) in the region in nervous system, and allowing the non-neuronal cell to reprogram into the functional neuron. In some embodiments, the cell-programming agent suppresses the expression or activity of PTB and/or nPTB.

### Administration Route

According to some embodiments of the present disclosure, the methods provided herein comprise direct administration of a cell-programming agent (e.g., a Cas effector protein and a gRNA targeting/complementing to PTB and/or nPTB or polynucleotide encoding the same) into a region in the nervous system (e.g., mature retina or inner ear or a region in the brain or spinal cord (e.g., striatum) of a subject. In some embodiments, the cell-programming agent (e.g., a Cas effector protein and a gRNA targeting/complementing to PTB and/or nPTB or polynucleotide encoding the same) is delivered locally to a region in the nervous system (e.g., mature retina or a region in the brain or spinal cord (e.g., striatum)). In one embodiment, a composition comprising a cell-programming agent, such as a viral vector (e.g. AAV vector), is administered to the subject or organism by stereotaxic or convection enhanced delivery to a brain region (e.g., striatum).

Using stereotaxic positioning system, one skilled in the art would be able to locate a specific brain region (e.g., striatum) that is to be administered with the composition comprising the cell-programming agent. Such methods and devices can be readily used for the delivery of the composition as provided herein to a subject or organism. In another embodiment, a composition as provided herein is delivered systemically to a subject or to a region in nervous system, e.g., brain (e.g., striatum) or spinal cord, of a subject, e.g., delivered to cerebrospinal fluid or cerebral ventricles, and the composition comprises one or more agents that are configured to relocate the cell-programming agent to a particular region in the nervous system (e.g., striatum) or a particular type of cells in the nervous system of the subject.

In some embodiments, the cell-programming agent used in the methods provided herein comprises a virus that expresses a Cas effector and an anti-PTB or anti-nPTB gRNA, and the methods comprise injection of the virus in a desired brain region stereotaxically. In some embodiments, the virus comprises adenovirus, lentivirus, adeno-associated virus (AAV), poliovirus, herpes simplex virus (HSV), or murine Maloney-based virus. The AAV that can be used in the methods provided herein can be any appropriate serotype of AAV, such as, but not limited to, AAV2, AAV5, AAV6, AAV7, AAV8, and AAV9. In some embodiments, the methods comprise delivering an AAV2- or AAV9-based viral vector that expresses an agent that suppresses expression or activity of PTB and/or nPTB in a non-neuronal cell in a region in nervous system, e.g., brain (e.g., striatum) or spinal cord.

In some embodiments, as described above, the methods provided herein comprise reprogramming a variety of non-neuronal cells to mature neurons. In some embodiments, the methods provided herein comprise administering to a region in the nervous system, e.g., brain (e.g., striatum) or spinal cord, of a subject a composition comprising a cell-programming agent that suppresses the expression or activity of PTB and/or nPTB in a variety of non-neuronal cells, such as, but not limited to, glial cells, e.g., astrocyte, oligodendrocyte, NG2 cell, satellite cell, or ependymal cell in the nervous system, and allowing the non-neuronal cell to reprogram into the functional neuron. In some embodiments, the methods provided herein comprise reprogramming astrocyte in a region in the nervous system, e.g., brain (e.g., striatum) or spinal cord, of a subject into a functional neuron.

As discussed above, the methods provided herein can comprise reprogramming a non-neuronal cell in a specific brain region (e.g., striatum) into a functional neuron. Exemplary brain regions that can be used in the methods provided herein can be in any of hindbrain, midbrain, or forebrain. In some embodiments, the methods provided herein comprise administering to a midbrain, striatum, or cortex of a subject a composition comprising a cell-programming agent that suppresses the expression or activity of PTB in a non-neuronal cell in mature retina or in the striatum, and allowing the non-neuronal cell to reprogram into the functional neuron. In some embodiments, the methods provided herein comprise administering to mature retina or in the striatum of a subject a composition comprising a cell-programming agent that suppresses the expression or activity of PTB/nPTB in a non-neuronal cell in the mature retina or in the striatum, and allowing the non-neuronal cell to reprogram into the functional neuron.

In some embodiments, the methods provided herein comprise reprogramming a non-neuronal cell into a functional neuron in a brain region, such as, but not limited to, medulla oblongata, medullary pyramids, olivary body, inferior olivary nucleus, rostral ventrolateral medulla, caudal ventrolateral medulla, solitary nucleus, respiratory center-respiratory groups, dorsal respiratory group, ventral respiratory group or apneustic centre, pre-bdtzinger complex, botzinger complex, retrotrapezoid nucleus, nucleus retrofacialis, nucleus retroambiguus, nucleus para-ambiguus, paramedian reticular nucleus, gigantocellular reticular nucleus, parafacial zone, cuneate nucleus, gracile nucleus, perihypoglossal nuclei, intercalated nucleus, prepositus nucleus, sublingual nucleus, area postrema, medullary cranial nerve nuclei, inferior salivatory nucleus, nucleus ambiguous, dorsal nucleus of vagus nerve, hypoglossal nucleus, metencephalon, pons, pontine nuclei, pontine cranial nerve nuclei, chief or pontine nucleus of the trigeminal nerve sensory nucleus, motor nucleus for the trigeminal nerve (v), abducens nucleus (vi), facial nerve nucleus (vii), vestibulocochlear nuclei (vestibular nuclei and cochlear nuclei) (viii), superior salivatory nucleus, pontine tegmentum, pontine micturition center (barrington's nucleus), locus coeruleus, pedunculopontine nucleus, laterodorsal tegmental nucleus, tegmental pontine reticular nucleus, parabrachial area, medial parabrachial nucleus, lateral parabrachial nucleus, subparabrachial nucleus (kdlliker-fuse nucleus), pontine respiratory group, superior olivary complex, medial superior olive, lateral superior olive, medial nucleus of the trapezoid body, paramedian pontine reticular formation, parvocellular reticular nucleus, caudal pontine reticular nucleus, cerebellar peduncles, superior cerebellar peduncle, middle cerebellar peduncle, inferior cerebellar peduncle, fourth ventricle, cerebellum, cerebellar vermis, cerebellar hemispheres, anterior lobe, posterior lobe, flocculonodular lobe, cerebellar nuclei, fastigial nucleus, interposed nucleus, globose nucleus, emboliform nucleus, dentate nucleus, midbrain (mesencephalon), tectum, corpora quadrigemina, inferior colliculi, superior colliculi, pretectum, tegmentum, periaqueductal gray, rostral interstitial nucleus of medial longitudinal fasciculus, midbrain reticular formation, dorsal raphe nucleus, red nucleus, ventral tegmental area, parabrachial pigmented nucleus, paranigral nucleus, rostromedial tegmental nucleus, caudal linear nucleus, rostral linear nucleus of the raphe, interfascicular nucleus, substantia nigra, pars compacta, pars reticulata, interpeduncular nucleus, cerebral peduncle, crus cerebri, mesencephalic cranial nerve nuclei, oculomotor nucleus (iii), edinger-westphal nucleus, trochlear nucleus (iv), mesencephalic duct (cerebral aqueduct, aqueduct of sylvius), forebrain (prosencephalon), diencephalon, epithalamus, pineal body, habenular nuclei, stria medullaris, taenia thalami, third ventricle, subcommissural organ, thalamus, anterior nuclear group, anteroventral nucleus (a.k.a. ventral anterior nucleus), anterodorsal nucleus, anteromedial nucleus, medial nuclear group, medial dorsal nucleus, midline nuclear group, paratenial nucleus, reuniens nucleus, rhomboidal nucleus, intralaminar nuclear group, centromedian nucleus, parafascicular nucleus, paracentral nucleus, central lateral nucleus, lateral nuclear group, lateral dorsal nucleus, lateral posterior nucleus, pulvinar, ventral nuclear group, ventral anterior nucleus, ventral lateral nucleus, ventral posterior nucleus, ventral posterior lateral nucleus, ventral posterior medial nucleus, metathalamus, medial geniculate body, lateral geniculate body, thalamic reticular nucleus, hypothalamus (limbic system) (hpa axis), anterior, medial area, parts of preoptic area, medial preoptic nucleus, suprachiasmatic nucleus, paraventricular nucleus, supraoptic nucleus (mainly), anterior hypothalamic nucleus, lateral area, parts of preoptic area, lateral preoptic nucleus, anterior part of lateral nucleus, part of supraoptic nucleus, other nuclei of preoptic area, median preoptic nucleus, periventricular preoptic nucleus, tuberal, medial area, dorsomedial hypothalamic nucleus, ventromedial nucleus, arcuate nucleus, lateral area, tuberal part of lateral nucleus, lateral tuberal nuclei, posterior, medial area, mammillary nuclei, posterior nucleus, lateral area, posterior part of lateral nucleus, optic chiasm, subfornical organ, periventricular nucleus, pituitary stalk, tuber cinereum, tuberal nucleus, tuberomammillary nucleus, tuberal region, mammillary bodies, mammillary nucleus, subthalamus, subthalamic nucleus, zona incerta, pituitary gland, neurohypophysis, pars intermedia (intermediate lobe), adenohypophysis, frontal lobe, parietal lobe, occipital lobe, temporal lobe, cerebellum, brainstem, centrum semiovale, corona radiata, internal capsule, external capsule, extreme capsule, subcortical, hippocampus, dentate gyrus, cornu ammonis (CA fields), cornu ammonis area 1 (CA1), cornu ammonis area 2 (CA2), cornu ammonis area 3 (CA3), cornu ammonis area 4 (CA4), amygdala, central nucleus of amygdala, medial nucleus of amygdala, cortical and basomedial nuclei of amygdala, lateral and basolateral nuclei of amygdala, extended amygdala, stria terminalis, bed nucleus of the stria terminalis, claustrum, basal ganglia, striatum, dorsal striatum, putamen, caudate nucleus, ventral striatum, nucleus accumbens, olfactory tubercle, globus pallidus, ventral pallidum, subthalamic nucleus, basal forebrain, anterior perforated substance, substantia innominata, nucleus basalis, diagonal band of broca, septal nuclei, medial septal nuclei, lamina terminalis, vascular organ of lamina terminalis, rhinencephalon (paleopallium), olfactory bulb, olfactory tract, anterior olfactory nucleus, piriform cortex, anterior commissure, uncus, periamygdaloid cortex, cerebral cortex, frontal lobe, cortex, primary motor cortex (precentral gyrus, MI), supplementary motor cortex, premotor cortex, prefrontal cortex, orbitofrontal cortex, dorsolateral prefrontal cortex, gyri, superior frontal gyrus, middle frontal gyrus, inferior frontal gyrus, Brodmann areas 4, 6, 8, 9, 10, 11, 12, 24, 25, 32, 33, 44, 45, 46, and 47, parietal lobe, cortex, primary somatosensory cortex (SI), secondary somatosensory cortex (S2), posterior parietal cortex, gyri, postcentral gyrus (primary somesthetic area), precuneus, Brodmann areas 1, 2, 3, 5, 7, 23, 26, 29, 31, 39, and 40, occipital lobe, cortex, primary visual cortex (VI), v2, v3, v4, v5/mt, gyri, lateral occipital gyrus, cuneus, Brodmann areas 17 (VI, primary visual cortex); 18, and 19, temporal lobe, cortex, primary auditory cortex (AI), secondary auditory cortex (A2), inferior temporal cortex, posterior inferior temporal cortex, gyri, superior temporal gyrus, middle temporal gyrus, inferior temporal gyrus, entorhinal cortex, perirhinal cortex, parahippocampal gyrus, fusiform gyrus, Brodmann areas 20, 21, 22, 27, 34, 35, 36, 37, 38, 41, and 42, medial superior temporal area (MST), insular cortex, cingulate cortex, anterior cingulate, posterior cingulate, retrosplenial cortex, indusium griseum, subgenual area 25, and Brodmann areas 23, 24; 26, 29, 30 (retrosplenial areas); 31, and 32.

In one aspect, the invention provides a method of generating a dopaminergic neuron in vivo. An exemplary method comprises administering to the striatum in the brain of a subject a composition comprising a cell-programming agent (e.g., a Cas effector protein and a coding sequence for gRNA against PTB and/or nPTB) that suppresses expression or activity of PTB and/or nPTB in a non-neuronal cell in the brain (e.g., a glial cell or astrocyte), and allowing the non-neuronal cell to reprogram into the dopaminergic neuron. In some embodiment, method comprises administering to the putamen of the subject a composition comprising a cell-programming agent (e.g., a Cas effector protein and a coding sequence for gRNA against PTB and/or nPTB) that suppresses expression or activity of PTB and/or nPTB in a non-neuronal cell in the brain (e.g., a glial cell or astrocyte), and allowing the non-neuronal cell to reprogram into the dopaminergic neuron. In some embodiment, method comprises administering exclusively to the putamen of the subject (e.g. avoiding administration to the caudate nucleus and/or other parts of the striatum) a composition comprising a cell-programming agent (e.g., a Cas effector protein and a coding sequence for gRNA against PTB and/or nPTB) that suppresses expression or activity of PTB and/or nPTB in a non-neuronal cell in the brain (e.g., a glial cell or astrocyte), and allowing the non-neuronal cell to reprogram into the dopaminergic neuron.

In another aspect, the invention provides a method of generating a RGC neuron in vivo. An exemplary method comprises administering to the mature retina of a subject a composition comprising a cell-programming agent (e.g., a Cas effector protein and a coding sequence for gRNA against PTB and/or nPTB) that suppresses expression or activity of PTB and/or nPTB in a non-neuronal cell in the mature retina (e.g., a glial cell or MG cell), and allowing the non-neuronal cell to reprogram into the RGC neuron.

In some embodiments, the methods provided herein comprise administering to a region in the nervous system, e.g., brain or spinal cord, of a subject a composition comprising a cell programming agent (e.g., a Cas effector protein and a coding sequence for gRNA against PTB and/or nPTB) that suppresses the expression or activity of PTB and/or nPTB in a non-neuronal cell in the region, and allowing the non-neuronal cell to reprogram into a functional neuron of a subtype that is predominant in the region.

Without being bound to a particular theory, the methods provided herein can take advantage of local induction signals in a region, e.g., a specific brain region, when reprogramming a non-neuronal cell into a functional neuron in vivo. For example, local signals in the striatum may induce the conversion of non-neuronal cells with PTB/nPTB suppressed into dopamine neurons. Local neurons, non-neuronal cells, e.g., astrocytes, microglia, or both, or other local constituents of the striatum can contribute to the subtype specification of the neuron that is generated from the non-neuronal cell under the induction of the cell-programming agent.

In some embodiments, the methods provided herein comprise administering to a brain region (e.g., striatum) of a subject a composition comprising a cell-programming agent (e.g., a Cas effector protein and a coding sequence for gRNA against PTB and/or nPTB) that suppresses the expression or activity of PTB/nPTB in a plurality of non-neuronal cell in the brain region, and the methods further comprise reprogramming at least about 5%, 10%, 20%, 25%, 30%, 35%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 99% of the non-neuronal cells to dopaminergic neurons.

In some embodiments, the methods provided herein comprise administering to the mature retina or a brain region (e.g., striatum) of a subject a composition comprising a cell-programming agent (e.g., a Cas effector protein and a coding sequence for gRNA against PTB and/or nPTB) that suppresses the expression or activity of PTB/nPTB in a plurality of non-neuronal cell in the brain region, and at least about 5%, 10%, 20%, 25%, 30%, 35%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 99% of the functional neurons generated by the methods are RGC or dopaminergic, respectively.

In some embodiments, the dopaminergic neuron generated in the methods provided herein expresses one or more markers of dopaminergic neurons, including, but not limited to, dopamine, tyrosine hydroxylase (TH), dopamine transporter (DAT), vesicular monoamine transporter 2 (VMAT2), engrailed homeobox 1 (En1), Nuclear receptor related-1 (Nurrl), G protein-regulated inward-rectifier potassium channel 2 (Girk2), forkhead box A2 (FoxA2), orthodenticle homeobox 2 (OTX2) and/or LIM homeobox transcription factor 1 alpha (Lmx1a).

In some embodiments, the dopamine neuron generated in the methods provided herein exhibit Ih current, which can be mediated by Hyperpolarization-activated cyclic nucleotide-gated (HCN) channels. Ih current can be characterized as a slowly activating, inward current, which can be activated by hyperpolarizing steps. For instance, under voltage clamp and the holding potential Vh is −40 mV, an inward slowly activating current can be triggered in a dopamine neuron, with a reversal potential close to −30 mV. The activation curve of Ih current characteristic of a dopamine neuron generated in the methods provided herein can range from −50 to −120 mV with a mid-activation point of −84-1 mV.

In some embodiments, the dopaminergic neurons generated in the methods provided herein have gene expression profile similar to a native dopaminergic neuron.

In some embodiments, the dopaminergic neurons generated in the methods provided herein release dopamine as a neurotransmitter.

A dopaminergic neuron generated in the methods provided herein can be of any subtype of dopaminergic neuron, including, but not limited to, A9 (e.g., immunopositive for Girk2), A10 (e.g., immunopositive for calbindin-D28 k), A11, A12, A13, A16, Aaq, and telencephalic dopamine neurons.

According to some embodiments of the present disclosure, the methods provided herein comprise reprogramming a non-neuronal cell in a region in the nervous system, e.g., mature retina or a region of the brain or spinal cord (e.g., striatum), of a subject to a functional neuron. In some embodiments, the functional neuron as discussed here is integrated into the neural network in the nervous system. As described herein, the reprogrammed functional neuron can form synaptic connections with local neurons, e.g., neurons that are adjacent to the reprogrammed functional neurons. For example, synaptic connections between the reprogrammed neuron and neighboring primary neuron (e.g., glutamatergic neurons), GABAergic interneurons, or other neighboring neurons (e.g., dopaminergic neuron, adrenergic neurons, or cholinergic neurons) can form as the reprogrammed neuron matures in vivo. Among these synaptic connections with local neurons, the reprogrammed functional neuron can be a presynaptic neuron, a postsynaptic neuron, or both.

In some embodiments, the reprogrammed functional neuron sends axonal projections to remote brain regions.

In some embodiments, a reprogrammed functional neuron can integrate itself into one or more existing neural pathways in the brain or spinal cord, for instance, but not limited to, superior longitudinal fasciculus, arcuate fasciculus, uncinate fasciculus, perforant pathway, thalamocortical radiations, corpus callosum, anterior commissure, amygdalofugal pathway, interthalamic adhesion, posterior commissure, habenular commissure, fornix, mammillotegmental fasciculus, incertohypothalamic pathway, cerebral peduncle, medial forebrain bundle, medial longitudinal fasciculus, myoclonic triangle, mesocortical pathway, mesolimbic pathway, nigrostriatal pathway, tuberoinfundibular pathway, extrapyramidal system, pyramidal tract, corticospinal tract or cerebrospinal fibers, lateral corticospinal tract, anterior corticospinal tract, corticopontine fibers, frontopontine fibers, temporopontine fibers, corticobulbar tract, corticomesencephalic tract, tectospinal tract, interstitiospinal tract, rubrospinal tract, rubro-olivary tract, olivocerebellar tract, olivospinal tract, vestibulospinal tract, lateral vestibulospinal tract, medial vestibulospinal tract, reticulospinal tract, lateral raphespinal tract, posterior column-medial lemniscus pathway, gracile fasciculus, cuneate fasciculus, medial lemniscus, spinothalamic tract, lateral spinothalamic tract, anterior spinothalamic tract, spinomesencephalic tract, spinocerebellar tract, spino-olivary tract, and spinoreticular tract.

Without being bound to a certain theory, local cellular environment can be correlated with the projections of a functional neuron generated according to some embodiments of the present disclosure. For instance, a functional neuron generated in striatum according to some embodiments of the methods provided herein can be affected by other cells in the local environment of striatum.

### Treatable Conditions/Diseases

In an aspect, the present disclosure provides a method of treating a neurological condition associated with degeneration of functional neurons in a region in the nervous system. An exemplary comprises administering to the region of the nervous system, e.g., mature retina or a region of the brain or spinal (e.g., striatum) or inner ear, of a subject in need thereof a composition comprising a cell-programming agent that suppresses the expression or activity of PTB/nPTB in a non-neuronal cell in the region, and allowing the non-neuronal cell to reprogram into a functional neuron (e.g., RGC or dopaminergic neuron), thereby replenishing the degenerated functional neurons in the region.

According to some embodiments of the present disclosure, methods provided herein comprise treating neurological conditions, including, but not limited to, Parkinson's disease, blindness, deafness, spinal cord injury, Alzheimer's disease, Huntington's disease, Schizophrenia, depression, and drug addiction.

Applicable neurological conditions can also include disorders associated with neuronal loss in spinal cord, such as, but not limited to, Amyotrophic lateral sclerosis (ALS) and motor neuron disease. The methods provided herein can also find use in treating or ameliorating one or more symptoms of neurodegenerative diseases including, but not limited to, autosomal dominant cerebellar ataxia, autosomal recessive spastic ataxia of Charlevoix-Saguenay, Corticobasal degeneration, Corticobasal syndrome, Creutzfeldt-Jakob disease, fragile X-associated tremor/ataxia syndrome, frontotemporal dementia and parkinsonism linked to chromosome 17, Kufor-Rakeb syndrome, Lyme disease, Machado-Joseph disease, Niemann-Pick disease, pontocerebellar hypoplasia, Refsum disease, pyruvate dehydrogenase complex deficiency, Sandhoff disease, Shy-Drager syndrome, Tay-Sachs disease, and Wobbly hedgehog syndrome.

As provided herein, “neurodegeneration” or its grammatical equivalents, can refer to the progressive loss of structure, function, or both of neurons, including death of neuron.

Neurodegeneration can be due to any type of mechanisms. A neurological condition the methods provided herein are applicable to can be of any etiology. A neurological condition can be inherited or sporadic, can be due to genetic mutations, protein misfolding, oxidative stress, or environment exposures (e.g., toxins or drugs of abuse).

In some embodiments, the methods provided herein treat a neurological condition associated with degeneration of dopaminergic neurons in a brain region. In some embodiments, the methods provided herein treat a neurological condition associated with degeneration of RGC neurons in the mature retina. In other embodiments, the methods provided herein treat a neurological condition associated with degeneration of any type of neurons, such as, but not limited to, glutamatergic neurons, GABAergic neurons, cholinergic neurons, adrenergic neurons, dopaminergic neurons, or any other appropriate type neurons that release neurotransmitter aspartate, D-serine, glycine, nitric oxide (NO), carbon monoxide (CO), hydrogen sulfide (H2S), norepinephrine (also known as noradrenaline), histamine, serotonin, phenethylamine, N-methylphenethylamine, tyramine, 3-iodothyronamine, octopamine, tryptamine, somatostatin, substance P, opioid peptides, adenosine triphosphate (ATP), adenosine, or anandamide. The methods provided herein can find use in treating a neurological condition associated with neuronal degeneration in any region, such as, but limited to, midbrain regions (e.g., substantial nigra or ventral tegmental area), forebrain regions, hindbrain regions, or spinal cord. The methods provided herein can comprise reprogramming non-neuronal cells to functional neurons in any appropriate region (s) in the nervous system in order to treat a neurological condition associated with neuronal degeneration.

Methods provided herein can find use in treating or ameliorating one or more symptoms associate with Parkinson's disease. Parkinson's disease is a neuro-degenerative disease with early prominent functional impairment or death of dopaminergic neurons in the substantia nigra pars compacta (SNpc). The resultant dopamine deficiency within the basal ganglia can lead to a movement disorder characterized by classical parkinsonian motor symptoms. Parkinson's disease can also be associated with numerous non-motor symptoms. One standard for diagnosis of Parkinson's disease can be the presence of SNpc degeneration and Lewy pathology at post-mortem pathological examination. Lewy pathology can include abnormal aggregates of asynuclein protein, called Lewy bodies and Lewy neurites. Patients with Parkinson's disease can exhibit a number of symptoms, including motor symptoms and non-motor symptoms. Methods provided herein can treat or ameliorate one or more of these motor or non-motor symptoms associated with Parkinson's disease. Motor symptoms of Parkinson's disease (Parkinsonism symptoms) can include bradykinesia (slowness), stiffness, impaired balance, shuffling gait, and postural instability. Motor features in patients with Parkinson's disease can be heterogeneous, which has prompted attempts to classify subtypes of the disease, for instance, tremor-dominant Parkinson's disease (with a relative absence of other motor symptoms), non-tremor-dominant Parkinson's disease (which can include phenotypes described as akinetic-rigid syndrome and postural instability gait disorder), and an additional subgroup with a mixed or indeterminate phenotype with several motor symptoms of comparable severity. Non motor symptoms of Parkinson's disease can include olfactory dysfunction, cognitive impairment, psychiatric symptoms (e.g., depression), sleep disorders, autonomic dysfunction, pain, and fatigue. These symptoms can be common in early Parkinson's disease. Non-motor features can also be frequently present in Parkinson's disease before the onset of the classical motor symptoms. This premotor or prodromal phase of the disease can be characterized by impaired olfaction, constipation, depression, excessive daytime sleepiness, and rapid eye movement sleep behaviour disorder.

In some embodiments, methods provided herein mitigate or slow the progression of Parkinson's disease. Progression of Parkinson's disease can be characterized by worsening of motor features. As the disease advances, there can be an emergence of complications related to long-term symptomatic treatment, including motor and non-motor fluctuations, dyskinesia, and psychosis.

One pathological feature of Parkinson's disease can be loss of dopaminergic neurons within the substantial nigra, e.g., substantial nigra pars compacta (SNpc). According to some embodiments, methods provided herein replenish dopamine (secreted from converted dopamine neuron in the striatum) diminished due to loss of dopamine neuron in substantial nigra (e.g., SNpc) of a patient. Neuronal loss in Parkinson's disease can also occur in many other brain regions, including the locus ceruleus, nucleus basalis of Meynert, pedunculopontine nucleus, raphe nucleus, dorsal motor nucleus of the vagus, amygdala, and hypothalamus. In some embodiments, methods of treating or ameliorating one or more symptoms of Parkinson's disease in a subject as provided herein include reprogramming non-neuronal cells to functional neurons in brain regions experiencing neuronal loss in a patient with Parkinson's disease.

Methods provided herein can find use in treating Parkinson's disease of different etiology. For example, there can be Parkinson's disease as a result of one or more genetic mutations, such as, but not limited to, mutations in genes SNCA, LRRK2, VPS35, ElF4G1, DNAJC13, CHCHD2, Parkin, PINK1, DJ-1, ATP13A2, C9ORF72, FBX07, PLA2G6, POLG1, SCA2, SCA3, SYNJ1, RAB39B, and possibly one or more genes affected in 22qII.2 microdeletion syndrome. Or there can be Parkinson's disease with no known genetic traits.

As provided herein, the one or more symptoms of Parkinson's disease the methods provided herein can ameliorate can include not only the motor symptoms and non-symptoms as described above, but also pathological features at other levels. For example, reduction in dopamine signaling in the brain of a patient with Parkinson's disease can be reversed or mitigated by methods provided herein by replenishing functional dopamine neurons, which can be integrated into the neural circuitry and reconstruct the dopamine neuron projections to appropriate brain regions.

In an aspect, the present disclosure also provides methods of restoring dopamine release in subject with a decreased amount of dopamine biogenesis compared to a normal level. An exemplary method comprises reprogramming a non-neuronal cell in a brain region of the subject (e.g., striatum), and allowing the non-neuronal cell to reprogram into a dopaminergic neuron, thereby restoring at least 50% of the decreased amount of dopamine. In some embodiments, the reprogramming is performed by administering to the brain region of the subject (e.g., striatum) a composition comprising a cell-programming agent that suppresses the expression or activity of PTB/nPTB in a non-neuronal cell (e.g., an astrocyte) in the brain region. In some embodiments, the methods provided herein restore at least about 20%, 25%, 30%, 35%, 40%, 45%, 50%, 55%, 60%, 65%, 70%, 75%, 80%, 85%, 90%, 95%, or 98% of the decreased amount of dopamine. In some embodiments, the methods provided herein restore about 20%, 25%, 30%, 35%, 40%, 45%, 50%, 55%, 60%, 65%, 70%, 75%, 80%, 85%, 90%, 95%, 98%, or 100% of the decreased amount of dopamine. In some embodiments, the methods provided herein restore at least about 20%, 25%, 30%, 35%, 40%, 45%, 50%, 55%, 60%, 65%, 70%, 75%, 80%, 85%, 90%, 95%, or 98% of the decreased amount of dopamine. In some embodiments, the methods provided herein restore at least about 50% of the decreased amount of dopamine.

### Pharmaceutical Composition

In one aspect, the present disclosure provides pharmaceutical compositions comprising a cell-programming agent in an amount effective to reprogram a mammalian non-neuronal cell to a mature neuron by suppressing the expression or activity of PTB/nPTB in the non-neuronal cell. An exemplary pharmaceutical composition can further comprise a pharmaceutically acceptable carrier or excipient. As described above, a cell-programming agent as provided herein can be a Cas effector protein and a coding sequence for a gRNA against PTB/nPTB.

A pharmaceutical composition provided herein can include one or more carriers and excipients (including but not limited to buffers, carbohydrates, mannitol, proteins, peptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In another instance, the composition is substantially free of preservatives. In other embodiments, the composition contains at least one preservative. General methodology on pharmaceutical dosage forms can be found in Ansel et ah, Pharmaceutical Dosage Forms and Drug Delivery Systems (Lippencott Williams & Wilkins, Baltimore Md. (1999)). It will be recognized that, while any suitable carrier known to those of ordinary skill in the art can be employed to administer the pharmaceutical compositions described herein, the type of carrier can vary depending on the mode of administration. Suitable formulations and additional carriers are described in Remington “The Science and Practice of Pharmacy” (20th Ed., Lippincott Williams & Wilkins, Baltimore Md.), the teachings of which are incorporated by reference in their entirety herein.

An exemplary pharmaceutical composition can be formulated for injection, inhalation, parenteral administration, intravenous administration, subcutaneous administration, intramuscular administration, intradermal administration, topical administration, or oral administration.

In certain embodiments, the pharmaceutical composition comprising an AAV vector encoding a Cas effector and a coding sequence for a gRNA against PTB/nPTB can be injected into the mature retina, or the striatum of a subject's brain.

As one of ordinary skills in the art will appreciate, pharmaceutical compositions can comprise any appropriate carrier or excipient, depending on the type of cell-programming agent and the administration route the composition is designed for. For example, a composition comprising a cell-programming agent as provided herein can be formulated for parenteral administration and can be presented in unit dose form in ampoules, pre-filled syringes, small volume infusion or in multi dose containers with an added preservative. The composition can take such forms as suspensions, solutions, or emulsions in oily or aqueous vehicles, for example solutions in aqueous polyethylene glycol. For example, for injectable formulations, a vehicle can be chosen from those known in the art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles.

The formulation can also comprise polymer compositions which are biocompatible, biodegradable, such as poly (lactic-co-glycolic) acid. These materials can be made into micro or nanospheres, loaded with drug and further coated or derivatized to provide superior sustained release performance.

Vehicles suitable for periocular or intraocular injection include, for example, suspensions of active agent in injection grade water, liposomes, and vehicles suitable for lipophilic substances and those known in the art. A composition as provided herein can further comprise additional agent besides a cell-programming agent and a pharmaceutically acceptable carrier or excipient. For example, additional agent can be provided for promoting neuronal survival purpose. Alternatively or additionally, additional agent can be provided for monitoring pharmacodynamics purpose. In some embodiments, a composition comprises additional agent as a penetration enhancer or for sustained release or controlled release of the active ingredient, e.g., cell-programming agent.

A composition provided herein can be administered to a subject in a dosage volume of about 0.0005, 0.001, 0.002, 0.005, 0.01, 0.02, 0.05, 0.1, 0.15, 0.2, 0.25, 0.3, 0.35, 0.4, 0.45, 0.5, 0.55, 0.6, 0.7, 0.8, 0.9, 1.0 mL, or more. The composition can be administered as a 1, 2, 3, 4, 5, 6, 7, 8, 9, 10, or more dose-course regimen. Sometimes, the composition can be administered as a 2, 3, or 4 dose-course regimen. Sometimes the composition can be administered as a 1 dose-course regimen.

The administration of the first dose (e.g., an AAV vector encoding a Cas effector and a gRNA against PTB) and second dose (e.g., an AAV vector encoding a Cas effector and a gRNA against nPTB) of the 2 dose-course regimen can be separated by about 0 day, 1 day, 2 days, 5 days, 7 days, 14 days, 21 days, 30 days, 2 months, 4 months, 6 months, 9 months, 1 year, 1.5 years, 2 years, 3 years, 4 years, 5 years, 10 years, 20 years, or more. A composition described herein can be administered to a subject once a day, once a week, once two weeks, once a month, a year, twice a year, three times a year, every 1, 2, 3, 4, 5, 6, 7, 8, 9, 10, or more years.

Sometimes, the composition can be administered to a subject every 2, 3, 4, 5, 6, 7, or more years. Sometimes, the composition can be administered to a subject once.

### Further Aspects

Some embodiments of the disclosure provide methods and compositions for cell or tissue transplantation. An exemplary method can comprise reprogramming a non-neuronal cell to a neuron in vitro, and transplanting the reprogrammed neuron into a brain region in a subject. In some embodiments, in vitro reprogramming can be performed according to the methods provided herein. An exemplary composition can comprise a neuron reprogrammed according to any embodiment of the methods provided herein.

In other embodiments, a method provided herein comprises reprogramming a non-neuronal cell to a neuron in vivo, and explanting the reprogrammed neuron. In some embodiments, the explant comprises a brain tissue comprising the reprogramed neuron. In some embodiments, the explant is transplanted into a brain region of a subject. As provided herein, the transplantation of neurons reprogrammed according to the methods provided herein can be used to replenish degenerated neurons in a subject suffering a condition associated with neuronal loss.

Some other aspects of the present disclosure relate to an animal that comprise neurons reprogrammed according to any embodiment of the methods provided herein.

As provided herein, an animal can be any mammal. An animal can be a human. An animal can be a non-human primate, such as, but not limited, rhesus macaques, crab-eating macaques, stump-tailed macaques, pig-tailed macaques, squirrel monkeys, owl monkeys, baboons, chimpanzees, marmosets and spider monkeys. An animal can be a research animal, a genetically modified animal, or any other appropriate type of animal.

Also provided herein is a brain tissue (e.g., explant) of an animal that comprises one or more neurons reprogrammed according to any embodiment of the present disclosure. Such brain tissue can be live. In some embodiments, a brain tissue can be fixed by any appropriate fixative. A brain tissue can be used for transplantation, medical research, basic research, or any type of purposes.

The disclosure demonstrates that the method is applicable to disease models of neurodegeneration. For example, the disclosure shows that astrocyte-to-neuron conversion strategy can work in a chemical-induced Parkinson's disease model. The methods and compositions can convert astrocytes to neurons including dopaminergic, glutamatergic and GABAergic neurons, these neurons are able to form synapses in the brain, and remarkably, the converted neurons can efficiently reconstruct the lesioned nigrostriatal pathway to correct measurable Parkinson's phenotypes. The effectiveness of this method was demonstrated both in astrocytes in culture (human and mouse) as well as in vivo in a mouse Parkinson's disease model. Therefore, this strategy has the potential to cure Parkinson's disease, which can also be applied to a wide range of neurodegenerative diseases (e.g., other neurological diseases associated with neuronal dysfunction).

In some embodiments, the approach of the disclosure exploits the genetic foundation of a neuronal maturation program already present, but latent, in both mammalian astrocytes that progressively produce mature neurons once they are reprogrammed by PTB suppression. These findings provide a clinically feasible approach to generate neurons from local astrocytes in mammalian brain using a single dose of a vector comprising coding sequence for a Cas effector and a gRNA against PTB/nPTB. The phenotypes of PTB/nPTB knockdown-induced neurons can be a function of the context in which they are produced and/or the astrocytes from which they are derived.

The disclosure demonstrates the potent conversion of astrocytes to neurons (e.g., dopamine neurons in the striatum). More particularly, the disclosure shows that in a primate model, the strategy efficiently can convert astrocytes to neurons, thus satisfying all five factors for in vivo reprogramming. The data provided herein show that PTB reduction in the primate brain can convert astrocytes to dopamine neurons (e.g., dopaminergic neurons).

A “therapeutically effective amount” of a composition of the disclosure will vary according to factors such as the disease state, age, sex, and weight of the individual, and the ability of the composition to elicit a desired response in the individual. A therapeutically effective amount can also be one in which any toxic or detrimental effects of the composition are outweighed by the therapeutically beneficial effects. Without wishing to be bound by a particular theory, it is contemplated that, in some cases, a therapeutically effective amount of cell-programming agent as provided herein can be an amount of cell-programming agent that converts a certain proportion of astrocytes in a brain region that experiences neuronal loss, conversion of such proportion of astrocytes to functional neurons in the brain region is sufficient to ameliorate or treating the disease or condition associated with the neuronal loss in the brain region, and meanwhile, such proportion of astrocytes does not exceed a threshold level that can lead to aversive effects that can overweigh the beneficial effects brought by the neuronal conversion, for instance, due to excessive reduction in the number of astrocytes in the brain region as a direct consequence of the neuronal conversion.

The following examples are intended to illustrate but not limit the disclosure. While they are typical of those that might be used, other procedures known to those skilled in the art may alternatively be used.

### EXAMPLES

### Example 1

### Methods and Materials

### Mice and Cell Lines

C57BL/6 mice were purchased from Shanghai SLAC Laboratory. The mice were housed in a light/dark cycle room with water and food. All animal experiments were performed and approved by the Animal Care and Use Committee of the CEBSIT, Chinese Academy of Sciences, Shanghai, China. The Cos7, 293T and N2a cell lines were obtained from Cell bank of Shanghai Institute of Biochemistry and Cell Biology (SIBCB), Chinese Academy of Sciences (CAS), and cultured in DMEM with 10% FBS and 1% penicillin/streptomycin in a 37° C. incubator under 5% CO2.

Transfection, qPCR and RNA-Seq

Transient transfection was conducted with 4 μg vectors expressing CAG-CasRx-P2A-GFP+2 μg U6-gRNA-CMV-mCherry plasmid using Lipofectamine 3000. CAG-CasRx-P2A-GFP plasmid was used as a control. Two days after transient transfection, around 30K GFP and mCherry double-positive (GFP top 20%) cells were collected by fluorescence activated cell sorting (FACS) and lysed for qPCR analysis. RNA was first extracted using Trizol (Ambion) and then converted to cDNA using a reverse transcription kit (HiScript Q RT SuperMix for qPCR, Vazyme, Biotech). The amplification was tracked by AceQ qPCR SYBR Green Master Mix (Vazyme, Biotech). For the RNA-seq, Cos7 cells were cultured in 10-cm dishes. Around 100 k positive cells (GFP top 20%) were isolated by FACS, RNA was extracted and reverse transcribed to cDNA, which was then used for RNA-seq. Analysis of the RNA-seq data was performed as previously reported (Zhou et al., 2018, Cell 181: 590-603) and presented as the mean of all repeats.

### Guide RNA Sequences

The guide sequences (i.e. the sequence that is complementary to the PTB mRNA sequence to be targeted) in the guide RNAs as used are shown in Table 1.

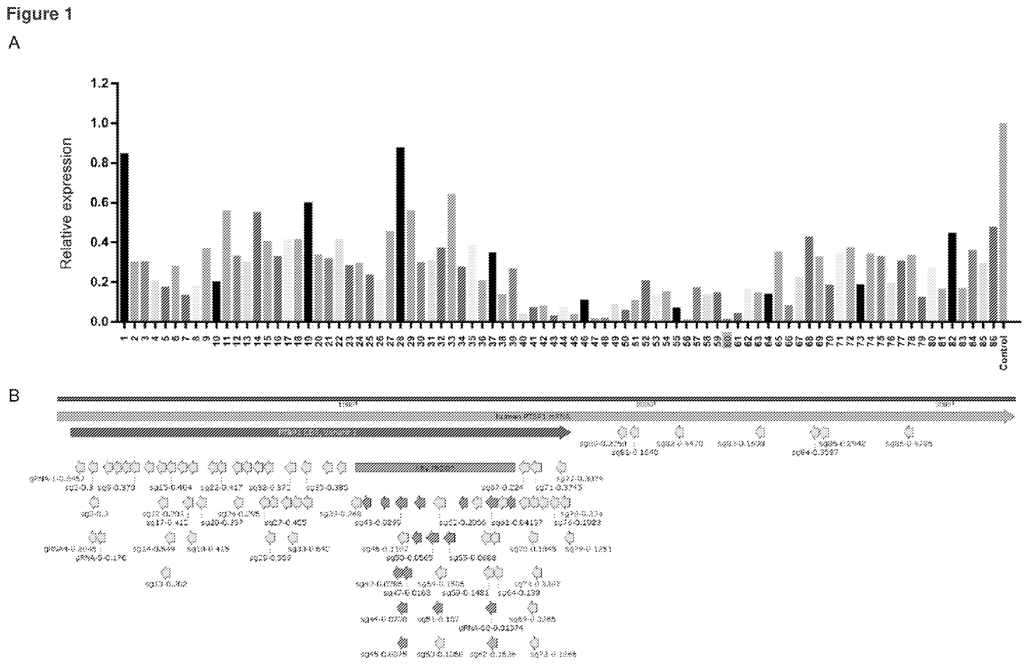

TABLE 1
Guide sequences of guide RNAs and the relative expression level (vs control
without gRNA) achieved by the respective guide RNAs. High efficiency gRNAs, all
located in the key region (positions 951-1487 of SEQ ID NO: 87, which is SEQ ID
NO: 88; see also FIG. 1), are indicated in bold print.

| guide | | | SEQ ID |
|---|---|---|---|
| RNA | Sequence | Efficiency | No |
| gRNA56 | cagcugcacguucuggugcuucgag | 0.0109 | 56 |
| gRNA60 | uucuggaaguucuuggagcccggcuucuug | 0.0137 | 60 |
| gRNA47 | aggcuuuggggugugacucucucuggguug | 0.0164 | 47 |
| gRNA48 | cugcacgucaccguagacgccgaaaagaau | 0.0183 | 48 |
| gRNA43 | acucucucuggguugagguugcugaccagcaauacag | 0.0299 | 43 |
| gRNA45 | acucucucuggguugagguugcugaccagc | 0.0376 | 45 |
| gRNA40 | ccagggcgccguggacguucggaacgg | 0.0378 | 40 |
| gRNA61 | aauauguucuggaaguucuuggagcccggcuucuug | 0.0414 | 61 |
| gRNA50 | ccguccgccaucugcacuagggcguucuccuucuuauug | 0.0565 | 50 |
| gRNA55 | cuucccgugcagcuugugcccguucagguggcuc | 0.0689 | 55 |
| gRNA41 | ggauggcgauccgaccugccgccgc | 0.0721 | 41 |
| gRNA44 | cucucucuggguugagguugcugaccagca | 0.0738 | 44 |
| gRNA42 | gguugcugaccagcaauacagaauuuccug | 0.0785 | 42 |
| gRNA66 | agauccuccucggagacugagggggg | 0.0804 | 66 |
| gRNA49 | aucuucacgcgcugcacgucaccguagacgccg | 0.0873 | 49 |
| gRNA51 | gcugggccugguugccguccgccaucugca | 0.1074 | 51 |
| gRNA46 | ugugacucucucuggguugagguugcugaccagc | 0.1107 | 46 |
| gRNA79 | ccugugccccuagaugguggacuuggag | 0.1251 | 79 |
| gRNA07 | acgccugcacuucggcugucaccuuug | 0.1346 | 07 |
| gRNA58 | gcccggcuucuugaagcggugcagggguga | 0.1354 | 58 |
| gRNA53 | uggccagcugggccugguugccguccgcca | 0.1358 | 53 |
| gRNA38 | agaggcugagauuauaccaggugcaccg | 0.1365 | 38 |
| gRNA64 | cguggccgagggcgggaauauguucugg | 0.1390 | 64 |
| gRNA63 | gggcgggaauauguucuggaaguucuugga | 0.1481 | 63 |
| gRNA59 | guucuuggagcccggcuucuugaagcggug | 0.1481 | 59 |
| gRNA54 | uggcucauggccagcugggccugguugccguccgcc | 0.1506 | 54 |
| gRNA62 | uauguucuggaaguucuuggagcccggcuu | 0.1636 | 62 |
| gRNA81 | ccgggaacucccgcugccacagucacc | 0.1640 | 81 |
| gRNA83 | uauugcuaggcacagacgucugcaacugc | 0.1698 | 83 |
| gRNA57 | guugccguaguccuuggucaggcccugguc | 0.1713 | 57 |
| gRNA05 | cagaagccgaguugcugcucaugau | 0.1760 | 05 |
| gRNA08 | agcuuccggauguggaucacucuag | 0.1822 | 08 |
| gRNA70 | gugccaucuugcgguccuucuggaagaacu | 0.1845 | 70 |
| gRNA73 | aucagugccaucuugcgguccuucugg | 0.1866 | 73 |
| gRNA76 | uucuccccgaggucgugguugugcagguc | 0.1923 | 76 |
| gRNA10 | cuucagcaucaggagguuggugaccuuccc | 0.2012 | 10 |
| gRNA04 | cgguccguuagugacacaaguagag | 0.2045 | 04 |
| gRNA36 | uuguuguacuugacguugaggcuggugagc | 0.2064 | 36 |
| gRNA52 | gcucauggccagcugggccugguugccguccgccaucugc | 0.2066 | 52 |
| gRNA26 | augaucuucaacacugugccgaacuuggag | 0.2073 | 26 |
| gRNA67 | cuugaauccuuugacgacgcccccauugcugg | 0.2240 | 67 |
| gRNA25 | ugcagcacauccagggucacaggguag | 0.2354 | 25 |
| gRNA39 | ccugcagcuugaggaauggcaaaggugggaggg | 0.2679 | 39 |
| gRNA34 | agucgaugcgcagcgugcagcaggcguugu | 0.2758 | 34 |
| gRNA80 | ucccugucaccgcagggugagcaaggug | 0.2758 | 80 |
| gRNA06 | cuucuugcugucauuuccguuugcugcag | 0.2813 | 06 |
| gRNA23 | gguucuccacgaugauccugagcacggggc | 0.2832 | 23 |
| gRNA85 | acccacugggcggccccgccugcucucugg | 0.2942 | 85 |
| gRNA24 | gaagagguucuccacgaugauccugagcac | 0.2952 | 24 |
| gRNA30 | guccgcauacugcagcagggccugg | 0.2980 | 30 |
| gRNA02 | uaaacgguccguuagugacacaaguagaga | 0.2990 | 02 |
| gRNA13 | ccaugguguuggcagccuccuccguguuc | 0.3018 | 13 |
| gRNA03 | ugauaaacgguccguuagugacacaaguag | 0.3025 | 03 |
| gRNA77 | gaaggagacccgcagguggugguucucccc | 0.3074 | 77 |
| gRNA31 | uguucugcccguccagcgacagcuuggcgu | 0.3110 | 31 |
| gRNA21 | ggccagguuccccgacuggaccgaguucac | 0.3161 | 21 |
| gRNA69 | ccaucuugcgguccuucuggaagaacuug | 0.3265 | 69 |
| gRNA16 | cuggauguagaugggcuggccgcgcagcac | 0.3280 | 16 |
| gRNA75 | ccuggaccgccuccuccacggagcccaucugg | 0.3310 | 75 |
| gRNA12 | uuggcagccuccuccguguucaucucgaug | 0.3322 | 12 |
| gRNA78 | ugguggacuuggagaaggagacccgcaggu | 0.3341 | 78 |
| gRNA20 | gggccgccugggcccgcgccugguugggag | 0.3368 | 20 |
| gRNA74 | gcccaucuggaucagugccaucuugcgguc | 0.3388 | 74 |
| gRNA71 | cccaucuggaucagugccaucuugcgguccuucugg | 0.3475 | 71 |
| gRNA37 | ucugguccagcgagggcuggcuguccccgg | 0.3479 | 37 |
| gRNA65 | auguuggagaggugcagcguggccgagggggg | 0.3523 | 65 |
| gRNA84 | cuggguccugccugugacuuaauuac | 0.3597 | 84 |
| gRNA09 | agaugacuucccccuccgugacgucgaugggg | 0.3703 | 09 |
| gRNA32 | cagcaggcguuguagauguucugcccguccagcgac | 0.3716 | 32 |
| gRNA72 | aucagugccaucuugcgguccuucuggaag | 0.3741 | 72 |
| gRNA35 | uuguacuugacguugaggcuggugagcuugg | 0.3847 | 35 |
| gRNA15 | ccgagguguaguaguucaccaugguguuggc | 0.4038 | 15 |
| gRNA17 | gcuccuugugguuggagaacuggauguaga | 0.4123 | 17 |
| gRNA18 | cugucggucuucagcuccuugugguuggag | 0.4147 | 18 |
| gRNA22 | cguccacggccgccgccgaggcagcc | 0.4147 | 22 |
| gRNA68 | cuugaauccuuugacgacgcccccauugcu | 0.4288 | 68 |
| gRNA82 | acaccccggguggguguguggcugcagg | 0.4470 | 82 |
| gRNA27 | cugguuguucuuggugaaggugaugaucuu | 0.4553 | 27 |
| gRNA86 | cccccgggacaggacgcgggggcgguag | 0.4785 | 86 |
| gRNA14 | guguaguaguucaccaugguguuggcagc | 0.5490 | 14 |
| gRNA11 | gccuccuccguguucaucucgaugaaggcc | 0.5593 | 11 |
| gRNA29 | cugcagcagggccuggaacugguuguucuu | 0.5593 | 29 |
| gRNA19 | gagcugucggucuucagcuccuugugguugg | 0.5980 | 19 |
| gRNA33 | gcgugcagcaggcguuguagauguucugcc | 0.6425 | 33 |
| gRNA01 | uccccgcuuuguaccaacggcuauaucuggg | 0.8458 | 01 |
| gRNA28 | gcagggccuggaacugguuguucuugguga | 0.8756 | 28 |
| Control | no gRNA | 0.9965 | |

### Stereotactic Injection and Immunofluorescence Staining

AAV8 was used in this study. Stereotactic injections in the mice were performed as previously described (Zhou et al., 2014, Elife 3, e02536, doi:10.7554/eLife.02536). The mice were placed in a stereotactic frame. Next, the skin over the skull was shaven and opened using a razor. A craniotomy with coordinates (AP+0.8 mm, ML±1.6 mm) was made over the boundary of frontal and parietal bones, allowing the placement of an injection micropipette (˜20 μm outside diameter at the tip). The viral solution containing either AAV-GFAP-CasRx-Ptbp/+AAV-GFAP-mCherry or AAV-GFAP-CasRx+AAV-GFAP-mCherry was injected slowly (˜0.3 μl/min). Mice were injected in the striatum (AP+0.8 mm, ML±1.6 mm and DV 2.6 mm) with AAVs (>1×10¹² vg/ml, 1 μL, mice aged 8-10 weeks). For Macaca fascicularis, the immunosuppressant dexamethasone (1.5 mg/kg) was administrated via intramuscular injection, around 14 hours before AAV injection. For AAV injection, both sides of the putamen were injected with 80 μL (>1×10¹² vg/ml) AAV-GFAP-CasRx-Ptbp/+AAV-GFAP-mCherry and AAV-GFAP-CasRx+AAV-GFAP-mCherry, respectively. The volume ratio between GFAP-mCherry and GFAP-CasRx or GFAP-CasRx-Ptbp1 were 1:20. For immunofluorescence staining, the brains were perfused and fixed with 4% paraformaldehyde (PFA) overnight, and kept in 30% sucrose for at least 12 hours for mice and 2 weeks for Macaca fascicularis. Brains were sectioned after embedding and freezing, and slices with the thickness of 30 μm for mice and 30 μm for Macaca fascicularis were used for immunofluorescence staining. Brain sections were rinsed thoroughly with 0.1 M phosphate buffer (PB). Primary antibodies: Rabbit-anti-NeuN (1:500, 24307S, Cell Signaling Technology), Guinea Pig anti-NeuN antibody (1:500, ABN90, Millipore), Mouse anti-Flag (1:2000, F3165, Sigma), Rabbit anti-TH anti-body (1:500, AB152, Millipore), Rat anti-DAT (1:100, MAB369, Millipore) and Rabbit anti-RBPMS (Proteintech, Cat #15187-1-AP). Secondary antibodies: Alexa Fluora 488 AffiniPure Donkey Anti-Rabbit IgG (H+L) (1:500, 711-545-152, Jackson ImmunoResearch) Alexa Fluora 488 AffiniPure Donkey Anti-Mouse IgG (H+L) (1:500, 715-545-150, Jackson ImmunoResearch) Cy5-AffiniPure Donkey Anti-Guinea Pig IgG (H+L) (1:500, 706-175-148, Jackson ImmunoResearch) Cy5 AffiniPure Donkey Anti-Rabbit IgG (H+L) (1:500, 711-175-152, Jackson ImmunoResearch) were used in this study. After antibody incubation, slices were washed and then covered with mountant (Life Technology). Images were collected under an Olympus FV3000 microscope.

### Subretinal Injection and Immunofluorescence Staining

NMDA and AAVs (AAV8) were introduced via subretinal injection. For subretinal injection, mice were aneathetized and AAVs were slowly injected into the subretinal space. To determine the conversion in intact retinas, in total 1 μl AAV-GFAP-GFP-Cre (0.2 μl) and AAV-GFAP-CasRx-Ptbp1 (0.4 μl), or AAV-GFAP-GFP-Cre (0.2 μl) and AAV-GFAP-CasRx (0.4 ml) and PBS (0.4 μl) or AAV-GFAP-GFP-Cre (0.2 μl) and AAV-GFAP-CasRx-Ptbp1 (0.4 μl) and AAV-GFAP-β-catenin (0.4 μl) were delivered to the retina via subretinal injection (Ai9 and C57BL/6 mice, aged 4 months). Two-three weeks after AAV injection, the eyes and optic nerves were collected and fixed with 4% paraformaldehyde (PFA), and then maintained in the solution containing 30% sucrose. After embedding, the eyes were sectioned and slices were washed and covered with mountant (Life Technology). The images were collected using Olympus FV3000 microscope.

### Results Example 1

In this study, we designed a system that could specifically downregulate Ptbp1 expression in both mice, Macaca fascicularis and human cells, and further showed that CasRx-mediated downregulation of Ptbp1 could directly convert astrocytes into dopamine neurons with high efficiency in the striatum of Macaca fascicularis. Taken together, our study provides the pre-clinical evidence that Ptbp1-mediated dopamine neuron conversion is also feasible in primates, i.e. in both human and non-human primates, paving the road for the clinical trials.

To knock down the expression of Ptbp1, we used the RNA-guided and RNA-targeting CRISPR protein CasRx, which is an efficient and specific approach for downregulation of RNAs. To examine the efficiency of CasRx-mediated knockdown of Ptbp1, we designed 86 gRNAs targeting the human Ptbp1 gene. We observed that co-transfection of a vector containing CasRx gene with gRNAs that target Ptbp1, resulted in reduction of Ptbp1 mRNA in human 293T cells, 2 days after transfection (FIG. 1A; for gRNA sequences, see Table 1). Specifically, targeting of a key region in human Ptbp1 gene (positions 951-1487 of SEQ ID NO: 87, which correspond to position 1-536 of SEQ ID NO: 88) frequently induced potent downregulation (FIG. 1B). In this experiment, gRNAs targeting this key region achieved a knock-down to 40% or lower, and potent knock-down to 10% or lower is only observed in this key region.

| SEQ ID NO: 88: | |
|---|---|
| gccattcctc aagctgcagg cctttccgtt ccgaacgtcc acggcgccct ggcccccctg | 60 |
| gccatcccct cggcggcggc ggcagctgcg gcggcaggtc ggatcgccat cccgggcctg | 120 |
| gcgggggcag gaaattctgt attgctggtc agcaacctca acccagagag agtcacaccc | 180 |
| caaagcctct ttattctttt cggcgtctac ggtgacgtgc agcgcgtgaa gatcctgttc | 240 |
| aataagaagg agaacgccct agtgcagatg gcggacggca accaggccca gctggccatg | 300 |
| agccacctga acgggcacaa gctgcacggg aagcccatcc gcatcacgct ctcgaagcac | 360 |
| cagaacgtgc agctgccccg cgagggccag gaggaccagg gcctgaccaa ggactacggc | 420 |
| aactcacccc tgcaccgctt caagaagccg ggctccaaga acttccagaa catattcccg | 480 |
| ccctcggcca cgctgcacct ctccaacatc ccgccctcag tctccgagga ggatct | 536 |

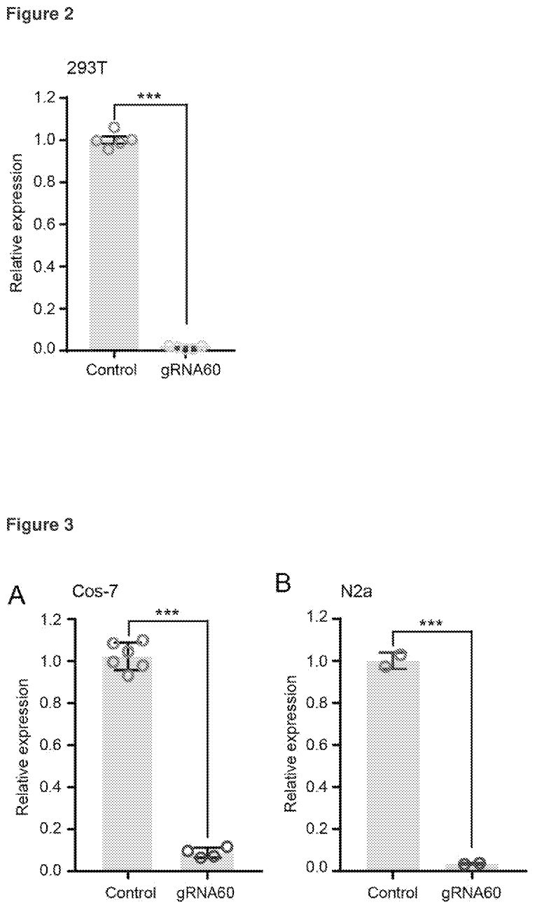

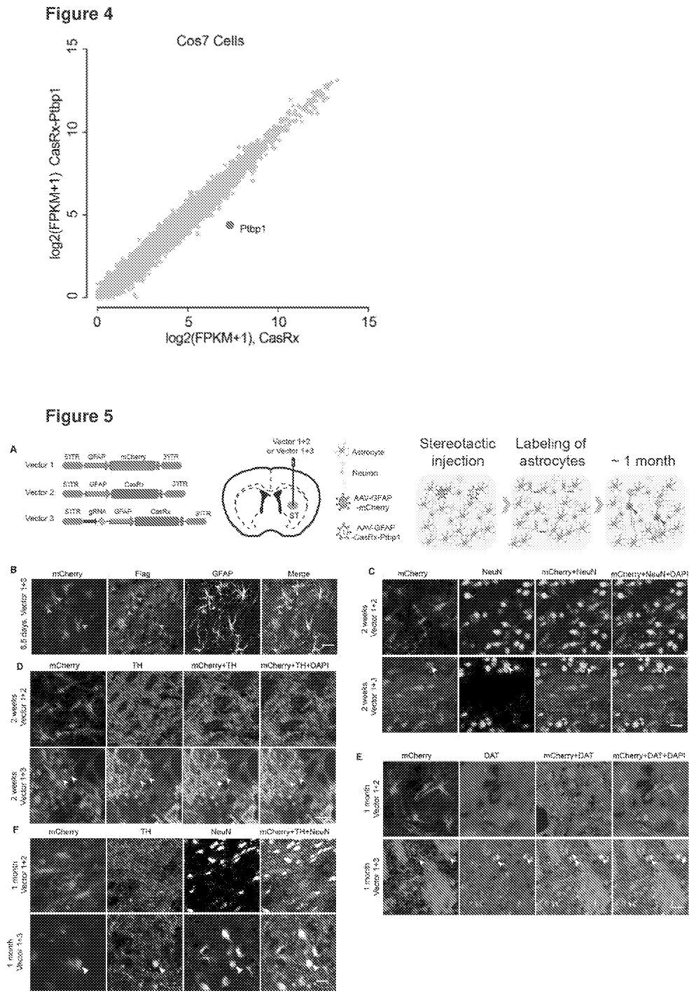

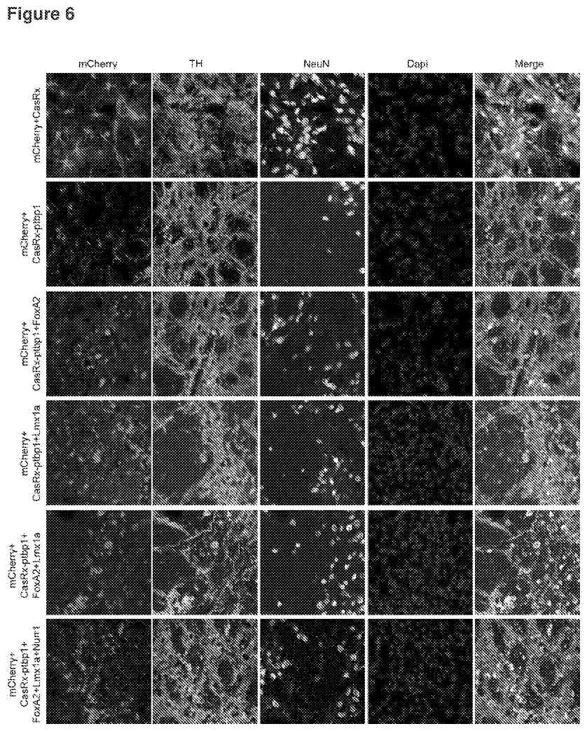

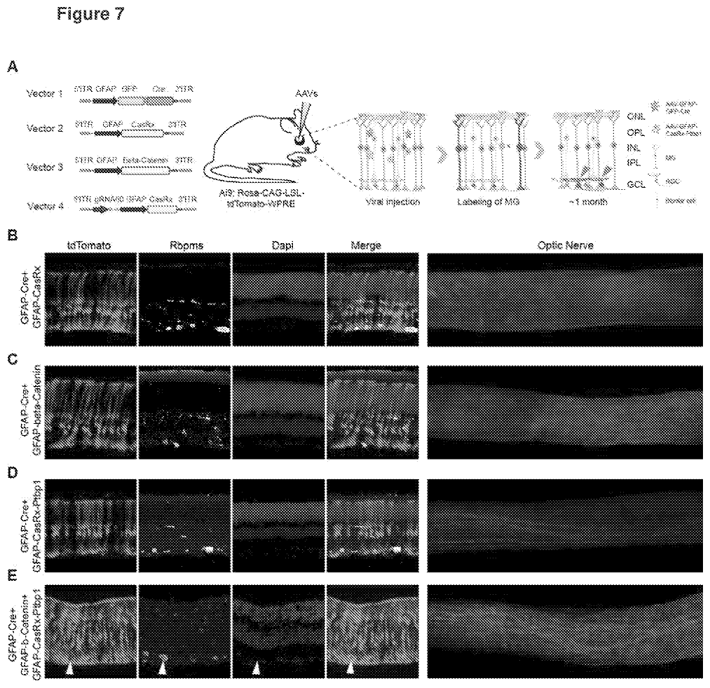

The targeting site of gRNA 60 is conserved in the Macaca fascicularis, human Ptbp1 and mouse gene and enabled the potent downregulation of Ptbp1 gene in human 293T, Monkey Cos7 cells and mouse N2a cells, thus it was used in the following experiments (FIGS. 2 and 3). To determine the targeting specificity of this strategy, we performed RNA-seq and found that Ptbp1 was specifically downregulated in Cos7 cells (FIG. 4). A recent study showed that Ptbp1 knockdown could convert striatal astrocytes into dopamine neurons in mice, we next examined whether Ptbp1 knockdown in the nonhuman primate striatal astrocytes could locally convert astrocytes into dopamine neurons in vivo. To validate the AAV vectors in vivo, we first injected wild type mice with AAV-GFAP-CasRx-Ptbp1 expressing CasRx and gRNA 60, together with AAV-GFAP-mCherry that fluorescently labeled astrocytes. We also constructed the control vector AAV-GFAP-CasRx that does not express Ptbp1 gRNA (FIG. 5A). One weeks after injection, we found that both mCherry and CasRx were specifically expressed in astrocytes and showed a high co-localization efficiency in the striatum. In addition, a high percentage of mCherry+ cells expressed mature neuron markers NeuN but not in the control striatum injected with AAV-GFAP-mCherry and AAV-GFAP-CasRx (FIGS. 5B and 5C). Moreover, a large fraction of mCherry+ cells expressed dopamine neuron markers TH at two weeks after AAV injection and DAT at one month after AAV injection (FIGS. 5D and 5E). To explore whether knockdown of Ptbp1 in Macaca fascicularis striatum could converted astrocytes into dopamine neuron. We injected AAV-GFAP-CasRx-Ptbp/together with AAV-GFAP-mCherry into the putamen of the right hemisphere of a Macaca fascicularis aged 8 years, and AAV-GFAP-CasRx together with AAV-GFAP-mCherry into the left putamen. One month after AAV injection, we observed mCherry⁺TH⁺ cells in the right putamen but not in the control putamen (FIG. 5F), demonstrating that knockdown of Ptbp1 could also convert astrocytes into dopamine neurons in nonhuman primates.

Moreover, we also explored the possibility of increasing the dopamine neuron conversion efficiency by combining other dopamine neuron conversion-related transcription factors, such as FoxA2, Lmx1a and Nurr1. We hypothesize that overexpression of these transcription factors may enhance dopmamine neuron conversion. Indeed, our results showed that co-expression of AAV-GFAP-CasRx-Ptbp1 with AAV-GFAP-FoxA2, AAV-GFAP-Lmx1a, AAV-GFAP-FoxA2+AAV-GFAP-Lmx1a or AAV-GFAP-FoxA2+AAV-GFAP-Lmx1a+AAV-GFAP-Nurr1 increased the efficiency of dopamine neuron conversion in mouse striatum, compared with expression of AAV-GFAP-CasRx-Ptbp/on its own (see FIG. 6).

Besides astrocyte-to-dopamine neuron conversion, our previous results showed that knockdown of Ptbp1 could convert Muller Glia (MG) into retinal ganglion cells (RGCs) in the mature retinas aged 4-8 weeks. However, it is unknown whether this strategy is applicable to middle-aged and old mice. We thus introduced AAV-GFAP-CasRx-Ptbp1 and AAV-GFAP-GFP-Cre into the eyes of ai9 mice (CAG-LSL-tdTomato) aged 4-5 months via subretina injection, and observed a small number of tdTomato⁺ axons in the optic nerve, 3 weeks after injection. To explore whether stimulation of MG re-cycling could increase the MG-to-RGC conversion, we co-injected AAV-GFAP-CasRx-Ptbp1, AAV-GFAP-GFP-Cre with AAV-GFAP-β-catenin into the retinas aged 4-5 months. Around 3 weeks after injection, we found that injection AAV-GFAP-CasRx-Ptbp1+AAV-GFAP-β-catenin+AAV-GFAP-GFP-Cre enhanced the number of converted RGCs compared to that of AAV-GFAP-CasRx-Ptbp1 and AAV-GFAP-GFP-Cre (see FIG. 7). Taken together, we provide an new strategy to enhance the RGC conversion in the middle-aged or old mammalian.

### Example 2

### Alleviation of Symptoms in PD Model Monkeys

To determine whether astrocyte-to-dopamine neuron conversion could be achieved in the putamen of monkey PD model, we perform histology to observe a large fraction mCherry+ cells express the dopamine neuron marker TH in the putamen injected with AAV-GFAP-mCherry and AAV-GFAP-CasRx-Ptbp1, but not in the putamen injected with control AAVs (AAV-GFAP-mCherry and AAV-GFAP-CasRx). We further reveal that a high percentage of mCherry cells also express nigra A9-type dopaminergic neuron markers dopamine transporter (DAT). Together, these results indicate successful induction of dopamine neurons via downregulating Ptbp1 expression in putamen astrocytes. Next, we evaluate whether induced dopamine neurons could alleviate the symptoms of Parkinson's disease, we used a video-based analysis of monkey movements. Our results show that the PD monkeys injected with AAV-GFAP-mCherry+AAV-GFAP-CasRx-Ptbp1 show significant alleviation of PD symptoms (an alleviation of around 15%) around one month after AAV injection. However, we do not observe obvious alleviation of PD symptoms in the control monkey injected with AAV-GFAP-mCherry+AAV-GFAP-CasRx that does encode gRNA targeting Ptbp1. To determine whether the induced dopamine neurons functioned as dopaminergic neurons in vivo, we perform 18F-Dopa PET, 18F-DTBZ PET and 18F-FP-CIT PET to detect presynaptic dopaminergic function, monitor the activity of vesicular monoamine transporter type 2 (VMAT2) and precisely quantify DAT density, respectively. We find the enhancement of these signals in the putamen injected with AAV-GFAP-mCherry and AAV-GFAP-CasRx-Ptbp1, but not for control AAVs. These results show the functionality of induced dopamine neurons in the intact monkey putamen.

### MG-to-RGC Conversion in the NMDA-Induced Retinal Injury Monkey Model (RGC)

To examine whether induced RGCs could replenish RGCs in retina injury monkey model, we intravitreally inject adult monkeys with N-methyl-D-aspartate (NMDA), which causes a loss of the majority of RGCs. Two-to-three weeks after NMDA injection, the eyes are either injected with AAV-GFAP-tdTomato+AAV-GFAP-CasRx-Ptbp1+AAV-GFAP-β-catenin, AAV-GFAP-tdTomato+AAV-GFAP-CasRx-Ptbp1, or control AAVs that do not encode gRNA targeting Ptbp1 (AAV-GFAP-tdTomato+AAV-GFAP-CasRx+AAV-GFAP-β-catenin, and AAV-GFAP-tdTomato+AAV-GFAP-CasRx). We further hypothesize that co-expression of OSK (Oct4 (also known as Pou5f1), Sox2 and Klf4 genes) with CasRx-Ptbp1 in MG in middle-aged or aged retinas may promote RGC conversion. Thus, we also inject AAV-GFAP-tdTomato+AAV-GFAP-CasRx-Ptbp1+AAV-GFAP-OSK into the retina. One to two months after AAV injection, we observe that the number of tdTomato⁺Rbpms⁺ cells in the GCL is increased in the retinas injected with AAV-GFAP-tdTomato+AAV-GFAP-CasRx-Ptbp1+AAV-GFAP-β-catenin, AAV-GFAP-tdTomato+AAV-GFAP-CasRx-Ptbp1+AAV-GFAP-OSK, or AV-GFAP-tdTomato+AAV-GFAP-CasRx-Ptbp1, but almost no such cells in the retinas injected with control AAVs, indicating successful induction of MG-to-RGC conversion in the NMDA-induced retinal injury monkey model. In addition, the number of tdTomato⁺Rbpms⁺ cells in the GCL is more frequently observed in the retinas injected with AAV-GFAP-tdTomato+AAV-GFAP-CasRx-Ptbp1+AAV-GFAP-β-catenin or AAV-GFAP-tdTomato+AAV-GFAP-CasRx-Ptbp1+AAV-GFAP-OSK, compared to retinas injected with AAV-GFAP-tdTomato+AAV-GFAP-CasRx-Ptbp1. In the visual system, RGCs sends their axons to the brain via optic nerve and formed central projections with the dorsal lateral geniculate nucleus (dLGN) and superior colliculus (SC) in the monkey brain. To explore whether MG-derived RGCs integrate into the visual system, we section the monkey brain and observe more tdTomato⁺ axons in the treated optic nerve injected with AAV-GFAP-tdTomato+AAV-GFAP-CasRx-Ptbp1+AAV-GFAP-β-catenin or AAV-GFAP-tdTomato+AAV-GFAP-CasRx-Ptbp1+AAV-GFAP-OSK than that of AAV-GFAP-tdTomato+AAV-GFAP-CasRx-Ptbp1, and almost no such axons in the control group. Notably, we detect tdTomato⁺ axons in the dLGN and SC, and tdTomato⁺ axons are more abundant in the contralateral part of the brain than the ipsilateral part, suggesting that induced RGCs form the right connections with the brain. To determine whether MG could be converted into retinal photoreceptors, we perform immunostaining and observe that a fraction of tdTomato⁺ cells expressed rod cell markers rhodopsin and GNAT1, and cone cell markers S-opsin, M-opsin and mCAR, in the retinas injected with AV-GFAP-tdTomato+AAV-GFAP-CasRx-Ptbp1+AAV-GFAP-β-catenin, AAV-GFAP-tdTomato+AAV-GFAP-CasRx-Ptbp1+AAV-GFAP-OSK, and AAV-GFAP-tdTomato+AAV-GFAP-CasRx-Ptbp1, indicating that cone and rod cells could also be induced from MG.

### Methods

### PD Model Monkeys

Adult (7-10-years-old) male Macaca fascicularis are used for this study. To generate non-human primate Parkinson's disease model, the animals are intravenously injected with MPTP (1-methyl 4-phenyl 1,2,3,6-tetrahydropyridine) to deplete dopamine neurons in the substantia nigra until we observed persistent symptoms, such as tremor, bradykinesia, and impaired balance. Stable symptoms are observed for more than 5 weeks before AAV injection.

### Positron Emission Tomographic (PET) Study (PD Part)

To monitor the dopaminergic function of converted dopamine neurons in the intact monkey brains, PET imaging (18F-DOPA, 18F-DTBZ and 18F-FP-CIT) is performed before and around one month after AAV injection. Before scanning, monkeys are anaesthetized and then the animals are placed in the PET scanner and all physiological parameters were monitored. The PET data are analyzed using standard protocol after scanning.

### Video Analysis of Behavioral Recovery (PD Part)

For video analysis, the movement of monkeys are recorded before and one, two months after AAV injection. The recorded videos are analyzed after collection. Monkey PD symptoms are evaluated using the following items: head checking movements, facial expression, spontaneous activity, movements in response to stimuli, tremor, posture and gait.

### Generation of NMDA-Induced Retinal Injury Monkey Model and Injection of AAV (RGC Part)

NMDA and AAVs are injected via intravitreal and subretinal injection, respectively. For intravitreal injection, then monkeys are anaesthetized and alcaine is droped on the eyes. The NMDA solution is injected into the vitreous body to deplete the majority of RGCs, and then ofloxacin eye ointment is introduced on the eye to prevent infection. For subretinal injection, monkeys are anaesthetized two-three weeks after NMDA injection and the pupil size is dilated. The AAVs is slowly injected into the subretinal space. The needle is removed and the eye ointment is administrated after injection. To determine the conversion, we designed three strategies to induce MG-to-RGC conversion: AAV-GFAP-tdTomato+AAV-GFAP-CasRx-Ptbp1+AAV-GFAP-β-catenin, AAV-GFAP-tdTomato+AAV-GFAP-CasRx-Ptbp1+AAV-GFAP-OSK, and AAV-GFAP-tdTomato+AAV-GFAP-CasRx-Ptbp1. In addition, we inject the control AAVs that does not encode gRNAs targeting Ptbp1: AAV-GFAP-tdTomato+AAV-GFAP-CasRx+AAV-GFAP-β-catenin, AAV-GFAP-tdTomato+AAV-GFAP-CasRx+AAV-GFAP-OSK, or AAV-GFAP-tdTomato+AAV-GFAP-CasRx. We note that transcription of gRNA is driven by the U6 promoter. AAVs are subretinally delivered into the retinas. Around one-to-two months after AAV injection, the eyes, optic nerves and brain are extracted and fixed with 4% paraformaldehyde (PFA), and then maintain in a 30% sucrose solution. After embedding, eyes and brains are sectioned, incubated with antibodies and visualized under a microscope.

1. A method of treating a neurological condition associated with degeneration of functional neurons in a region in the nervous system of a non-human primate or a human, the method comprising the administration to a non-neuronal cell in the region in the nervous system of a subject in need thereof, an effective amount of a composition comprising a nucleic acid molecule that targets a target sequence or its complement in a primate PTB gene, transcript or mRNA, to suppress the expression or activity of a primate PTB protein in the non-neuronal cell, thereby allowing the non-neural cell to reprogram into a functional neuron, wherein the target sequence is comprised in a sequence that is at least 95% identical to SEQ ID NO: 87, and wherein when the target sequence is targeted with a guide RNA (gRNA), the guide sequence of which has at least 10 nucleotides overlap with at least one of SEQ ID NO.'s: 1-86, that is co-expressed with CasRx in at least one of Cos7 and 293T cells, a relative expression of PTB mRNA of less than 0.5 is observed, as compared to a corresponding control cell expressing only CasRx.

2. A method of treating a neurological condition associated with degeneration of functional neurons in a region in the nervous system of a non-human primate or a human, the method comprising the administration to a non-neuronal cell in the region in the nervous system of a subject in need thereof, an effective amount of a composition comprising a nucleic acid molecule that targets a target sequence or its complement in a primate PTB gene, transcript or mRNA, to suppress the expression or activity of PTB in the non-neuronal cell, thereby allowing the non-neural cell to reprogram into a functional neuron, wherein the target sequence is comprised in a sequence that is at least 95% identical to positions 951-1487 of SEQ ID NO: 87.

3. The method of claim 2, wherein when the target sequence is targeted with a guide RNA (gRNA), the guide sequence of which has at least 10 nucleotides overlap with at least one of SEQ ID NO.'s: 38-68, that is co-expressed with CasRx in at least one of Cos7 and 293T cells, a relative expression of PTB mRNA of less than 0.4 is observed, as compared to a corresponding control cell expressing only CasRx.

4. The method of any one of the preceding claims, wherein the nucleic acid molecule is or encodes at least one of an antisense nucleic acid, an RNAi molecule or a guide RNA (gRNA).

5. The method of claim 4, wherein composition comprises: i) a Cas effector protein and at least one gRNA or ii) at least one expression vector encoding a Cas effector protein and encoding the at least one gRNA, wherein optionally, the Cas effector protein and the at least one gRNA or the at least one expression vector are comprised in a nanoparticle, preferably a liposome.

6. The method of claim 5, wherein:
a) the Cas effector protein is an RNA-targeting Cas effector protein; and,
b) the gRNA comprises a guide sequence that is complementary to a contiguous stretch of 17-60 nucleotides in a sequence that is at least 95% identical to SEQ ID NO: 87.

7. The method of claim 6, wherein the RNA-targeting Cas effector protein selected from the group consisting of: Cas13d, CasRx, Cas13e, Cas13a, Cas13b, Cas13c, Cas13f and a functional domain thereof, of which CasRx is preferred.

8. The method of claim 7, wherein the guide sequence in the gRNA comprises or consists of at least 10, 11, 12, 13, 14, 15, 16, 17 contiguous nucleotides, or of all nucleotides of at least one of SEQ ID NO.'s: 1-86, and wherein, preferably, when the gRNA is co-expressed with CasRx in at least one of Cos7 and 293T cells, a relative expression of PTB mRNA of less than 0.5, 0.45, 0.4, 0.35, 0.3, 0.25, 0.24, 0.23, 0.22, 0.21, 0.20, 0.19, 0.18, 0.17, 0.16, 0.15, 0.14, 0.13, 0.12, 0.11, 0.09, 0.08, 0.07, 0.06, 0.05, 0.04, 0.03, 0.02 or 0.015 is observed, as compared to a corresponding control cell expressing only CasRx.

9. The method of claim 8, wherein the guide sequence in the gRNA comprises or consists of at least one sequence selected from SEQ ID NO.'s: 56, 60, 47, 48, 43, 45, 40, 61, 50, 55, 41, 44, 42, 66, 49, 51 and 46.

10. The method of any one of the preceding claims, wherein the composition comprises only a single type of gRNA or two, three, four five or six different gRNAs that target a primate PTB mRNA sequence or the at least one expression vector encodes only a single type of gRNA or two, three, four five or six different gRNAs that target a primate PTB mRNA sequence.

11. The method of any one of the preceding claims, wherein the at least one expression vector comprises:
i) a nucleotide sequence encoding the Cas effector protein that is operably linked to a promoter that causes expression of the Cas effector protein in a primate non-neuronal cell, wherein preferably the promoter is a glial cell-specific promoter or a Müller glia (MG) cell-specific promoter, wherein more preferably a glial cell-specific promoter selected from the GFAP promoter, the ALDH1L1 promoter, the EAAT1/GLAST promoter, the glutamine synthetase promoter, S100 beta promoter and the EAAT2/GLT-1 promoter, or an MG cell-specific promoter selected from the GFAP promoter, the ALDH1L1 promoter, Glast (also known as Slc1a3) promoter and the Rlbp1 promoter; and,
ii) at least one nucleotide sequence encoding a gRNA that targets a primate PTB mRNA sequence, which nucleotide sequence is operably linked to a promoter that causes expression of the gRNA in the non-neuronal cell, such as a U6 promoter.

12. The method of any one of the preceding claims, wherein the expression vector is comprised in a nanoparticle or wherein the expression vector is a gene therapy vector, preferably a viral gene therapy vector, more preferably a viral vector selected from the group consisting of an adeno-associated virus (AAV) vector, an adenovirus vector, a lentivirus vector, a retrovirus vector, a herpes virus, a SV40 vector, a poxvirus vector, and a combination thereof, of which AAV is most preferred.

13. The method of any one of the preceding claims, wherein the composition is administered locally to at least one of: i) a non-neuronal cell in a mature retina; ii) a non-neuronal cell in the striatum, preferably putamen; iii) a non-neuronal cell in the substantia nigra, iv) a non-neuronal cell in an inner ear; v) a non-neuronal cell in the spinal cord; vi) a non-neuronal cell in the prefrontal cortex; vii) a non-neuronal cell in the motor cortex; and, viii) a non-neuronal cell in the ventral tegmental area (VTA).

14. The method of claim 13, wherein the composition is administered to a non-neuronal cell in the striatum for generating a functional dopaminergic neuron, whereby, preferably the non-neuronal cell is a glial cell, wherein preferably the composition is administered to at least one of the putamen and substantia nigra.

15. The method of claim 14, wherein the glial cell is an astrocyte.

16. The method of any one of claims 13-15, wherein, the neurological condition is a condition associated with degeneration of functional neurons is selected from the group consisting of: Parkinson's disease; Alzheimer's disease; Huntington's disease; Schizophrenia; depression; drug addiction; stroke; movement disorder such as chorea; spinal cord injury; choreoathetosis, and dyskinesias; bipolar disorder; Autism spectrum disorder (ASD); and dysfunction.

17. The method of any one of claims 13-15, wherein the composition further comprises i) one or more dopamine neuron-associated factors, or ii) at least one expression vector for expression of one or more dopamine neuron-associated factors in the non-neuronal cell.

18. The method of claim 17, wherein the one or more dopamine neuron-associated factors are selected from the group consisting of: Lmx1a, Lmx1b, FoxA2, Nurr1, Pitx3, Gata2, Gata3, FGF8, BMP, En1, En2, PET1, a Pax family protein, SHH, a Wnt family protein and a TGF-β family protein.

19. The method of claim 12, wherein the composition is administered to a glial cell or Müller glia (MG) cell in the mature retina for generating a functional retinal ganglion cell (RGC) neuron and/or a functional retinal photoreceptor.

20. The method of claim 12 or 19, wherein the neurological condition is a condition associated with degeneration of functional neurons in the mature retina is selected from the group consisting of: glaucoma, age-related RGC loss, optic nerve injury, retinal ischemia and Leber's hereditary optic neuropathy.

21. The method of claim 19 or 20, wherein the composition further comprises i) one or more factors selected from β-catenin, Oct4, Sox2, Klf4, Crx, Brn3a, Brn3b, Math5, Nr2e3 and Nrl, and/or ii) at least one expression vector for expression in a non-neuronal cell of one or more factors selected from β-catenin, Oct4, Sox2, Klf4, Crx, Brn3a, Brn3b, Math5, Nr2e3 and Nrl.

22. The method according to any one of the preceding claims, wherein the method further comprises administering at least one immunosuppressant, prior to, simultaneously with or after the administration of the cell-programming agent, wherein more preferably, the at least one immunosuppressant is selected from the group consisting of: a corticosteroid, a calcineurin inhibitor, an mTOR inhibitor, an IMDH inhibitor, an immuno suppressive antibody, an interferon, a Janus kinase inhibitor and a biologic such as anakinra.

23. A composition comprising a nucleic acid molecule that targets a target sequence or its complement in a primate PTB gene, transcript or mRNA, as defined in any one of claims 1-4.

24. The composition of claim 23, wherein composition comprises: i) a Cas effector protein and at least one gRNA or ii) at least one expression vector encoding a Cas effector protein and encoding the at least one gRNA, wherein optionally, the Cas effector protein and the at least one gRNA or the at least one expression vector are comprised in a nanoparticle, preferably a liposome.

25. The composition of claim 24, wherein the Cas effector protein and the at least one gRNA are as defined in any one of claims 6-10.

26. The composition of claim 24 or 25, wherein the expression vector is an expression vector as defined in claim 11 or 12.

27. The composition of any one of claims 23-26, wherein the composition further comprises i) one or more dopamine neuron-associated factors, or ii) at least one expression vector for expression of one or more dopamine neuron-associated factors in the non-neuronal cell, wherein preferably, the one or more dopamine neuron-associated factors are selected from the group consisting of: Lmx1a, Lmx1b, FoxA2, Nurr1, Pitx3, Gata2, Gata3, FGF8, BMP, En1, En2, PET1, a Pax family protein, SHH, a Wnt family protein and a TGF-β family protein.

28. The composition of any one of claims 23-26, wherein the composition further comprises i) one or more factors selected from β-catenin, Oct4, Sox2, Klf4, Crx, Brn3a, Brn3b, Math5, Nr2e3 and Nrl, and/or ii) at least one expression vector for expression in a non-neuronal cell of one or more factors selected from β-catenin, Oct4, Sox2, Klf4, Crx, Brn3a, Brn3b, Math5, Nr2e3 and Nrl.

29. The composition of any one of claims 23-28, formulated for injection, inhalation, parenteral administration, intravenous administration, subcutaneous administration, intramuscular administration, intradermal administration, topical administration, or oral administration.

30. An AAV vector, comprising:
(a) a coding sequence of an RNA-targeting Cas effector protein; and
(b) at least one nucleotide sequence encoding a gRNA as defined in any one of claims 6-10.

31. The AAV vector of claim 30, wherein the RNA-targeting Cas effector protein selected from the group consisting of: Cas13d, CasRx, Cas13e, Cas13a, Cas13b, Cas13c, Cas13f and a functional domain thereof, of which CasRx is preferred.

32. The AAV vector of claim 31, wherein:
i) the nucleotide sequence encoding the Cas effector protein that is operably linked to a promoter that causes expression of the Cas effector protein in a primate non-neuronal cell, wherein preferably the promoter is a glial cell-specific promoter or a Müller glia (MG) cell-specific promoter, wherein more preferably a glial cell-specific promoter selected from the GFAP promoter, the ALDH1L1 promoter, the EAAT1/GLAST promoter, the glutamine synthetase promoter, S100 beta promoter and the EAAT2/GLT-1 promoter, or an MG cell-specific promoter selected from the GFAP promoter, the ALDH1L1 promoter, Glast (also known as Slc1a3) promoter and the Rlbp1 promoter; and,
ii) the at least one nucleotide sequence encoding a gRNA is operably linked to a promoter that causes expression of the gRNA in the non-neuronal cell, such as a U6 promoter.


SEQUENCE LISTING (88 sequences)

SEQ ID NO: 1 - RNA, 31 nt, Artificial Sequence
  Ptbp1-gRNA-#
uccccgcuuuguaccaacggcuauaucuggg

SEQ ID NO: 2 - RNA, 30 nt, Artificial Sequence
  Ptbp1-gRNA-#
uaaacgguccguuagugacacaaguagaga

SEQ ID NO: 3 - RNA, 30 nt, Artificial Sequence
  Ptbp1-gRNA-#
ugauaaacgguccguuagugacacaaguag

SEQ ID NO: 4 - RNA, 25 nt, Artificial Sequence
  Ptbp1-gRNA-#
cgguccguuagugacacaaguagag

SEQ ID NO: 5 - RNA, 25 nt, Artificial Sequence
  Ptbp1-gRNA-#
cagaagccgaguugcugcucaugau

SEQ ID NO: 6 - RNA, 29 nt, Artificial Sequence
  Ptbp1-gRNA-#
cuucuugcugucauuuccguuugcugcag

SEQ ID NO: 7 - RNA, 27 nt, Artificial Sequence
  Ptbp1-gRNA-#
acgccugcacuucggcugucaccuuug

SEQ ID NO: 8 - RNA, 25 nt, Artificial Sequence
  Ptbp1-gRNA-#
agcuuccggauguggaucacucuag

SEQ ID NO: 9 - RNA, 32 nt, Artificial Sequence
  Ptbp1-gRNA-#
agaugacuucccccuccgugacgucgaugggg

SEQ ID NO: 10 - RNA, 30 nt, Artificial Sequence
  Ptbp1-gRNA-#
cuucagcaucaggagguuggugaccuuccc

SEQ ID NO: 11 - RNA, 30 nt, Artificial Sequence
  Ptbp1-gRNA-#
gccuccuccguguucaucucgaugaaggcc

SEQ ID NO: 12 - RNA, 30 nt, Artificial Sequence
  Ptbp1-gRNA-#
uuggcagccuccuccguguucaucucgaug

SEQ ID NO: 13 - RNA, 29 nt, Artificial Sequence
  Ptbp1-gRNA-#
ccaugguguuggcagccuccuccguguuc

SEQ ID NO: 14 - RNA, 29 nt, Artificial Sequence
  Ptbp1-gRNA-#
guguaguaguucaccaugguguuggcagc

SEQ ID NO: 15 - RNA, 31 nt, Artificial Sequence
  Ptbp1-gRNA-#
ccgagguguaguaguucaccaugguguuggc

SEQ ID NO: 16 - RNA, 30 nt, Artificial Sequence
  Ptbp1-gRNA-#
cuggauguagaugggcuggccgcgcagcac

SEQ ID NO: 17 - RNA, 30 nt, Artificial Sequence
  Ptbp1-gRNA-#
gcuccuugugguuggagaacuggauguaga

SEQ ID NO: 18 - RNA, 30 nt, Artificial Sequence
  Ptbp1-gRNA-#
cugucggucuucagcuccuugugguuggag

SEQ ID NO: 19 - RNA, 31 nt, Artificial Sequence
  Ptbp1-gRNA-#
gagcugucggucuucagcuccuugugguugg

SEQ ID NO: 20 - RNA, 30 nt, Artificial Sequence
  Ptbp1-gRNA-#
gggccgccugggcccgcgccugguugggag

SEQ ID NO: 21 - RNA, 30 nt, Artificial Sequence
  Ptbp1-gRNA-#
ggccagguuccccgacuggaccgaguucac

SEQ ID NO: 22 - RNA, 26 nt, Artificial Sequence
  Ptbp1-gRNA-#
cguccacggccgccgccgaggcagcc

SEQ ID NO: 23 - RNA, 30 nt, Artificial Sequence
  Ptbp1-gRNA-#
gguucuccacgaugauccugagcacggggc

SEQ ID NO: 24 - RNA, 30 nt, Artificial Sequence
  Ptbp1-gRNA-#
gaagagguucuccacgaugauccugagcac

SEQ ID NO: 25 - RNA, 27 nt, Artificial Sequence
  Ptbp1-gRNA-#
ugcagcacauccagggucacaggguag

SEQ ID NO: 26 - RNA, 30 nt, Artificial Sequence
  Ptbp1-gRNA-#
augaucuucaacacugugccgaacuuggag

SEQ ID NO: 27 - RNA, 30 nt, Artificial Sequence
  Ptbp1-gRNA-#
cugguuguucuuggugaaggugaugaucuu

SEQ ID NO: 28 - RNA, 30 nt, Artificial Sequence
  Ptbp1-gRNA-#
gcagggccuggaacugguuguucuugguga

SEQ ID NO: 29 - RNA, 30 nt, Artificial Sequence
  Ptbp1-gRNA-#
cugcagcagggccuggaacugguuguucuu

SEQ ID NO: 30 - RNA, 25 nt, Artificial Sequence
  Ptbp1-gRNA-#
guccgcauacugcagcagggccugg

SEQ ID NO: 31 - RNA, 30 nt, Artificial Sequence
  Ptbp1-gRNA-#
uguucugcccguccagcgacagcuuggcgu

SEQ ID NO: 32 - RNA, 36 nt, Artificial Sequence
  Ptbp1-gRNA-#
cagcaggcguuguagauguucugcccguccagcgac

SEQ ID NO: 33 - RNA, 30 nt, Artificial Sequence
  Ptbp1-gRNA-#
gcgugcagcaggcguuguagauguucugcc

SEQ ID NO: 34 - RNA, 30 nt, Artificial Sequence
  Ptbp1-gRNA-#
agucgaugcgcagcgugcagcaggcguugu

SEQ ID NO: 35 - RNA, 31 nt, Artificial Sequence
  Ptbp1-gRNA-#
uuguacuugacguugaggcuggugagcuugg

SEQ ID NO: 36 - RNA, 30 nt, Artificial Sequence
  Ptbp1-gRNA-#
uuguuguacuugacguugaggcuggugagc

SEQ ID NO: 37 - RNA, 30 nt, Artificial Sequence
  Ptbp1-gRNA-#
ucugguccagcgagggcuggcuguccccgg

SEQ ID NO: 38 - RNA, 28 nt, Artificial Sequence
  Ptbp1-gRNA-#
agaggcugagauuauaccaggugcaccg

SEQ ID NO: 39 - RNA, 33 nt, Artificial Sequence
  Ptbp1-gRNA-#
ccugcagcuugaggaauggcaaaggugggaggg

SEQ ID NO: 40 - RNA, 27 nt, Artificial Sequence
  Ptbp1-gRNA-#
ccagggcgccguggacguucggaacgg

SEQ ID NO: 41 - RNA, 25 nt, Artificial Sequence
  Ptbp1-gRNA-#
ggauggcgauccgaccugccgccgc

SEQ ID NO: 42 - RNA, 30 nt, Artificial Sequence
  Ptbp1-gRNA-#
gguugcugaccagcaauacagaauuuccug

SEQ ID NO: 43 - RNA, 37 nt, Artificial Sequence
  Ptbp1-gRNA-#
acucucucuggguugagguugcugaccagcaauacag

SEQ ID NO: 44 - RNA, 30 nt, Artificial Sequence
  Ptbp1-gRNA-#
cucucucuggguugagguugcugaccagca

SEQ ID NO: 45 - RNA, 30 nt, Artificial Sequence
  Ptbp1-gRNA-#
acucucucuggguugagguugcugaccagc

SEQ ID NO: 46 - RNA, 34 nt, Artificial Sequence
  Ptbp1-gRNA-#
ugugacucucucuggguugagguugcugaccagc

SEQ ID NO: 47 - RNA, 30 nt, Artificial Sequence
  Ptbp1-gRNA-#
aggcuuuggggugugacucucucuggguug

SEQ ID NO: 48 - RNA, 30 nt, Artificial Sequence
  Ptbp1-gRNA-#
cugcacgucaccguagacgccgaaaagaau

SEQ ID NO: 49 - RNA, 33 nt, Artificial Sequence
  Ptbp1-gRNA-#
aucuucacgcgcugcacgucaccguagacgccg

SEQ ID NO: 50 - RNA, 39 nt, Artificial Sequence
  Ptbp1-gRNA-#
ccguccgccaucugcacuagggcguucuccuucuuauug

SEQ ID NO: 51 - RNA, 30 nt, Artificial Sequence
  Ptbp1-gRNA-#
gcugggccugguugccguccgccaucugca

SEQ ID NO: 52 - RNA, 40 nt, Artificial Sequence
  Ptbp1-gRNA-#
gcucauggccagcugggccugguugccguccgccaucugc

SEQ ID NO: 53 - RNA, 30 nt, Artificial Sequence
  Ptbp1-gRNA-#
uggccagcugggccugguugccguccgcca

SEQ ID NO: 54 - RNA, 36 nt, Artificial Sequence
  Ptbp1-gRNA-#
uggcucauggccagcugggccugguugccguccgcc

SEQ ID NO: 55 - RNA, 34 nt, Artificial Sequence
  Ptbp1-gRNA-#
cuucccgugcagcuugugcccguucagguggcuc

SEQ ID NO: 56 - RNA, 25 nt, Artificial Sequence
  Ptbp1-gRNA-#
cagcugcacguucuggugcuucgag

SEQ ID NO: 57 - RNA, 30 nt, Artificial Sequence
  Ptbp1-gRNA-#
guugccguaguccuuggucaggcccugguc

SEQ ID NO: 58 - RNA, 30 nt, Artificial Sequence
  Ptbp1-gRNA-#
gcccggcuucuugaagcggugcagggguga

SEQ ID NO: 59 - RNA, 30 nt, Artificial Sequence
  Ptbp1-gRNA-#
guucuuggagcccggcuucuugaagcggug

SEQ ID NO: 60 - RNA, 30 nt, Artificial Sequence
  Ptbp1-gRNA-#
uucuggaaguucuuggagcccggcuucuug

SEQ ID NO: 61 - RNA, 36 nt, Artificial Sequence
  Ptbp1-gRNA-#
aauauguucuggaaguucuuggagcccggcuucuug

SEQ ID NO: 62 - RNA, 30 nt, Artificial Sequence
  Ptbp1-gRNA-#
uauguucuggaaguucuuggagcccggcuu

SEQ ID NO: 63 - RNA, 30 nt, Artificial Sequence
  Ptbp1-gRNA-#
gggcgggaauauguucuggaaguucuugga

SEQ ID NO: 64 - RNA, 28 nt, Artificial Sequence
  Ptbp1-gRNA-#
cguggccgagggcgggaauauguucugg

SEQ ID NO: 65 - RNA, 33 nt, Artificial Sequence
  Ptbp1-gRNA-#
auguuggagaggugcagcguggccgagggcggg

SEQ ID NO: 66 - RNA, 27 nt, Artificial Sequence
  Ptbp1-gRNA-#
agauccuccucggagacugagggcggg

SEQ ID NO: 67 - RNA, 32 nt, Artificial Sequence
  Ptbp1-gRNA-#
cuugaauccuuugacgacgcccccauugcugg

SEQ ID NO: 68 - RNA, 30 nt, Artificial Sequence
  Ptbp1-gRNA-#
cuugaauccuuugacgacgcccccauugcu

SEQ ID NO: 69 - RNA, 29 nt, Artificial Sequence
  Ptbp1-gRNA-#
ccaucuugcgguccuucuggaagaacuug

SEQ ID NO: 70 - RNA, 30 nt, Artificial Sequence
  Ptbp1-gRNA-#
gugccaucuugcgguccuucuggaagaacu

SEQ ID NO: 71 - RNA, 36 nt, Artificial Sequence
  Ptbp1-gRNA-#
cccaucuggaucagugccaucuugcgguccuucugg

SEQ ID NO: 72 - RNA, 30 nt, Artificial Sequence
  Ptbp1-gRNA-#
aucagugccaucuugcgguccuucuggaag

SEQ ID NO: 73 - RNA, 27 nt, Artificial Sequence
  Ptbp1-gRNA-#
aucagugccaucuugcgguccuucugg

SEQ ID NO: 74 - RNA, 30 nt, Artificial Sequence
  Ptbp1-gRNA-#
gcccaucuggaucagugccaucuugcgguc

SEQ ID NO: 75 - RNA, 32 nt, Artificial Sequence
  Ptbp1-gRNA-#
ccuggaccgccuccuccacggagcccaucugg

SEQ ID NO: 76 - RNA, 29 nt, Artificial Sequence
  Ptbp1-gRNA-#
uucuccccgaggucgugguugugcagguc

SEQ ID NO: 77 - RNA, 30 nt, Artificial Sequence
  Ptbp1-gRNA-#
gaaggagacccgcagguggugguucucccc

SEQ ID NO: 78 - RNA, 30 nt, Artificial Sequence
  Ptbp1-gRNA-#
ugguggacuuggagaaggagacccgcaggu

SEQ ID NO: 79 - RNA, 28 nt, Artificial Sequence
  Ptbp1-gRNA-#
ccugugccccuagaugguggacuuggag

SEQ ID NO: 80 - RNA, 28 nt, Artificial Sequence
  Ptbp1-gRNA-#
ucccugucaccgcagggugagcaaggug

SEQ ID NO: 81 - RNA, 27 nt, Artificial Sequence
  Ptbp1-gRNA-#
ccgggaacucccgcugccacagucacc

SEQ ID NO: 82 - RNA, 28 nt, Artificial Sequence
  Ptbp1-gRNA-#
acaccccggguggguguguggcugcagg

SEQ ID NO: 83 - RNA, 29 nt, Artificial Sequence
  Ptbp1-gRNA-#
uauugcuaggcacagacgucugcaacugc

SEQ ID NO: 84 - RNA, 26 nt, Artificial Sequence
  Ptbp1-gRNA-#
cuggguccugccugugacuuaauuac

SEQ ID NO: 85 - RNA, 30 nt, Artificial Sequence
  Ptbp1-gRNA-#
acccacugggcggccccgccugcucucugg

SEQ ID NO: 86 - RNA, 28 nt, Artificial Sequence
  Ptbp1-gRNA-#
cccccgggacaggacgcgggggcgguag

SEQ ID NO: 87 - DNA, 1674 bp, Homo sapiens
atggacggcattgtcccagatatagccgttggtacaaagcggggatctgacgagcttttctctacttgtgtcactaacggaccgtttatcatgagcagcaactcggcttctgcagcaaacggaaatgacagcaagaagttcaaaggtgacagccgaagtgcaggcgtcccctctagagtgatccacatccggaagctccccatcgacgtcacggagggggaagtcatctccctggggctgccctttgggaaggtcaccaacctcctgatgctgaaggggaaaaaccaggccttcatcgagatgaacacggaggaggctgccaacaccatggtgaactactacacctcggtgacccctgtgctgcgcggccagcccatctacatccagttctccaaccacaaggagctgaagaccgacagctctcccaaccaggcgcgggcccaggcggccctgcaggcggtgaactcggtccagtcggggaacctggccttggctgcctcggcggcggccgtggacgcagggatggcgatggccgggcagagccccgtgctcaggatcatcgtggagaacctcttctaccctgtgaccctggatgtgctgcaccagattttctccaagttcggcacagtgttgaagatcatcaccttcaccaagaacaaccagttccaggccctgctgcagtatgcggaccccgtgagcgcccagcacgccaagctgtcgctggacgggcagaacatctacaacgcctgctgcacgctgcgcatcgacttttccaagctcaccagcctcaacgtcaagtacaacaatgacaagagccgtgactacacacgcccagacctgccttccggggacagccagccctcgctggaccagaccatggccgcggccttcggtgcacctggtataatctcagcctctccgtatgcaggagctggtttccctcccacctttgccattcctcaagctgcaggcctttccgttccgaacgtccacggcgccctggcccccctggccatcccctcggcggcggcggcagctgcggcggcaggtcggatcgccatcccgggcctggcgggggcaggaaattctgtattgctggtcagcaacctcaacccagagagagtcacaccccaaagcctctttattcttttcggcgtctacggtgacgtgcagcgcgtgaagatcctgttcaataagaaggagaacgccctagtgcagatggcggacggcaaccaggcccagctggccatgagccacctgaacgggcacaagctgcacgggaagcccatccgcatcacgctctcgaagcaccagaacgtgcagctgccccgcgagggccaggaggaccagggcctgaccaaggactacggcaactcacccctgcaccgcttcaagaagccgggctccaagaacttccagaacatattcccgccctcggccacgctgcacctctccaacatcccgccctcagtctccgaggaggatctcaaggtcctgttttccagcaatgggggcgtcgtcaaaggattcaagttcttccagaaggaccgcaagatggcactgatccagatgggctccgtggaggaggcggtccaggccctcattgacctgcacaaccacgacctcggggagaaccaccacctgcgggtctccttctccaagtccaccatctag

SEQ ID NO: 88 - DNA, 536 bp, Homo sapiens
gccattcctcaagctgcaggcctttccgttccgaacgtccacggcgccctggcccccctggccatcccctcggcggcggcggcagctgcggcggcaggtcggatcgccatcccgggcctggcgggggcaggaaattctgtattgctggtcagcaacctcaacccagagagagtcacaccccaaagcctctttattcttttcggcgtctacggtgacgtgcagcgcgtgaagatcctgttcaataagaaggagaacgccctagtgcagatggcggacggcaaccaggcccagctggccatgagccacctgaacgggcacaagctgcacgggaagcccatccgcatcacgctctcgaagcaccagaacgtgcagctgccccgcgagggccaggaggaccagggcctgaccaaggactacggcaactcacccctgcaccgcttcaagaagccgggctccaagaacttccagaacatattcccgccctcggccacgctgcacctctccaacatcccgccctcagtctccgaggaggatct